(19) United States
(12) Patent Application Publication
(10) Pub. No.: US 2006/0239990 A1
(43) Pub. Date: Oct. 26, 2006

(54) Protein Arginine N-Methyltransferase 2 (PRMT-2)

(76) Inventors: Elizabeth G. Nabel, Washington, DC (US); Gary J. Nabel, Washington, DC (US); Takanobu Yoshimoto, Tokyo (JP); Hiroaki Iwasaki, North Bethesda, MD (US)

Correspondence Address: Schwegman, Lundberg, Woessner & Kluth, P.A., P.O. Box 2938, Minneapolis, MN, 55402, US

(21) Appl. No.: 11/263,657

(22) Filed: Oct. 31, 2005

Related U.S. Application Data
(63) Continuation-in-part of application No. PCT/US04/13375, filed on Apr. 30, 2004.
(60) Provisional application No. 60/466,751, filed on Apr. 30, 2003.

(51) Int. Cl. A61K 48/00; A61K 38/48

(52) U.S. Cl. 424/94.5; 514/44

(57) ABSTRACT

The invention provides insight into the function of Protein Arginine N-Methyltransferase-2 (PRMT-2) and provides methods for modulating PRMT-2 activity or expression in cells. The methods of the invention can be used to inhibit the function of NFκB, E2F1 and STAT3 and have utility for treating a variety of conditions including, for example, inflammation, HIV infection, cancer and obesity.

This application is a continuation-in-part of PCT Application Ser. No. PCT/US2004/013375 filed Apr. 30, 2004 and published on Nov. 18, 2004 as WO 2004/098634, which claims benefit of U.S. Provisional Application Ser. No. 60/466,751 filed Apr. 30, 2003, which applications and publication are incorporated herein by reference.

### GOVERNMENT FUNDING

The invention described herein was developed with support from the National Institutes of Health. The U.S. Government has certain rights in the invention.

### FIELD OF THE INVENTION

The present invention relates to Protein Arginine N-Methyltransferase 2 (PRMT-2) proteins and nucleic acids that have a variety of biological effects on mammals. For example, according to the invention PRMT-2 proteins and nucleic acids can modulate the activity of nuclear factor kappa B (NFκB) and therefore PRMT-2 has a role in modulating inflammation and the immune response. Also, as illustrated herein, PRMT-2 proteins can repress E2F1 transcriptional activity, arrest the cell cycle and thus may be used to treat or prevent cancer. Moreover, as described herein, PRMT-2 methylates STAT3 and inhibition or loss of PRMT-2 function causes mammals to loose weight, eat less and become more sensitive to insulin.

### BACKGROUND OF THE INVENTION

Protein-arginine methyltransferases catalyze the post-translational methylation of arginine residues in proteins, resulting in the mono- and di-methylation of arginine on the guanidino group. Known substrates are histones, heterogeneous nuclear ribonucleoproteins (hnRNPs), and myelin basic protein. Such post-translational modification is common in hnRNPs and may regulate their function.

The PRMT family consists of at least five members, including PRMT-1, PRMT-2, PRMT-3, CARM1/PRMT-4, and JBP1/PRMT-5. Abramovich et al. (1997) Embo J, 16, 260-6; Chen et al. (1999) Science, 284, 2174-7; Katsanis et al. (1997) Mamm Genome, 8, 526-9; Lin et al. (1996) J Biol Chem, 271, 15034-44; Scott et al. (1998) Genomics, 48, 330-40; Tang et al. (1998) J Biol Chem, 273, 16935-45. A common characteristic of this family of enzymes is an S-adenosyl methionine (AdoMet) binding motif, related to the motif found in nucleic acid methyltransferases and small molecule methyltransferases that use AdoMet as a methyl donor. Kagan and Clarke (1994) Arch Biochem Biophys, 310, 417-27.

PRMT-2 was identified by exon trapping in human chromosome 21q.22.3 during EST searches. Katsanis et al. (1997) Mamm Genome, 8, 526-9; Scott et al. (1998) Genomics, 48, 330-40. PRMT-2 is the most distal gene on human chromosome 21q. Cole et al. (1998) Genomics, 50, 109-11. The biological function of PRMT-2, however, is not well understood.

Thus, while some speculation exists as to the functional significance of protein-arginine methyltransferases, the functional significance of protein-arginine methyltransferases, particularly PRMT-2, in vivo is largely unknown. Therefore, a need exists for identifying the functions of PRMT-2 and for methods for modulating those functions.

### SUMMARY OF THE INVENTION

The invention is directed to compositions and methods that involve modulating PRMT-2 activity or expression. In some embodiments, the methods involve directly modulating PRMT-2 activity or expression. In other embodiments, the invention provides methods involving modulating the activity or expression of PRMT-2 so that other cellular factors are influenced or modulated. For example, the activity of E2F, NFκB and STAT3 can be modulated by modulating the activity or expression of PRMT-2.

Thus, one aspect of the invention is a method for modulating NFκB or E2F1 activity in a mammal that comprises administering to the mammal a PRMT-2 polypeptide or a PRMT-2 nucleic acid that encodes a PRMT-2 polypeptide. The PRMT-2 polypeptide can, for example, have sequences SEQ ID NO:2, 3 or 6. The PRMT-2 nucleic acid can, for example, have SEQ ID NO:1. In some embodiments, the NFκB or E2F1 activity can be modulated to treat a disease or condition. For example, in some embodiments, increased PRMT-2 activity or expression can inhibit NFκB-related or E2F1-related functions. Examples of diseases or conditions that can be treated by modulating PRMT-2 activity or expression can therefore include inflammations, allergies, cancers, HIV infections, allograft rejections, adult respiratory distress syndrome, asthma, vasculitis, or vascular restenosis.

Another aspect of the invention is a method for inhibiting Protein Arginine N-Methyltransferase-2 activity or expression in a mammal that comprises administering to the mammal an antibody or nucleic acid that can inhibit the activity or expression of Protein Arginine N-Methyltransferase-2. The nucleic acid that can inhibit the activity or expression of PRMT-2 can, for example, be an antisense nucleic acid, a siRNA or a ribozyme that is selectively hybridizable under physiological conditions to an RNA derived from a DNA comprising SEQ ID NO:1. In some embodiments, the Protein Arginine N-Methyltransferase-2 expression is modulated to treat a disease or condition. Examples of diseases or conditions that can be treated by inhibiting PRMT-2 activity or expression include obesity, diabetes, hyperlipidemia, insulin insensitivity, and the like.

Another aspect of the invention is a method for modulating STAT3 activity in a mammal that comprises administering to the mammal a siRNA that is selectively hybridizable under stringent conditions to an RNA derived from a DNA comprising SEQ ID NO:1. STAT3 activity can be modulated to treat a disease or condition. Examples of diseases or conditions that can be treated in this manner include obesity, diabetes, hyperlipidemia, insulin insensitivity and the like.

Another aspect of the invention is a method for inhibiting transcription from an HIV-1 LTR in a mammal that comprises administering to the mammal an effective amount of a Protein Arginine N-Methyltransferase-2 polypeptide comprising SEQ ID NO:2, 3 or 6 and/or administering to the mammal an effective amount of a Protein Arginine N-Methyltransferase-2 nucleic acid, for example, a nucleic acid comprising SEQ ID NO:1.

Another aspect of the invention is a method for inhibiting transcription from an HIV-1 LTR in a mammalian cell that comprises contacting the mammalian cell with a Protein Arginine N-Methyltransferase-2 polypeptide comprising SEQ ID NO:2, 3 or 6 and/or contacting the mammalian cell with an effective amount of a Protein Arginine N-Methyltransferase-2 nucleic acid, for example, a nucleic acid comprising SEQ ID NO:1.

Another aspect of the invention is a method for inhibiting E2F1 transcriptional activity in a mammal that comprises administering to the mammal an effective amount of a Protein Arginine N-Methyltransferase-2 polypeptide comprising SEQ ID NO:2, 3 or 6 and/or administering to the mammal an effective amount of a Protein Arginine N-Methyltransferase-2 nucleic acid, for example, a nucleic acid comprising SEQ ID NO:1.

Another aspect of the invention is a method for inhibiting E2F1 transcriptional activity in a mammalian cell that comprises contacting the mammalian cell with a Protein Arginine N-Methyltransferase-2 polypeptide comprising SEQ ID NO:2, 3 or 6 and/or contacting the mammalian cell with an effective amount of a Protein Arginine N-Methyltransferase-2 nucleic acid, for example, a nucleic acid comprising SEQ ID NO:1.

Another aspect of the invention is a method for identifying a test agent that can modulate Protein Arginine N-Methyltransferase-2 expression in a cell comprising contacting the cell with a test agent and observing whether expression of a nucleic acid comprising SEQ ID NO:1 is modulated relative to expression of a nucleic acid comprising SEQ ID NO:1 in a cell that was not contacted with the test agent.

Another aspect of the invention is a method for identifying a test agent that can modulate Protein Arginine N-Methyltransferase-2 activity in a test cell comprising contacting the test cell with a test agent and observing whether Protein Arginine N-Methyltransferase-2 activity is modulated relative to Protein Arginine N-Methyltransferase-2 activity in a control cell that was not contacted with the test agent.

Another aspect of the invention is a method for identifying a test agent that can modulate Protein Arginine N-Methyltransferase-2 activity in a test cell comprising contacting the test cell with a test agent and observing whether NFκB activity is modulated relative to NFκB activity in a control cell that was not contacted with the test agent. The test cell can be, for example, a cancer cell, an immune cell or a cultured cell that has been exposed to an interleukin or a cytokine to induce an inflammatory response.

Thus, in some embodiments, the invention provides methods for treating or preventing diseases such as, for example, inflammation, allergies, cancer, obesity, diabetes, hyperlipidemia, adult respiratory distress syndrome (ARDS), asthma, allograft rejection, vasculitis, and vascular restenosis, as well as other conditions that are typically responsive to NFκB or E2F1 modulation, or that are responsive to methylated STAT3. Such methods can involve use of agents that inhibit PRMT-2 expression, use of agents that inhibit PRMT-2 activity, use of gene therapy to modulate or alter PRMT-2 expression, use of anti-PRMT-2 antibodies or use of siRNA or anti-sense nucleic acids that bind to PRMT-2 RNA.

### DESCRIPTION OF THE FIGURES

FIG. 1A-F illustrates that PRMT2 inhibits HIV-1 transcription in contrast to other arginine methyltransferases and that such inhibition by PRMT2 requires its methyltransferase domain. FIG. 1A shows that human PRMT1, 2 and 3 structures share a core arginine methyltransferase region, composed of an Ado-Met binding domain (striped central area) and divergent C-terminal domain (open area at the C-terminus). FIG. 1B shows that PRMT2 inhibits transcription from the HIV-1 LTR while related methyltransferases do not. 293T cells were transfected with 100 ng of 5 κB luciferase reporter and 1 μg of methyltransferase expression constructs as indicated, and luciferase activity was measured as described in Example 1. FIG. 1C illustrates expression of PRMT1, PRMT2 and PRMT3 in the 293T cells by western blot analysis of 15 μg of protein from each extract. Proteins were separated on a 4-15% polyacrylamide gel and transferred to a PVDF membrane. Western blotting was performed with a mouse anti-HA antibody. FIG. 1D provides a schematic representation of PRMT2 and PRMT2 mutants. Amino acids 141-144 (ILDV, SEQ ID NO:5) represent the Ado-Met consensus site in all PRMT family members. The Ado-Met consensus site in PRMT2 was mutated and replaced by four alanines (PRMT2-4A). PRMT2-A is an alternative splice variant of PRMT2 and lacks the divergent COOH-terminus found in other PRMT family members. PRMT2-N contains the first 95 amino acids of PRMT2. FIG. 1E shows that deletion of the methyltransferase domain abolishes PRMT2 inhibition of transcription. 293T cells were transfected with 1 μg of HA-tagged PRMT2, PRMT2-A, PRMT2-4A and PRMT2-N and 100 ng of 5 κB luciferase reporter. Cell lysates were collected 48 hours after transfection and luciferase activity was assayed. FIG. 1F illustrates expression of mutant PRMT2 proteins by western blot analysis of 15 μg of protein from each cell extract. Proteins were separated on a 4-15% polyacrylamide gel and transferred to a PVDF membrane. Western blotting was performed with a mouse anti-HA antibody.

FIG. 2A-E shows that transcriptional inhibition by PRMT2 is KB-dependent and IKK-2 or p65-induced NF-κB activation is blocked by PRMT2. FIG. 2A shows that transcriptional inhibition by PRMT2 is promoter specific using a CAT reporter assay and illustrates the effect of PRMT2 on HIV and other enhancers. 293 cells were transfected with 1 μg of the indicated reporter plasmids and 5 μg of either PRMT2-A or pVR1012 plasmids. Cells were harvested 36 hours post-transfection, and CAT assays were performed. Data shown are the mean (±SEM)-fold inhibition, in the presence of PRMT2-A, over a vector control of 3 independent experiments. A statistically significant effect of PRMT2-A on the HIV-1 promoter was noted at 5 μg (*p<0.01, compared to vector control, Student's t-test). PRMT2-A did not significantly inhibit transcription from the other promoters tested. FIG. 2B shows that PRMT2 inhibits gene expression in a κB-dependent manner. 293T human embryonic kidney cells were transfected with HIV-luciferase reporter (WT) or ΔκB-luciferase reporter (ΔκB) and PRMT2 or control vector plasmid. 24 hrs after transfection, cells were treated with vehicle (left panel) or with TNF-α (20 ng/ml; middle panel) or PMA (10 ng/ml; right panel) as indicated, and luciferase activity was measured 48 hrs post transfection. FIGS. 2C and 2D shows that PRMT2 inhibits expression of endogenous class I MHC, an NF-κB-dependent endogenous gene. 293T cells were transfected with HA-PRMT2 or HA-PRMT2-N. Transfected cells were detached after 48 hrs and analyzed by flow cytometry for MHC Class I and CD9 in HA-positive cells in PRMT2 (FIG. 2C) or PRMT2-N (FIG. 2D) transfected cells. FIG. 2E shows that NF-κB induction by IKK2 or p65 is efficiently is blocked by PRMT2. 293T cells were transfected with plasmids as indicated where IKK2 or p65 was added as shown, and the κB luciferase reporter gene was used to monitor NF-κB activity 48 hrs after transfection. Values are expressed as fold stimulation compared to the control vector.

FIG. 3A-C illustrates that PRMT2 does not interfere with p50/p65 dimerization or DNA binding. FIG. 3A shows that PRMT2 does not alter p65 expression or localization. 10 μg of cytoplasmic (CE) or nuclear (NE) extract from 293 cells transfected with vector and PRMT2 expression vectors were subjected to 4-15% SDS-PAGE and transferred to PVDF. The membrane was probed with an antibody to RelA (p65). FIG. 3B illustrates the effect of PRMT2 on NF-κB DNA binding, as assayed by analyzing the DNA binding activity of nuclear extracts from 293 cells cotransfected with NF-κB1 (p50)/RelA (p65) and PRMT2 or PRMT2-N expression vectors (lanes 1, 2, 3; left panel). PRMT2 inhibited p50/p65 DNA binding in a dose-dependent manner (lanes 5, 6; middle panel), and the shifted complex contained p50/p65 (lanes 8, 9; right panel). 36 hours after transfection, nuclear extracts were made and analyzed by EMSA with a ³²P-labeled double-stranded oligonucleotide containing the κB. NF-κB DNA binding activity was measured from nuclear extracts from 293 cells cotransfected with NF-κB1/RelA and vector control (5 μg, lane 4) or increasing amounts (2.5 and 5 μg, lanes 5, 6) of PRMT2 expression vector. EMSAs were performed as before, but antibodies to NF-κB were included in the reaction to confirm the nature of the retarded complexes. The complex is super-shifted by both p50 and p65 antibodies, confirming its identity as NF-κB (lanes 7-9). FIG. 3C shows that PRMT2 does not inhibit NF-κB DNA binding (left panel). Increasing amounts of GST (lanes 11-13) or GST-PRMT2 (lanes 14-16) were added to p65/p50 transfected 293 extracts, prior to the addition of the labeled probe to the reaction mix. EMSAs were carried out as before. No inhibition of NF-κB DNA binding was seen in the presence of GST-PRMT2. PRMT2 does not disrupt p50/p65 complex formation (right panel, lanes 17, 18). Immunoprecipitations were carried out from PRMT2 or PRMT2-N transfected 293 whole cell extracts, using a p65 antibody. p50 coimmunoprecipitated with p65 was detected by Western blotting using an anti-p50 antibody. No difference was detected in the amount of p50 brought down in the presence of PRMT2 or PRMT2-N, suggesting that PRMT2 does not disrupt p50/p65 complex formation.

FIG. 4A-B shows that PRMT2 promotes nuclear accumulation of IκB-α. FIG. 4A shows that more IκB-α is present in cellular nuclei when functional PRMT2 is present than when a truncated, non-functional mutant PRMT2-N is present. Cells were stimulated with TNF-α (200 U/ml) 24 hours after transfection and harvested at 36 hours. 10 μg of cytoplasmic extracts were resolved by 4-15% SDS-PAGE and transferred to PVDF. Immunoblotting was done with an anti-IκB-α antibody. The membrane was then stripped and reprobed using an antibody to tubulin. Cytoplasmic IκB-α levels remain unchanged in the presence of PRMT2 or PRMT2-N (lanes 1, 2). Increased IκB-α protein levels were observed in nuclear extracts from PRMT2-transfected cells (lane 3). Little or no IκB-α was seen in nuclear extracts from PRMT2-N transfected cells (lane 4). Blots were stripped and reprobed with antibodies to RelA (p65), p50 or Sp1 (middle and lower right panels). FIG. 4B graphically illustrates that nuclear IκB-α protein levels were increased ˜8-fold in PRMT2-transfected cells over the mutant control (see also FIG. 5). Film images were digitized using a scanner and the bands were quantified using Imagequant software. Data are expressed as the mean (±SEM) fold increase in nuclear IκB-α from 3 independent experiments.

FIG. 5A-E illustrates that PRMT2 associates with an endogenous IκB-α complex. FIG. 5A shows immunoprecipitation of endogenous PRMT2-IκB-α complex. NIH3T3 cell extracts (2 mg) were immunoprecipitated with agarose-conjugated antibodies against control IgG or IκB-α, resolved by 10% SDS-PAGE, and immunoblotted with antibody to PRMT2. FIG. 5B-E illustrates which regions of the IκB-α (FIGS. 5B and D) and PRMT2 (FIGS. 5C and E) proteins interact. A schematic representation of His-tagged IκB-α deletions is shown in FIG. 5B and a schematic representation of HA-tagged PRMT2 and it deletion mutants are shown in FIG. 5C. The IκB-α signal recognition domain (SRD) is the densely cross-hatched region near the N-terminus, ankyrin repeats are shown as striped regions in the middle and the PEST domain is shown as the large cross-hatched region at the C-terminus (FIG. 5C). FIG. 5C provides schematic diagrams of PRMT2 and its mutants, where the N-terminal domain is cross-hatched, arginine methyltransferase region, composed of an Ado-Met binding domain (striped middle region) and the divergent C-terminal domain (light, C-terminal region). FIG. 5D-E shows the results of mapping PRMT2-IκB-α interactions. To map IκB-α domains that interacted with PRMT2, 293 cells were transfected with HA-tagged PRMT2 (FIG. 5C) and His-tagged derivatives of the indicated IκB-α expression vectors (FIG. 5B) as indicated. 24 hrs later cells were harvested in cell lysis buffer and cell lysates were immunoprecipitated with agarose-conjugated antibody to His (IκB-α) (FIG. 5D, lanes 1-4) and HA (PRMT2) (FIG. 5D, lanes 5-6), fractionated by SDS-PAGE and analyzed by immunoblotting with antibody to HA to detect PRMT2 ((FIG. 5D lanes 1, 2 and lanes 5, 6) and His (FIG. 5D, lanes 3, 4). To map the region of PRMT2 that interacted with endogenous IκB-α and p65, 293 cells were transfected with HA-tagged PRMT2 (FIG. 5C) derivatives as indicated. Cells were harvested 24 hrs after transfection in cell lysis buffer, immunoprecipitated with agarose-conjugated antibody to HA to detect PRMT2 and derivatives (FIG. 5E, lanes 7-15), fractionated by SDS-PAGE and analyzed by immunoblotting with antibody to p65 (FIG. 5E, lanes 7-9), IκB-α (FIG. 5E, lanes 10-12) or HA (FIG. 5E, lanes 13-15).

FIG. 6A-D illustrates that the loss of NF-κB inhibition in PRMT2^{-/-} fibroblasts can be reversed by complementation through transfection of PRMT2, and dependence on LMB-sensitive nuclear export. FIG. 6A illustrates how PRMT2^{-/-} and PRMT2^{-/-} fibroblasts complemented with PRMT2 respond to the NF-κB promoter. PRMT2^{-/-} MEFs were transfected with a control vector or HA-tagged PRMT2 expression vector, and the NF-κB reporter construct (5×κB-Luciferase). PRMT2^{+/+} MEFs transfected with control vector and the NF-κB reporter (5×κB-Luciferase) served as the control. Thirty hours after transfection, cells with or without TNF-α treatment (1000 U/ml for 6 h) were harvested and analyzed by Dual-Luciferase Reporter Assay System (Promega). Renilla luciferase activity by PRL-TK was used as an internal standard to control transfection efficiency, and the fold increase in activity relative to unstimulated wild type cells is shown. Cross-hatched bars=unstimulated cells; open bar=TNF-stimulated cells. FIG. 6B shows that PRMT2^{-/-} MEFs have less nuclear IκB-α. 20 μg of cytoplasmic and nuclear extracts from PRMT2^{+/+} and PRMT2^{-/-} MEFs were resolved by 4-15% SDS-PAGE and transferred to PVDF. Immunoblotting was done with an anti-p65, p50 and anti-IκB-α antibody. FIG. 6C shows that PRMT2 affects nuclear export of IκB-α. PRMT2^{-/-} fibroblasts were transfected with an HA-tagged PRMT2 expression vector. 36 hrs after transfection, the cells were treated with TNF-α for 30 min. Media was removed and cells were incubated for an additional 30 minutes in the absence (top panel) or presence (bottom panel) of LMB. Cells were fixed, permeabilized and stained for IκBα (left panel; Alexa 488; green) and HA (PRMT2) (middle panel; Alexa 564; red). Overlay of IκBα and HA (PRMT2) staining is also shown (right panel). Nuclei are stained with DAPI (blue). FIG. 6D shows that leptomycin B (LMB) does not alter nuclear IκB-α in the presence of PRMT2. LMB promoted nuclear accumulation of IκBα in the absence of PRMT2, and transfection of PRMT2 exerted the same effect. Experiments were performed as described above. Quantification of nuclear IκBα in individual cells from several fields was done as follows: the outlines of cellular nuclei in a field were drawn using Leica confocal software. IκBα pixel intensity from the nucleus of each individual cell in the field was measured. The cells that had PRMT2 were distinguished by the presence of the HA tag (cross-hatched bars). The nuclei were identified by DAPI staining. For each condition, the data from 10 fields were compiled (approx 5-6 cells per field; 30% of the cells expressed PRMT2) and are presented on a graph, with p-values as indicated.

FIG. 7A-C shows that PRMT2^{-/-} cells are resistant to apoptosis, and this effect can be reversed by complementing PRMT2^{-/-} cells with PRMT2. FIG. 7A graphically illustrates that PRMT2 promotes TNF-α-induced apoptosis. Empty vector, mutant IκB-α (S32A/S36A, SR-IκB), or PRMT2 plasmids were co-transfected with CD2 into 293 cells. 24 hours after transfection, cells were stimulated with TNF-α (1000 U/ml) for 24 hours. Cells were stained with APC-labeled anti-CD2 antibody (BD Biosciences), annexin-V, and propidium iodide and analyzed by flow cytometry (FACS Caliber, BD Biosciences). The percentage of annexin-V positive and propidium iodide negative cells among CD2 positive cells are shown as the mean±standard deviation from three different experiments. FIG. 7B graphically illustrates the resistance of PRMT2^{-/-} MEFs to cell death after etoposide exposure. PRMT2^{+/+} and PRMT2^{-/-} MEFs (passage 4) were seeded at 2×10⁵ cells per well in 6-well plates. PRMT2^{-/-} MEFs were transfected with control or PRMT2 expression vector. PRMT2^{+/+} MEFs transfected with control vector served as the control. 24 hours after transfection, cells were stimulated with etoposide (100 μM) for 24 hours. Cells were treated with trypsin and stained with trypan blue (Invitrogen). Unstained surviving cells were counted with a hemocytometer. Cell death represents the percentage of treated cells that underwent apoptosis relative to untreated cells. Results are shown as the mean±S.E.M. of 3 independent experiments. FIG. 7C shows etoposide induced apoptosis in PRMT2^{+/+} compared to PRMT2^{-/-} MEFs. Bright field and fluorescent microscopy of PRMT2^{+/+} and PRMT2^{-/-} MEFs stained with FITC-Annexin V (20× magnification). Arrows indicate representative cells in light and dark fields.

FIG. 8A-C illustrate that PRMT2 interacts with retinoblastoma protein (RB). FIG. 8A shows that PRMT2 binds directly to RB in vitro. GST-RB and GST were bound to glutathione sepharose beads and incubated with S³⁵-labeled in vitro translated PRMT1, PRMT2, PRMT3, and PRMT4. Co-precipitated labeled PRMTs were analyzed by SDS-PAGE. As shown in FIG. 8B, Coomassie blue staining after SDS-PAGE verified that an equal amount of GST-RB and GST were loaded in each lane. FIG. 8C shows that PRMT2 co-immunoprecipitates with RB. HA-tagged PRMT2 (+) or control vectors (−) were transfected in 293 cells. Cell lysates were immunoprecipitated with rabbit RB antibodies or IgG and followed by Western blot analysis using mouse HA and RB antibodies (upper panel). A Western blot using mouse HA antibodies detects HA-tagged PRMT2 (lower panel).

FIG. 9A-B shows that PRMT2 interacts with retinoblastoma protein (RB) through Ado-Met binding domain. FIG. 9A provides the domain structure of PRMT2. The amino acid sequence of motif I, the AdoMet binding site, is shown. FIG. 9B provides a schematic diagram of PRMT2 deletion mutants used in the in vitro binding assays with RB (FIG. 9C). Direct protein-protein interactions between GST-RB and S³⁵-labeled PRMT2 mutants were analyzed by SDS-PAGE.

FIG. 10A-D shows that PRMT2 represses E2F transcriptional activity in RB-dependent manner. FIG. 10A provides a schematic diagram of a reporter construct (GAL4-driven luciferase reporter with a minimal TATA box) and an activator construct (SV40-driven E2F transcriptional activation domain fused to GAL4-DNA binding domain) used in E2F transcriptional assays. FIG. 10B shows that PRMT2 represses E2F activity in a dose dependant manner. HeLa cells were transiently transfected with 0.5 μg of reporter, 0.8 μg of activator and none, 0.6 μg or 1.8 μg of PRMT2 expression vector (pVR1012-PRMT2-HA). Luciferase activity was measured 36 hours later. FIG. 10C shows that the methyltransferase activity of PRMT2 is dispensable but that the Ado-Met binding domain is indispensable for E2F repression. U2OS cells were transiently transfected with reporter, activator, and/or 0.6 μg of the expression vectors. Vector=empty expression vector (pVR1012); Int-del=PRMT2(1-95&219-433). FIG. 10D shows that Rb is indispensable for the E2F repression by PRMT2. Rb negative Saos2 cell were transiently transfected with reporter, activator and/or the indicated amount of CMV-Rb and pVR1012-PRMT2-HA. In FIGS. 10B, C and D, the activity of promoter in the absence of activator and PRMT2 was normalized to a value of 1. The results are the mean±S.E.M. of four different experiments.

FIG. 11A show that E2F1, Rb, and PRMT2 form a complex. In FIG. 11A, Rb negative Saos2 cells were transfected with the indicated expression constructs, cell lysates were immunoprecipitated with an anti-E2F1 antibody, followed by Western blot analysis using an anti-HA antibody. Note that PRMT2 was co-immunoprecipitated with E2F1 in the presence of RB. A straight Western blot analysis for PRMT2 was used as a positive control. FIG. 11B illustrates expression levels of transfected PRMT2, Rb, and E2F1 by a Western blot analysis of the cell lysates.

FIG. 12A-D illustrates generation of PRMT2^{-/-} mice. FIG. 12A schematically diagrams the targeted disruption of the PRMT2 locus. The targeting vector was constructed to replace a portion of exon 4, 6 and all of exon 5 with a Neo^{R} gene in an antisense orientation. Point mutation for generating a stop codon (G119stop) is shown as a closed triangle. The probe for Southern blot screening and the PCR primers for genotyping are indicated. FIG. 12B provides PRMT2 genotyping by Southern blot. After EcoRI digestion, hybridization with the probe detects a 23 kb wild-type allele and a 5 kb mutant-allele. Probe position and the expected EcoRI-fragment sizes are indicated in FIG. 12A. FIG. 12C illustrates PRMT2 genotyping by PCR. Combined PCR reaction with sense primer (primer A at exon 4) and two antisense primer (primer B at exon 5; primer C at Neo^{R} gene) were used to detect the wild-type allele (190 bp) or mutant allele (280 bp), respectively. Primer positions are indicated in FIG. 12A. FIG. 12D provides a Northern blot demonstrating that PRMT2^{-/-} cells have no PRMT2 mRNA expression. RNA was harvested from hearts from PRMT2^{+/+} and PRMT2^{-/-} mice, and 2 μg of each poly A RNA was used for Northern hybridization with a probe derived from the entire coding region of mouse PRMT2 cDNA. Wild-type heart expresses an ˜2.4 kb message corresponding to PRMT2 mRNA. This mRNA is not present in the homozygous mutant heart.

FIG. 13A-E illustrate that an endogenous interaction occurs between PRMT2 and Rb. As illustrated in FIG. 13A, a mouse monoclonal antibody raised against PRMT2 (clone 5F8) detects PRMT2 only in PRMT2^{+/+} cells. PRMT2 transfected (+) and mock transfected (−) 293 cell lysates were analyzed by Western blot analysis using the monoclonal antibody. FIG. 13B shows that the anti-PRMT2 monoclonal antibody immunoprecipitates PRMT2. HA-tagged PRMT2 transfected (+) and mock transfected (−) 293 cell lysates were immunoprecipitated with the PRMT2 antibody or an anti-flag antibody, followed by a Western blot using rat anti-HA antibody (3F10). Input=Western blot analysis of 10% of immunoprecipitate input. FIG. 13C shows that the anti-PRMT2 monoclonal antibody detects endogenous PRMT2. Western blot analysis was performed on cell lysates from MEFs derived from PRMT2^{+/+} and PRMT2^{-/-} mice using the monoclonal antibody (upper panel). The blot was reprobed with an anti-actin antibody (lower panel). FIG. 13D illustrates an endogenous interaction between PRMT2 and Rb. Whole cell extracts from PRMT2^{+/+} or PRMT2^{-/-} MEFs were immunoprecipitated with the monoclonal antibody, followed by a Western blot using an anti-Rb antibody. FIG. 13E shows the expression levels of Rb in PRMT2^{+/+} and PRMT2^{-/-} MEFs as detected by a Western blot using an anti-Rb antibody. The Western blot is a positive control for Rb.

FIG. 14A-F illustrates that PRMT2^{-/-} MEFs show increased endogenous E2F activity and early S phase entry. FIG. 14A shows E2F-dependent reporter activity in PRMT2 MEFs. Asynchronously growing PRMT2^{+/+} and PRMT2^{-/-} MEFs at passage 3 were transfected with the E2F reporter (E2F4B-Luc). Luciferase activity was measured 30 hours later. The results are shown as the mean±S.E.M. of four different experiments. *p<0.01, PRMT2^{-/-} vs. PRMT2^{+/+} MEFs. FIGS. 14B-F show that PRMT2^{-/-} MEFs demonstrate early S phase entry. Asynchronously growing PRMT2^{+/+} and PRMT2^{-/-} MEFs at passage 3 were serum starved for 72 hours and then stimulated with 10% FBS. Cells were pulse labeled with BrdU (10 μM) for 1 hour, and the cells were harvested at 0 and 14 hours after serum release. BrdU incorporation and DNA-content were analyzed by flow cytometry. Representative cell sorting (two-color in the original) plots are shown in FIG. 14C-F. The percentages of BrdU positive cells are shown as the mean±S.E.M. of three different experiments in (FIG. 14B). *p<0.01, PRMT2^{-/-} vs. PRMT2^{+/+} MEFs.

FIG. 15A-F illustrate mutation of the PRMT2 locus to generate a PRMT2 knockout strain of mice. FIG. 15A illustrates targeted disruption of the PRMT2 locus. The targeting vector was constructed to replace a portion of exon 4, 6 and all of exon 5 with a NeoR gene in an antisense orientation. Point mutation for generating a stop codon (G119stop) is shown as a closed triangle. FIG. 15B shows PRMT2 genotyping by Southern blot. After EcoRI digestion, hybridization with PRMT2-specific probe detects a 23 kb wild-type allele and a 5 kb mutant-allele. Probe position and the expected EcoRI-fragment sizes are indicated in FIG. 15A. FIG. 15C shows PRMT2 genotyping by PCR. Combined PCR reaction with sense primer (primer A at exon 4) and two antisense primers (primer B at exon 5; primer C at NeoR gene) were used for detection of the wild-type allele (190 bp) or mutant allele (280 bp), respectively. Primer positions are indicated in FIG. 15A. FIG. 15D illustrates the relative expression levels of PRMT2 mRNA in various mouse tissues. Northern hybridization of poly A+ RNA from tissues to either full-length PRMT2 (upper panel), Stat3 (middle panel) or β-actin (lower panel) cDNA was performed as described in Example 3. FIG. 15E illustrates the relative expression levels of PRMT2 protein in various mouse tissues. Tissue extracts were isolated from wild-type mouse and were resolved by SDS-PAGE and immunoblotted with anti-PRMT2 antibody (upper panel) and anti-Stat3 antibody (lower panel). The control lane (293) were performed by using lysate from HEK293 cells transiently transfected with expression vectors encoding mouse PRMT2 and Stat3 cDNA. FIG. 15F shows that PRMT2 expression is absent in tissues from PRMT2^{-/-} mice. Tissue extracts were isolated from wild-type mouse and were verified by SDS-PAGE and immunoblotted with anti-PRMT2 antibody (upper panel) and anti-Stat3 antibody (lower panel).

FIG. 16A-B graphically illustrate body weight of PRMT2^{-/-} mice as a function of time and microscopic analysis of the PRMT2^{-/-} mice's liver. FIG. 16A shows growth curves of age-matched male wild-type (◯) and PRMT2^{-/-} (●) mice fed a standard chow diet for 30 weeks post-weaning. Values of body weight represent the mean±SEM of 6-12 mice for each genotype. FIG. 16B shows hematoxylin and eosin stained (upper panels) and PAS stained (lower panels) liver sections from 8-week-old wild-type (WT) and PRMT2^{-/-} (KO) mice. Original Magnification was ×400.

FIG. 17A-B graphically illustrate body weight gain and fad pad mass of high fat-fed PRMT2^{-/-} mice as a function of time. FIG. 17A shows body weight curves of age-matched male wild-type (◯), PRMT2+/− (▴) and PRMT2−/− (●) mice fed a high fat diet for 10 weeks. Values of body weight represent the mean±SEM of 7 mice for wild-type, 5 mice for PRMT2^{+/-} and 8 mice for PRMT2^{-/-} genotype. *P<0.05 vs. wild-type. **P<0.01 vs. wild-type. ***P<0.001 vs. wild-type. †P<0.05 vs. PRMT2^{+/-}. FIG. 17B shows fat pad and liver mass of age-matched male wild-type (open bars), PRMT2^{+/-} (striped bars) and PRMT2^{-/-} (cross-hattched bars) mice fed a high-fat diet. Mice were killed at the end of study, and the mass of individual fat pad depots was determined. Values of body weight represent the mean±SEM of 9-10 mice for each genotype. *P<0.05 vs. wild-type. **P<0.01 vs. wild-type. ***P<0.001 vs. wild-type. †P<0.05 vs. PRMT2^{+/-}.

FIG. 18 graphically illustrates leptin sensitivity of PRMT2^{-/-} mice. 12-16-week-old mice were injected with PBS followed by recombinant mouse leptin as described in Example 3. FIG. 18A illustrates the weight changes of leptin-treated wild-type (◯) and PRMT2^{-/-} (●) mice. Values represent the mean±SEM of eight mice. *P<0.05 vs. wild-type. **P<0.01 vs. wild-type. ***P<0.001 vs. wild-type. FIG. 18B shows the food intake of leptin-treated wild-type (open bars) and PRMT2−/− (cross-hatched) mice. Values are expressed as the percentage of food intake during PBS injection and represent mean±SEM of eight mice. *P<0.05 vs. wild-type.

FIG. 19A-B illustrate PRMT2 and Stat3 mRNA expression in mouse brain at the coronal level of the anterior and medial hypothalamus. FIG. 19A shows wild-type mouse brain (upper panel) and PRMT2−/− mouse brain (lower panel) sections after hybridization with a PRMT2-specific antisense riboprobe. Note that PRMT2 is highly expressed through the entire hypothalamus with particularly very high expression in the paraventricular hypothalamic, supraoptic arcuate and ventromedial hypothalamic nuclei. Extrahypothalamic areas that expressed PRMT2 include the pyramidal cell layer of the hippocampus and the thalamic paraventricular nucleus. Low and moderate expression levels were detected in the amygdaloid complex and cortical layers. Note the lack of hybridization signal in the thalamus and striatum. Significantly, substantially no hybridization signal was detected in PRMT2^{-/-} mouse brains. FIG. 19B shows wild-type mouse brain (upper panel) and PRMT2−/− mouse brain (lower panel) sections hybridized with a Stat3-specific antisense probe. Note that Stat3 is highly expressed in the paraventricular hypothalamic, ventromedial hypothalamic and arcuate nuclei of both wild-type and PRMT2^{-/-} mice. Arc: arcuate nuclei; VMH: ventromedial hypothalamic nuclei PT: paraventricular thalamic nuclei; PVH: parventricular hypothalamic nuclei; SON: supraoptic nuclei; pyrCA1-CA2: pyramidal cell layer of the hippocampus.

FIG. 20A-B shows methylation of Stat3 by PRMT2 in vitro. FIG. 20A shows electrophoretically separated reaction mixture of Flag-PRMT2 of Flag-PRMT2 mutant with GST-Stat3 and methyl donor S-adenosyl-1[methyl-³H]methionine ([³H]-AdoMet). The reactions were incubated for 1 hr at 4° C. and were terminated by addition of 3×SDS-loading buffer. The samples were subjected to SDS-PAGE in 4-15% Tris-HCl gradient gel, transferred to a poly(vinylidene difluoride) (PVDF) membrane, sprayed with En³hance and exposed to film. FIG. 20B shows methylation of GST, GST-Stat3 and GST-Stat3 Arg³¹→Ala³¹ by PRMT2. Wild-type and PRMT2^{-/-} MEF extracts were extracted as a PRMT2 enzyme source for the reaction. In vitro methylation reactions were performed by adding the PRMT2-containing immune complexes or the cell lysates to 1 μg of GST, GST-Stat3 and GST-Stat3 Arg³¹→Ala³¹ and 2 μCi of the methyl donor [³H]-AdoMet). The samples were analyzed with SDS-PAGE followed by autoradiography (upper panels). The positions of molecular weight markers are indicated at the right. The GST protein amount of each lane was shown by the Coomassie stained gel (lower panels).

FIG. 21A-B illustrates direct association of PRMT2 with Stat3 in vivo. FIG. 21A shows electrophoretically separated 293 cell lysates after immunoprecipitation and/or immunoblotting. The 293 cells were transiently transfected with expression vectors encoding mouse PRMT2-Flag and/or Stat3. After incubation for 24 hr, cells were lysed and samples were subjected to immunoprecipitation with anti-Flag antibody or co-precipitation with preimmune rabbit IgG followed by immunoblotting with anti-Stat3 antibody (upper panel). The control immunoblotting with anti-Flag antibody (second panel), anti-Stat3 antibody (third panel) and anti-β-actin antibody (lower panel) were performed by using same samples. FIG. 21B shows the effect of leptin on Stat3 methylation by PRMT2. Quiescent GT-1 cells were treated with or without recombinant mouse leptin (100 nM) for 10 min. Total cell lysates were subjected to immunoprecipitation with anti-Stat3 antibody or co-precipitation with preimmune rabbit IgG followed by immunoblotting with anti-PRMT2 antibody (upper panel) and anti-Stat3 antibody (lower panel).

FIG. 22A-D illustrate in vivo methylation of Stat3 by PRMT2. FIG. 22A endogenous methylation of STAT3 in wild-type PRMT-2-transfected cells (lane 2) and that such Stat3 methylation was increased when STAT3 was cotransfected into the cells (lane 3). 293 cells were transiently transfected with expression vectors encoding mouse PRMT2, PRMT2 lacking AdoMet binding domain and/or Stat3. After incubation for 24 hr, cells were lysed and samples were subjected to immunoprecipitation with anti-Stat3 antibody or co-precipitation with preimmune rabbit IgG followed by immunoblotting with anti-arginine (mono- and di-methyl) antibody (α-metR) (upper panel). The control immunoblots using anti-Stat3 antibody (second panel), anti-PRMT2 antibody (third panel) and anti-β-actin antibody (lower panel) were performed by using same samples. FIG. 22B shows the effect of leptin treatment on Stat3 methylation by PRMT2 inn GT-1 cells. Quiescent GT-1 cells were treated with or without recombinant mouse leptin (100 nM) for the indicated times. Total cell lysates were subjected to immunoprecipitation with anti-Stat3 antibody followed by immunoblotting with anti-arginine (mono- and di-methyl) (α-metR) (upper panel) and anti-Stat3 antibody (lower panel). FIG. 22C also illustrates the effect of leptin on Stat3 methylation by PRMT2. Quiescent GT-1 cells were treated with or without recombinant mouse leptin (100 nM) for 10 min. Cell lysates were then subjected to immunoprecipitation with anti-Stat3 antibody or co-precipitation with preimmune rabbit IgG followed by immunoblotting with anti-α-metR (upper panel) and anti-Stat3 antibody (lower panel). FIG. 22D illustrates the effects of leptin on Stat3 methylation by PRMT2 in vascular smooth muscle cells (VSMCS). Quiescent wild-type and PRMT2^{-/-} VSMCs were treated with or without recombinant mouse leptin (100 nM) for the indicated times. Total cell lysates were subjected to immunoprecipitation with anti-Stat3 antibody or co-precipitation with preimmune rabbit IgG followed by immunoblotting with anti-α-metR antibody (upper panel) and anti-Stat3 antibody (lower panel).

FIG. 23A-E illustrate the effects of PRMT2 knockout on leptin-induced Stat3 tyrosine phosphorylation. FIG. 23A shows Stat3 tyrosine phosphorylation in vascular smooth muscle cells. Quiescent wild-type and PRMT2^{-/-} VSMCs were treated with or without recombinant mouse leptin (100 nM) for the indicated times. Nuclear extracts were subjected to immunoblotting using anti-phospho-Stat3 [pStat3 (pY705)] antibody (upper panel) and anti-Stat3 antibody (lower panel). FIG. 23B graphically illustrates the amount of Stat3 phosphorylation in PRMT2^{+/+} and PRMT2^{-/-} cells. The pStat3 (pY705) and Stat3 signals from the X-ray films of the exposed blots were quantified by densitometry, and the amounts of phospho-Stat3 in wild-type (open bars) and PRMT2−/− (cross-hatched bars) cells were normalized to the amount of Stat3 present in each sample. Each bar in the graph represents the mean±SEM of the relative phosphorylation of Stat3 from the results of three independent experiments. *P<0.05 vs. wild-type. FIG. 23C that 30 minutes after leptin stimulation, tyrosine phosphorylated STAT3 remained localized within the nucleus of PRMT-2^{-/-} cells whereas by this time tyrosine phosphorylation of Stat3 had declined in the nucleus of wild-type cells. Quiescent wild-type and PRMT2^{-/-} VSMCs were treated with or without recombinant mouse leptin (100 nM) for the indicated times. Cells were subjected to immunocytochemistry with anti-pStat3 (pY705) antibody. Localization of tyrosine phosphorylated Stat3 was analyzed using a Nikon microscope. FIG. 23D shows phospho-Stat3 expression in hypothalamic sections from wild-type and PRMT2^{-/-} mice. Phospho-Stat3 immunoreactivities in hypothalamic sections from wild-type and PRMT2^{-/-} mice are shown under leptin-stimulated conditions as described in Example 3. Localization of tyrosine phosphorylated Stat3 was analyzed using a Nikon microscope. Arc: arcuate nuclei; VMH: ventromedial hypothalamic nuclei; 3V, Third ventricle. FIG. 23E illustrates the effects of a phosphatase inhibitor, o-vanadate, on Stat3 phosphorylation. Quiescent wild-type and PRMT2^{-/-} VSMCs were pretreated with or without o-vanadate (200 μM) for 15 min and then stimulated with recombinant mouse leptin (100 nM) for 30 min. Nuclear extracts were subjected to immunoblotting using anti-pStat3 (pY705) antibody (upper panel) and anti-Stat3 antibody (lower panel).

FIG. 24A-B graphically illustrate the effects of leptin on the expression of pro-opiomelanocortin (POMC; FIG. 24A) and neuropeptide Y (NPY; FIG. 24B) in the hypothalamuses of wild type and PRMT2^{-/-} mice. 12-16-week-old mice were injected with either PBS or recombinant mouse leptin as described in Example 3. Hypothalamic POMC and NPY mRNA was quantified by fluorescent Real-time PCR of RNA from 8-12 week-old wild-type (open bars) and PRMT2^{-/-} (cross-hatched bars) mice. Values are expressed as the percentage of each mRNA expression PBS-injected control and represent mean±SEM of five mice. *P<0.05 vs. wild-type.

FIG. 25A-B schematically illustrate some of the physiological events that occur in PRMT2^{+/+} and PRMT2^{-/-} cells.

### DETAILED DESCRIPTION OF THE INVENTION

The invention provides methods for modulating PRMT-2 activity and expression. Such methods are useful for treating a variety of conditions including inflammation, allergies, cancer, HIV-1 infection, obesity, diabetes, hyperlipidemia, insulin insensitivity, adult respiratory distress syndrome (ARDS), asthma, allograft rejection, vasculitis, and vascular restenosis, as well as other conditions that are typically responsive to modulating NFκB, EF2 or STAT3 activity. Also provided are methods for identifying agents that can modulate PRMT-2 activity or expression.

Definitions

Abbreviations: deoxyribonucleic acid (DNA), ribonucleic acid (RNA), Protein Arginine N-Methyltransferase 2 (PRMT-2).

The term “modulate” refers to an increase or decrease in PRMT-2 expression or activity. For example, modulation of PRMT-2 expression can refer to an increase or decrease in the production of mRNA that encodes PRMT-2. Modulation can also refer to an increase or decrease in translation of the mRNA that encodes PRMT-2 that results in an increase or decrease production of the PRMT-2 protein. Modulation can also refer to an increase or decrease in PRMT-2 enzymatic activity. PRMT-2 activators and PRMT-2 inhibitors modulate PRMT-2 expression and/or PRMT-2 activity. PRMT-2 inducers modulate PRMT-2 gene transcription and/or expression. PRMT-2 activity is the effect of the PRMT-2 protein in biological systems.

Protein Arginine N-Methyltransferases

Protein arginine methyltransferases (PRMTs) methylate arginine residues during post-translational modification of proteins. McBride, A. E. and Silver, P. A. (2001) Cell, 106, 5-8. The PRMT family consists of at least five members, including PRMT1, PRMT-2, PRMT3, CARM1/PRMT4, and JBP1/PRMT5. Abramovich et al. (1997) Embo J, 16, 260-6; Chen et al. (1999) Science, 284, 2174-7; Katsanis et al. (1997) Mamm Genome, 8, 526-9; Lin et al. (1996) J Biol Chem, 271, 15034-44; Scott et al. (1998) Genomics, 48, 330-40; Tang et al. (1998) J Biol Chem, 273, 16935-45.

One characteristic of this family of enzymes is an S-adenosyl methionine (AdoMet) binding motif, related to the motif found in nucleic acid and small molecular methyltransferases that use AdoMet as a methyl donor. Kagan and Clarke (1994) Arch Biochem Biophys, 310, 417-27. PRMTs have been implicated in various aspects of RNA processing and/or nucleocytoplasmic transport, receptor mediated signaling, and transcriptional regulation. Aleta et al. (1998) Trends in Biochemical Sciences, 23, 89-91; Chen et al. (1999) Science, 284, 2174-7; Koh et al. (2001) Journal of Biological Chemistry, 276, 1089-1098; Mowen et al. (2001) Cell, 104, 731-741. Recent results indicate that PRMTs can positively and negatively transcriptionally regulate some genes through cofactor methylation and/or histone methylation. Bauer et al. (2002) Embo Reports, 3, 39-44; Wang et al. (2001) Science, 293, 853-857; Xu et al. (2001) Science, 294, 2507-2511. For example, PRMT was found to function as a co-activator for the estrogen-dependent transcription. Qi (2002) Journal of Biological Chemistry, 277, 28624-28630.

Prior to the invention, the biological function of PRMT-2 was not fully understood.

Protein Arginine N-Methyltransferase 2 (PRMT-2)

PRMT-2 was identified by exon trapping in human chromosome 21q.22.3 during EST searches. Katsanis et al. (1997) Mamm Genome, 8, 526-9; Scott et al. (1998) Genomics, 48, 330-40. PRMT-2 is also the most telomeric gene on human chromosome 21q. Cole et al. (1998) Genomics, 50, 109-11. A genomic sequence for human PRMT-2 can be found in the NCBI database at accession number AP001761 (gi: 7768688).

A nucleotide sequence for human PRMT-2 can also be found in the NCBI database at accession number U80213 (gi: 1857418). See website at ncbi.nlm.nih.gov. This PRMT-2 nucleotide sequence is provided below as SEQ ID NO:1.

| | | | | |
|---|---|---|---|---|
| 1 | CACTGCGCTT | GCGCGGGTTG | AGGGCGGTGG | CTCAGTCTCC |
| 41 | TGGAAAGGAC | CGTCCACCCC | TCCGCGCTGG | CGGTGTGGAC |
| 81 | GCGGAACTCA | GCGGAGAAAC | GCGATTGAGA | AATGGAAAAG |
| 121 | AAAATGAAAT | AAATCAGCAG | TTATGAGGCA | GAGCCTAAGA |
| 161 | GAACTATGGC | AACATCACGT | GACTGTCCCA | GAAGTGAATC |
| 201 | GCAGGGAGAA | GAGCCTGCTG | AGTGCAGTGA | GGCGGGTCTC |
| 241 | CTGCAGGAGG | GAGTACAGCC | AGAGGAGTTT | GTGGCCATCG |
| 281 | CGGACTACGC | TGCCACCGAT | GAGACCCAGC | TCAGTTTTTT |
| 321 | GAGAGGAGAA | AAAATTCTTA | TCCTGAGACA | AACCACTGCA |
| 361 | GATTGGTGGT | GGGGTGAGCG | TGCGGGCTGC | TGTGGGTACA |
| 401 | TTCCGGCAAA | CCATGTGGGG | AAGCACGTGG | ATGAGTACGA |
| 441 | CCCCGAGGAC | ACGTGGCAGG | ATGAAGAGTA | CTTCGGCAGC |
| 481 | TATGGAACTC | TGAAACTCCA | CTTGGAGATG | TTGGCAGACC |
| 521 | AGCCACGAAC | AACTAAATAC | CACAGTGTCA | TCCTGCAGAA |
| 561 | TAAAGAATCC | CTGACGGATA | AAGTCATCCT | GGACGTGGGC |
| 601 | TGTGGGACTG | GGATCATCAG | TCTCTTCTGT | GCACACTATG |
| 641 | CGCGGCCTAG | AGCGGTGTAC | GCGGTGGAGG | CCAGTGAGAT |
| 681 | GGCACAGCAC | ACGGGGCAGC | TGGTCCTGCA | GAACGGCTTT |
| 721 | GCTGACATCA | TCACCGTGTA | CCAGCAGAAG | GTGGAGGATG |
| 761 | TGGTGCTGCC | CGAGAAGGTG | GACGTGCTGG | TGTCTGAGTG |
| 801 | GATGGGGACC | TGCCTGCTGT | TTGAGTTCAT | GATCGAGTCC |
| 841 | ATCCTGTATG | CCCGGGATGC | CTGGCTGAAG | GAGGACGGGG |
| 881 | TCATTTGGCC | CACCATGGCT | GCGTTGCACC | TTGTGCCCTG |
| 921 | CAGTGCTGAT | AGGATTATCG | TAGCCAAGGT | GCTCTTCTGG |
| 961 | GACAACGCGT | ACGAGTTCAA | CCTCAGCGCT | CTGAAATCTT |
| 1001 | TAGCAGTTAA | GGAGTTTTTT | TCAAAGCCCA | AGTATAACCA |
| 1041 | CATTTTGAAA | CCAGAAGACT | GTCTCTCTGA | ACCGTGCACT |
| 1081 | ATATTGCAGT | TGGACATGAG | AACCGTGCAA | ATTTCTGATC |
| 1121 | TAGAGACCCT | GAGGGGCGAG | CTGCGCTTCG | ACATCAGGAA |
| 1161 | GGCGGGGACC | CTGCACGGCT | TCACGGCCTG | GTTTAGCGTC |
| 1201 | CACTTCCAGA | GCCTGCAGGA | GGGGCAGCCG | CCGCAGGTGC |
| 1241 | TCAGCACGGG | GCCCTTCCAC | CCCACCACAC | ACTGGAAGCA |
| 1281 | GACGCTGTTC | ATGATGGACG | ACCCAGTCCC | TGTCCATACA |
| 1321 | GGAGACGTGG | TCACGGGTTC | AGTTGTGTTG | CAGAGAAACC |
| 1361 | CAGTGTGGAG | AAGGCACATG | TCTGTGGCTC | TGAGCTGGGC |
| 1401 | TGTCACTTCC | AGACAAGACC | CCACATCTCA | AAAAGTTGGA |
| 1441 | GAAAAAGTCT | TCCCCATCTG | GAGATGACAG | TTGATGCTTT |
| 1481 | ATTTGGAAAG | CAGTGTGCAT | ATCTTGAGGG | GTGATGAACA |
| 1521 | CAAGCAAACC | AAGTTGCACC | TGGCTTCTGC | ACACTCCTGC |
| 1561 | GAAAGTCGGT | GAACATTCAC | TCCACATTGA | CCCCTCCCTA |
| 1601 | GCCTGGCAGG | TGACGTCAGG | GTCCTTCACA | GACAAACACG |
| 1641 | CTTGGGCTCG | GCAGGAGCTG | CCGTGGCCAC | CCCCGCTGCC |
| 1681 | CAGTGTCTGC | CCTCTAGAAG | TAGGCTGTGT | TTCCAGGTGT |
| 1721 | TCACCCGTGG | TGCCCACAGT | GCCGACCCGT | GGCTGGGTCG |
| 1761 | GAGCTCCATG | TTCCTAAGCT | AGGTCTAGGT | CTACACTCCT |
| 1801 | AGGACGCACG | CATATCAGCC | CGTGTACCCT | GTGACAGTGA |
| 1841 | CTGTCCCCAC | CTCCTGTGTT | AGTGGTGCCC | TTACTGCCGT |
| 1881 | CGCTCATCCA | CTCGTGTGGG | ACGTAGGATT | GCACAGGGCT |
| 1921 | GTGCCAGTGG | CGTGTAGGGA | ACACTGCCCT | GGCTCAGCGT |
| 1961 | GCGAGCTAAG | GTGGCGATGT | ATGCGATGGG | ACTCTGCATG |
| 2001 | GGATAGTACA | GTTGTGTAGA | CGTCTTCCAA | ATAAATTATG |
| 2041 | TGTTGGTGCC | ATCGCACATG | CTCAATAAAT | ATTTTAAATG |
| 2081 | AGTGAAAAAA | AAA | | |

An amino acid sequence for human PRMT-2 can be found in the NCBI database at accession number P55345 (gi: 2499805). See website at ncbi.nlm.nih.gov. This PRMT-2 sequence is provided below as SEQ ID NO:2.

| | | | | |
|---|---|---|---|---|
| 1 | MATSGDCPRS | ESQGEEPAEC | SEAGLLQEGV | QPEEFVAIAD |
| 41 | YAATDETQLS | FLRGEKILIL | RQTTADWWWG | ERAGCCGYIP |
| 81 | ANHVGKHVDE | YDPEDTWQDE | EYFGSYGTLK | LHLEMLADQP |
| 121 | RTTKYHSVIL | QNKESLTDKV | ILDVGCGTGI | ISLFCAHYAR |
| 161 | PRAVYAVEAS | EMAQHTGQLV | LQNGFADIIT | VYQQKVEDVV |
| 201 | LPEKVDVLVS | EWMGTCLLFE | FMIESILYAR | DAWLKEDGVI |
| 241 | WPTMAALHLV | PCSADKDYRS | KVLFWDNAYE | FNLSALKSLA |
| 281 | VKEFFSKPKY | NHILKPEDCL | SEPCTILQLD | MRTVQISDLE |
| 321 | TLRGELRFDI | RKAGTLHGFT | AWFSVHFQSL | QEGQPPQVLS |
| 361 | TGPFHPTTHW | KQTLFMMDDP | VPVHTGDVVT | GSVVLQRNPV |
| 401 | WRRHMSVALS | WAVTSRQDPT | SQKVGEKVFP | IWR |

The invention also provides a PRMT-2-A mutant polypeptide that is an alternatively spliced form of PRMT-2 found in the expressed sequence tag (EST) database. This isoform contains the first 218 amino acids of PRMT-2 and differs from full length PRMT-2 by the absence of the less conserved COOH-terminal domain. The amino acid sequence for the PRMT-2-A polypeptide is provided below as SEQ ID NO:3.

| | | | | |
|---|---|---|---|---|
| 1 | MATSGDCPRS | ESQGEEPAEC | SEAGLLQEGV | QPEEFVAIAD |
| 41 | YAATDETQLS | FLRGEKILIL | RQTTADWWWG | ERAGCCGYIP |
| 81 | ANHVGKHVDE | YDPEDTWQDE | EYFGSYGTLK | LHLEMLADQP |
| 121 | RTTKYHSVIL | QNKESLTDKV | ILDVGCGTGI | ISLFCAHYAR |
| 161 | PRAVYAVEAS | EMAQHTGQLV | LQNGFADIIT | VYQQKVEDVV |
| 201 | LPEKVDVLVS | EWMGTCLL | | |

The invention also provides a PRMT-2-N polypeptide that was generated by introducing a stop codon after amino acid 95 of PRMT-2. The amino acid sequence for the PRMT-2-N polypeptide is provided below as SEQ ID NO:4.

| | | | | |
|---|---|---|---|---|
| 1 | MATSGDCPRS | ESQGEEPAEC | SEAGLLQEGV | QPEEFVAIAD |
| 41 | YAATDETQLS | FLRGEKILIL | RQTTADWWWG | ERAGCCGYIP |
| 81 | ANHVGKHVDE | YDPED | | |

The invention further provides a PRMT-2-4A mutant polypeptide in which amino acids 141-144 (₁₄₁ILDV₁₄₄, SEQ ID NO:5) in the Ado-Met domain of PRMT-2 have been altered to four consecutive alanines. The PRMT-2-4A has substantially no methyltransferase activity but is otherwise structurally similar to the PRMT-2 polypeptide. The amino acid sequence for the PRMT-2-4A polypeptide, with the four substituted alanines is provided below as SEQ ID NO:6.

| | | | | |
|---|---|---|---|---|
| 1 | MATSGDCPRS | ESQGEEPAEC | SEAGLLQEGV | QPEEFVAIAD |
| 41 | YAATDETQLS | FLRGEKILIL | RQTTADWWWG | ERAGCCGYIP |
| 81 | ANHVGKHVDE | YDPEDTWQDE | EYFGSYGTLK | LHLEMLADQP |
| 121 | RTTKYHSVIL | QNKESLTDKV | AAAAGCGTGI | ISLFCAHYAR |
| 161 | PRAVYAVEAS | EMAQHTGQLV | LQNGFADIIT | VYQQKVEDVV |
| 201 | LPEKVDVLVS | EWMGTCLLFE | FMIESILYAR | DAWLKEDGVI |
| 241 | WPTMAALHLV | PCSADKDYRS | KVLFWDNAYE | FNLSALKSLA |
| 281 | VKEFFSKPKY | NHILKPEDCL | SEPCTILQLD | MRTVQISDLE |
| 321 | TLRGELRFDI | RKAGTLHGFT | AWFSVHFQSL | QEGQPPQVLS |
| 361 | TGPFHPTTHW | KQTLFMMDDP | VPVHTGDVVT | GSVVLQRNPV |
| 401 | WRRHMSVALS | WAVTSRQDPT | SQKVGEKVFP | IWR |

These and related PRMT-2 polypeptides (for example, variants and derivatives of these polypeptides) can be used in the methods of the invention.

Nuclear Factor Kappa B (NFκB)

According to the invention, PRMT-2 inhibits NFκB function, including transcription mediated by NFκB. While not wishing to be limited to a particular mechanism, it appears that PRMT-2 provides such inhibition by causing nuclear accumulation of IκB, which concomitantly decreases binding of NFκB to nuclear DNA. Mutation or deletion of the conserved S-adenosyl methionine binding domain of PRMT-2 abolishes its activity to inhibit transcription by NFκB.

Upon cellular exposure to stimuli, such as an infection or stress, the NFκB transcription factor triggers gene expression. For example, when a cell is subjected to an infection, within minutes NFκB triggers vasodilation and infiltration of macrophages. Under normal circumstances, the NFκB transcription factor is tightly regulated to allow an appropriate and rapid response to infection or stress while preventing an inappropriate inflammation from a false trigger. Misregulation of NFκB, however, can cause uncontrolled expression of inflammation-causing genes and contributes to the pathogenesis of a number of diseases including rheumatoid arthritis, bronchial asthma, inflammatory bowel disease, septic shock, adult respiratory distress syndrome, and transplant rejection. It also plays a role in autoimmune diseases including diabetes. In rheumatoid arthritis, for example, activation of NFκB causes release of inflammatory mediators including prostaglandins, thromboxanes, and leukotrienes. Roshak et al. (1996) J. Biol. Chem. 271:31496-31501. Moreover, NFκB leads to release of adhesion molecules that may allow the leukocytes to interact with synoviocytes, and NFκB stimulates production of IL-6, IL-8 and GM-CSF. Sakurada et al. (1996) Int. Immunol. 8:1483-1493. Finally, NFκB induces further production of TNF-α and IL-1, leading to a feedback loop that amplifies the inflammation response.

Activation of NFκB is also associated with cancer. For example, virally encoded gene products, protein X from hepatitis B and tax from human T-cell leukemia virus activate NFκB and other transcription factors and cause improper cell proliferation. Gilmore et al. (1996) Oncogene. 13:1367-1378; Mosialos (1997) Sem. Cancer Biol. 8:121-129. In addition, TNF and NFκB contribute to skeletal muscle decay known as cachexia (Guttridge et al. (2000) Science. 289:2363-2366), which accounts for one third of cancer mortalities with inflammatory origin.

Moreover, the HIV-1 long terminal repeat (LTR) contains two highly conserved κB-binding sites that play an important regulatory role in HIV-1 gene expression. Nabel, G. & Baltimore, D. Nature 326, 711-713 (1987). As illustrated herein, transfection of PRMT-2 nucleic acids into cells that contain transcriptionally active HIV-1 nucleic acids, inhibits HIV-1 transcription. Thus, while not wishing to be limited to a particular mechanism it appears that PRMT-2 inhibition of HIV-1 transcription may operate through NF-κB and the κB binding site(s) on the HIV-1 LTR.

The invention therefore provides methods for treating diseases related to inappropriate NFκB expression or activity that involve modulating PRMT-2 expression or activity. In some embodiments, modulating the activity or expression of PRMT-2 involves administering an effective amount of PRMT-2 polypeptides or nucleic acids to a mammal or contacting a cell with an effective amount PRMT-2 polypeptides or nucleic acids. Addition of PRMT-2 polypeptides or nucleic acids can inhibit NFκB expression or activity. In other embodiments, modulating the activity or expression of PRMT-2 involves administering an effective amount of an agent that can inhibit PRMT-2 activity or expression. Such agents are described in more detail hereinbelow.

Diseases that involve inappropriate NFκB expression or activity, and that can be treated with the methods of the invention include, for example, adult respiratory distress syndrome (ARDS), allergies, allograft rejection, autoimmune diseases, bronchial asthma, cancer, diabetes, inflammation, inflammatory bowel disease, HIV-1 infection, rheumatoid arthritis, septic shock, transplant rejection, vasculitis, vascular restenosis as well as other conditions that are typically responsive to inhibition of NFκB.

PRMT-2 also renders cells susceptible to apoptosis by cytokines or cytotoxic drugs, possibly due to its effects on NFκB. Moreover, as shown by the inventors, embryonic fibroblasts from PRMT-2 genetic knockout mice have increased NFκB activity and decreased susceptibility to apoptosis compared to wild type cells. The invention therefore provides methods for modulating apoptosis by modulating PRMT-2 expression or activity. For example, the invention provides a method for increasing a cell's susceptibility to apoptosis that involves modulating PRMT-2 expression or activity. In some embodiments, modulating the activity or expression of PRMT-2 involves administering an effective amount of PRMT-2 polypeptides or nucleic acids to a mammal or contacting a cell with an effective amount PRMT-2 polypeptides or nucleic acids. Addition of PRMT-2 polypeptides or nucleic acids can inhibit NFκB expression or activity and increase the susceptibility of a cell to apoptosis. In other embodiments, modulating the activity or expression of PRMT-2 involves administering an effective amount of an agent that can inhibit PRMT-2 activity or expression. Such agents are described in more detail hereinbelow.

The transcription factor NFκB is constitutively expressed in the cytoplasm of cells. Induction of gene transcription by NFκB-like proteins results from post-translational modification permitting translocation of the preformed transcription factor from the cytoplasm to the nucleus. This translocation is controlled by the phosphorylation and degradation of an inhibitor protein called IκB, which forms a complex with NFκB, and thereby holds it in the cytoplasm. Stimulation of the cell by appropriate signals leads to modification of IκB, which in turn results in its dissociation from NFκB.

Binding of the IκB protein to NFκB masks the nuclear localization signal (NLS) of NFκB. Upon stimulation of the cell with specific agents, which depend on the cell type and stage of cell development, IκB is modified in a way that disables binding to NFκB, leading to dissociation of NFκB from IκB. Signals leading to this modification are believed to involve the generation of oxygen radicals, or kinase activation, and to lead to phosphorylation of IκB at specific sites; particularly at Ser-32, Ser-36, and Tyr-42. As a result, its nuclear localization signal is unmasked and NFκB is translocated to the nucleus, where it binds to specific DNA sequences in the regions which control gene expression. NFκB binding to these sites leads to transcription of genes involved in the inflammatory process.

The transcription factor NFκB was originally isolated from mature B cells where it binds to a decameric sequence motif in the κ light chain enhancer. Although NFκB was initially believed to be specific for this cell type and this stage of cell development, NFκB-like proteins have since been identified in a large number of cell types and have been shown to be more generally involved in the induction of gene transcription. This has been further supported by the identification of functionally active NFκB binding sites in several inducible genes.

NFκB is a heterodimeric protein consisting of a 50 kD subunit (p50) and a 65 kD subunit (p65). The cDNAs for p50 and p65 have been cloned and have been shown to be homologous over a region of 300 amino acids. The p50 subunit shows significant homology to the products of the c-rel protooncogene isolated from mammals and birds, and to the Drosophila gene product of dorsal. Recently an additional member of the NFκB family, relB, has been cloned as an immediate early response gene from serum-stimulated fibroblasts.

Both p50 and p65 are capable of forming homodimers, although with different properties: whereas p50 homodimers have strong DNA binding affinity but cannot transactivate transcription, the p65 homodimers can only weakly bind to DNA but are capable of transactivation. p50 is synthesized as the amino-terminal part of the 110 kD precursor (p1110), which has no DNA binding and dimerization activity. The carboxy-terminal part contains eight ankyrin repeats, a motif found in several proteins involved in cell cycle control and differentiation. Cloning of a shorter (2.6 kb) RNA species which is induced in parallel with the 4 kb p50 precursor RNA has revealed that, either by alternative splicing or by differential promoter usage, the C-terminal part of the 110 kD protein can also be expressed independently.

Five IκB family members have been identified: IκB-α, IκB-β, p105/IκB-γ, p100/IκB-Δ, and IκB-ε (Baeuerle and Baltimore, Cell 87:13-20,1996). All IκB-like family members contain multiple ankyrin repeats, which are essential for inhibition of NFκB activation.

The IκB-α-like proteins contain five ankyrin repeats. RL/IF-1 has been cloned and shown to be expressed in regenerating liver within 30 minutes after hepatectomy. Deletion mutagenesis studies have revealed that four out of the five ankyrin repeats of pp40 are essential to inhibit DNA binding activity and to associate with c-rel, and that also the C-terminal region is required. Studies with monospecific antibodies, conducted with the 110 kD p50 precursor, have demonstrated that the C-terminal part (the part with IκB activity) masks the nuclear localization signal (NLS) located in the amino-terminal region of p50. Brown et al. in Science 267:1485-1488 (1995) reported an IκB deletion mutant, lacking 54 NH₂-terminal amino acids, which was neither proteolyzed nor phosphorylated by signals and continued to fully inhibit NFκB. Scheinman et al. and Auphan et al. have reported that glucocorticoid induced immunosuppression is mediated through induction of IκB synthesis (Science, 270:283-285 and 286-290 (1995)).

E2F

According to the invention, PRMT-2 inhibits E2F1 transcriptional activity. E2F transcription activity has an important role in the regulation of cell growth, specifically during the G1/S phase transition. The relevance of E2F transcription factors in the regulation of cell proliferation is underscored by the observation that over-expression of E2F-1 in transgenic mice predisposes them to tumorigenesis. Pierce, et al. (1998) Oncogene 16:1267-76. In cell culture experiments, E2F-1 acts as a potent oncogene in transformation assays. Johnson, et al. (1994) Proc. Natl. Acad. Sci. USA 91:12823-7; Singh, et al. (1994) EMBO J. 13:3329-38. Furthermore, ectopic expression of E2F-1 is sufficient to drive quiescent cells into cell cycle. Johnson, et al. (1993) Nature 365:349-52.

As illustrated herein, PRMT-2 associates with retinoblastoma protein (RB) and requires RB to inhibit E2F1 transcriptional activity. RB is an important regulator of E2F activity. In particular, RB family members whose function is regulated by the G1 cyclin-dependent kinases (cdks) appear to play a role in controlling the activity of the E2F family members. Disruption of various components of this control pathway is a common event during the development of human cancer.

According to the invention, RB also interacts with a protein arginine methyltransferase family member, PRMT-2. PRMT-2 directly interacts with RB through its Ado-Met binding domain, whereas other PRMT-2 proteins, PRMT1, PRMT3, and PRMT4 do not bind RB. As illustrated herein, PRMT-2 and RB interact endogenously. In reporter assays, PRMT-2 repressed E2F1 transcriptional activity in an RB-dependent manner. PRMT-2 formed a ternary complex with E2F1 in the presence of RB. To further explore the role of endogenous PRMT-2 in the regulation of E2F activity, the PRMT-2 gene was ablated in mice by gene targeting. Compared with PRMT-2^{+/+} mouse embryonic fibroblasts (MEFs), the activity of endogenous E2F was endogenously increased in PRMT-2^{-/-} MEFs. Moreover, PRMT-2^{-/-} MEFs exhibited earlier S phase entry following release of serum starvation. Taken together, these findings demonstrate that PRMT-2 can modulate (e.g. inhibit) E2F activity.

Hence, the invention contemplates methods of modulating E2F activity by contacting E2F with a PRMT-2 polypeptide. The invention also contemplates methods of modulating entry of a cell into the cell cycle by contacting the cell with a PRMT-2 polypeptide. The invention further contemplates treating or preventing cancer in an animal by administering to the animal an effective amount of a PRMT-2 polypeptide. For example, in some embodiments, the PRMT-2 polypeptides administered can include any polypeptide with SEQ ID NO:2, 3, 6, or a combination thereof.

Hence, the methods of the invention can be used as proapoptotic, anti-apoptotic, anti-cell cycle progressive, anti-invasive, and anti-metastatic methods. More specifically, the methods of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myclogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

STAT3

As illustrated herein, PRMT2 binds directly to STAT3 (signal transducers and activators of transcription-3) and methylated arginine31 residue of Stat3 through the AdoMet domain of PRMT2, both in vivo and in vitro. Absence of PRMT2 resulted in decreased methylation and a prolonged tyrosine phosphorylation of Stat3. Moreover, PRMT2^{-/-} mice showed significant reductions in weight gain and in food intake. Expression of hypothalamic proopiomelanocortin was significantly increased in leptin-treated PRMT2^{-/-} mice in comparison with leptin treated wild-type controls. These results show that PRMT2 has a pivotal role in weight control through modulation of leptin-Stat3-melanocortin signaling. Thus, PRMT2 is a new target in the treatment of several metabolic disorders, such as food-dependent obesity, hyperlipidemia and type2 diabetes mellitus.

The STAT (signal transducers and activators of transcription) family of proteins are DNA-binding proteins that play a dual role in signal transduction and activation of transcription. Presently, there are six distinct members of the STAT family (STAT1, STAT2, STAT3, STAT4, STAT5, and STAT6) and several isoforms (STAT1α, STAT1β, STAT3α and STAT3β). The activities of the STAT proteins are modulated by various cytokines and mitogenic stimuli. Binding of a cytokine to its receptor results in the activation of Janus protein tyrosine kinases (JAKs) associated with these receptors. This in turn, phosphorylates STAT, resulting in translocation to the nucleus and transcriptional activation of STAT responsive genes. Phosphorylation on a specific tyrosine residue on the STATs results in their activation, resulting in the formation of homodimers and/or heterodimers of STAT, which bind to specific gene promoter sequences. Events mediated by cytokines through STAT activation include cellular proliferation and differentiation, and prevention of apoptosis.

STAT3 (also acute phase response factor (APRF)), in particular, has been found to be responsive to interleukin-6 (IL-6) as well as epidermal growth factor (EGF) (Darnell, Jr., J. E., et al., Science, 1994, 264, 1415-1421). In addition, STAT3 has been found to have an important role in signal transduction by interferons (Yang, C.-H., et al., Proc. Natl. Acad. Sci. USA, 1998, 95, 5568-5572).

As illustrated herein, PRMT-2 can methylate STAT3. Methylation of STAT3 is needed for de-phosphorylation (deactivation) of STAT3. Therefore, according to the invention, PRMT-2 can be used to inhibit the activity of STAT3. For example, in some embodiments, PRMT-2 activity or expression is increased to inhibit the activity of STAT3.

STAT3 is expressed in most cell types (Zhong, Z., et al., Proc. Natl. Acad. Sci. USA, 1994, 91, 4806-4810). It induces the expression of genes involved in response to tissue injury and inflammation. STAT3 has also been shown to prevent apoptosis through the expression of bcl-2 (Fukada, T., et al., Immunity, 1996, 5, 449-460).

Aberrant expression of or constitutive expression of STAT3 is associated with a number of disease processes. For example, STAT3 has been shown to be involved in cell transformation. It is constitutively activated in v-src-transformed cells (Yu, C.-L., et al., Science, 1995, 269, 81-83). Constitutively active STAT3 also induces STAT3 mediated gene expression and is required for cell transformation by src (Turkson, J., et al., Mol. Cell. Biol., 1998, 18, 2545-2552). STAT3 is also constitutively active in Human T cell lymphotropic virus I (HTLV-I) transformed cells (Migone, T.-S. et al., Science, 1995, 269, 79-83). Deactivating STAT3 by increasing PRMT-2 activity or expression can therefore be used to reduce the incidence of cell transformation.

Constitutive activation and/or overexpression of STAT3 appears to be involved in several forms of cancer, including myeloma, breast carcinomas, brain tumors, and leukemias and lymphomas. STAT3 was found to be constitutively active in myeloma tumor cells (Catlett-Falcone, R., et al., Immunity, 1999, 10, 105-115). These cells are resistant to Fas-mediated apoptosis and express high levels of Bcl-xL. Breast cancer cell lines that overexpress EGFR constitutively express phosphorylated STAT3 (Sartor, C. I., et al., Cancer Res., 1997, 57, 978-987; Garcia, R., et al., Cell Growth and Differentiation, 1997, 8, 1267-1276). Activated STAT3 levels were also found to be elevated in low grade glioblastomas and medulloblastomas (Cattaneo, E., et al., Anticancer Res., 1998, 18, 2381-2387). Deactivating STAT3 by increasing PRMT-2 activity or expression can therefore be used to treat myeloma, breast carcinomas, brain tumors, leukemias, lymphomas, glioblastomas and medulloblastomas.

STAT3 has also been found to be constitutively activated in some acute leukemias (Gouilleux-Gruart, V., et al., Leuk. Lymphoma, 1997, 28, 83-88) and T cell lymphoma (Yu, C.-L., et al., J. Immunol., 1997, 159, 5206-5210). Interestingly, STAT3 has been found to be constitutively phosphorylated on a serine residue in chronic lymphocytic leukemia (Frank, D. A., et al., J. Clin. Invest., 1997, 100, 3140-3148). Deactivating STAT3 by increasing PRMT-2 activity or expression can therefore be used to treat acute and chronic leukemias.

STAT3 may also play a role in inflammatory diseases including rheumatoid arthritis. Activated STAT3 has been found in the synovial fluid of rheumatoid arthritis patients (Sengupta, T. K., et al., J. Exp. Med., 1995, 181, 1015-1025) and cells from inflamed joints (Wang, F., et al., J. Exp. Med., 1995, 182, 1825-1831). Deactivating STAT3 by increasing PRMT-2 activity can therefore be used to reduce inflammation and control rheumatoid arthritis.

When STAT3 is methylated it can give rise to an insulin resistant phenotype like that observed in type 2 diabetes. However, as provided by the invention, inhibition of STAT3 methylation gives rise to an insulin sensitive phenotype. In particular, PRMT-2 knockout mice had increased insulin sensitivity, gained less weight and had reduced food intake compared to wild type mice on a similar diet (mouse chow). Serum concentrations of fasting glucose, triglycerides, free fatty acids and insulin in PRMT-2 knockout mice were lower than those of wild type mice. Histological analysis revealed that glycogen content was decreased in the liver of PRMT-2 knockout mice. Glucose and insulin tolerance tests showed that PRMT-2 knockout mice had more rapid clearance of glucose and greater responsiveness to insulin compared to wild type mice. Tyrosine phosphorylation of insulin receptor substrate-1 (IRS-1) was enhanced in skeletal muscle from insulin-treated PRMT-2 knockout mice. Taken together, these data indicate that inhibition of PRMT-2 activity or expression can modulate glucose and lipid metabolism, and help control body weight. PRMT-2 may therefore be a new target in the treatment of several metabolic disorders, such as type 2 diabetes mellitus, food dependent obesity and hyperlipidemia.

Hence, the invention also provides a method for reducing methylation of STAT3 by inhibiting the activity or expression of PRMT-2. The invention also provides a method for treating obesity, diabetes, hyperlipidemia and insulin-related disorders in a mammal by administering to the mammal an effective amount of an agent that can inhibit PRMT-2 activity or expression.

Modulating PRMT-2 Activity or Expression

According to the invention, any agents that modulate the activity or expression of PRMT-2 can be utilized in the invention. Such agents can act directly or indirectly on the PRMT-2 gene or the PRMT-2 gene product. Such agents can act at the transcriptional, translational or protein level to modulate the activity or expression of PRMT-2. The term “modulate” or “modulating” means changing, that is increasing or decreasing. Hence, while agents that can decrease PRMT-2 expression or PRMT-2 activity can be used in the compositions and methods of the invention, agents that also increase PRMT-2 expression or activity are also encompassed within the scope of the invention. Moreover, PRMT-2 polypeptides and nucleic acids can be used as agents that increase PRMT-2 expression or activity. For example, a nucleic acid or expression cassette that includes SEQ ID NO:1 can be administered to promote expression of PRMT-2. Similarly, a PRMT-2 polypeptide can be administered to increase the activity of PRMT-2 in a cell or a mammal. Examples of PRMT-2 polypeptides include those with SEQ ID NO:2, 3, 4 or 6. Generally, PRMT-2 polypeptides with SEQ ID NO:2, 3 or 6 are preferably administered when increased PRMT-2 activity is desired.

In other embodiments, one of skill in the art may choose to decrease PRMT-2 expression, translation or activity. For example, the degradation of PRMT-2 mRNA may be increased upon exposure to small duplexes of synthetic double-stranded RNA through the use of RNA interference (siRNA or RNAi) technology (Scherr, M. et al. 2003; Martinez, L. A. et al. 2002). A process is therefore provided for inhibiting expression of a PRMT-2 gene in a cell. The process comprises introduction of RNA with partial or fully double-stranded character into the cell or into the extracellular environment. Inhibition is specific to PRMT-2 RNA because a nucleotide sequence from a portion of the PRMT-2 gene is chosen to produce inhibitory RNA. This process is effective in producing inhibition of PRMT-2 gene expression.

SiRNAs can be designed using the guidelines provided by Ambion (Austin, Tex.). Briefly, the PRMT-2 cDNA sequence (e.g. SEQ ID NO:1) is scanned for target sequences that have AA dinucleotides. Sense and anti-sense oligonucleotides can be generated to these targets that contain a G/C content, for example, of about 35 to 55%. These sequences can then be compared to others in the human genome database to minimize homology to other known coding sequences (e.g. by performing a Blast search using the information available through the NCBI database). siRNAs designed in this manner can be used to modulate PRMT-2 expression.

Mixtures and combinations of such siRNA molecules are also contemplated by the invention. These compositions can be used in the methods of the invention, for example, for treating or preventing obesity, diabetes, hyperlipidemia, excessive weight gain, insulin-related disorders as well as other conditions that are typically responsive to inhibition of NFκB or that are responsive to methylated STAT3. These compositions are also useful for modulating (e.g. decreasing) PRMT-2 expression, or for modulating NFκB or STAT3 activity.

The siRNA provided herein can selectively hybridize to RNA in vivo or in vitro. A nucleic acid sequence is considered to be “selectively hybridizable” to a reference nucleic acid sequence if the two sequences specifically hybridize to one another under physiological conditions or under moderate stringency hybridization and wash conditions. In some embodiments the siRNA is selectively hybridizable to an RNA (e.g. a PRMT-2 RNA) under physiological conditions. Hybridization under physiological conditions can be measured as a practical matter by observing interference with the function of the RNA. Alternatively, hybridization under physiological conditions can be detected in vitro by testing for siRNA hybridization using the temperature (e.g. 37° C.) and salt conditions that exist in vivo.

Moreover, as an initial matter, other in vitro hybridization conditions can be utilized to characterize siRNA interactions. Exemplary in vitro conditions include hybridization conducted as described in the Bio-Rad Labs ZetaProbe manual (Bio-Rad Labs, Hercules, Calif.); Sambrook et al., Molecular Cloning: A Laboratory Manual, 2nd ed., Cold Spring Harbor Laboratory Press, (1989), or Sambrook et al., Molecular Cloning: A Laboratory Manual, 3rd ed., Cold Spring Harbor Laboratory Press, (2001)), expressly incorporated by reference herein.

For example, hybridization can be conducted in 1 mM EDTA, 0.25 M Na₂ HPO₄ and 7% SDS at 42° C., followed by washing at 42° C. in 1 mM EDTA, 40 mM NaPO₄, 5% SDS, and 1 mM EDTA, 40 mM NaPO₄, 1% SDS. Hybridization can also be conducted in 1 mM EDTA, 0.25 M Na₂HPO₄ and 7% SDS at 60° C., followed by washing in 1 mM EDTA, 40 mM NaPO₄, 5% SDS, and 1 mM EDTA, 40 mM NaPO₄, 1% SDS. Washing can also be conducted at other temperatures, including temperatures ranging from 37° C. to at 65° C., from 42° C. to at 65° C., from 37° C. to at 60° C., from 50° C. to at 65° C., from 37° C. to at 55° C., and other such temperatures.

The siRNA employed in the compositions and methods of the invention may be synthesized either in vivo or in vitro. In some embodiments, the siRNA molecules are synthesized in vitro using methods, reagents and synthesizer equipment available to one of skill in the art. Endogenous RNA polymerases within a cell may mediate transcription in vivo, or cloned RNA polymerase can be used for transcription in vivo or in vitro. For transcription from a transgene or an expression construct in vivo, a regulatory region may be used to transcribe the siRNA strands.

Depending on the particular sequence utilized and the dose of double stranded siRNA material delivered, the compositions and methods may provide partial or complete loss of function for the target gene (PRMT-2). A reduction or loss of gene expression in at least 99% of targeted cells has been shown for other genes. See, e.g., U.S. Pat. No. 6,506,559. Lower doses of injected material and longer times after administration of the selected siRNA may result in inhibition in a smaller fraction of cells.

The siRNA may comprise one or more strands of polymerized ribonucleotide; it may include modifications to either the phosphate-sugar backbone or the nucleoside. The double-stranded siRNA structure may be formed by a single self-complementary RNA strand or two complementary RNA strands. siRNA duplex formation may be initiated either inside or outside the cell. The siRNA may be introduced in an amount that allows delivery of at least one copy per cell. Higher doses of double-stranded material may yield more effective inhibition.

Inhibition is sequence-specific in that nucleotide sequences corresponding to the duplex region of the RNA are targeted for genetic inhibition. siRNA containing nucleotide sequences identical to a portion of the target gene is preferred for inhibition. However, siRNA sequences with insertions, deletions, and single point mutations relative to the target sequence have also been found to be effective for inhibition. Thus, sequence identity may optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

The siRNA may be directly introduced into the cell (i.e., intracellularly); or introduced extracellularly into a cavity, interstitial space, into the circulation of an organism, introduced orally, or may be introduced by bathing an organism in a solution containing siRNA. Methods for oral introduction include direct mixing of siRNA with food of the organism, as well as engineered approaches in which a species that is used as food is engineered to express an siRNA, then fed to the organism to be affected. Physical methods of introducing nucleic acids include injection directly into the cell or extracellular injection into the organism of an siRNA solution.

The siRNA may also be delivered in vitro to cultured cells using transfection agents available in the art such as lipofectamine or by employing viral delivery vectors such as those from lentiviruses. Such in vitro delivery can be performed for testing purposes or for therapeutic purposes. For example, cells from a patient can be treated in vitro and then re-administered to the patient.

The advantages of using siRNA include: the ease of introducing double-stranded siRNA into cells, the low concentration of siRNA that can be used, the stability of double-stranded siRNA, and the effectiveness of the inhibition. The ability to use a low concentration of a naturally-occurring nucleic acid avoids several disadvantages of anti-sense interference.

Anti-sense nucleic acids can also be used to inhibit the function of PRMT-2. In general, the function of PRMT-2 RNA is inhibited, for example, by administering to a mammal a nucleic acid that can inhibit the functioning of PRMT-2 RNA. Nucleic acids that can inhibit the function of a PRMT-2RNA can be generated from coding and non-coding regions of the PRMT-2 gene. However, nucleic acids that can inhibit the function of a PRMT-2 RNA are often selected to be complementary to PRMT-2 nucleic acids that are naturally expressed in the mammalian cell to be treated with the methods of the invention. In some embodiments, the nucleic acids that can inhibit PRMT-2 RNA functions are complementary to PRMT-2 sequences found near the 5′ end of the PRMT-2 coding region. For example, nucleic acids that can inhibit the function of a PRMT-2 RNA can be complementary to the 5′ region of SEQ ID NO:1.

A nucleic acid that can inhibit the functioning of a PRMT-2 RNA need not be 100% complementary to SEQ ID NO:1. Instead, some variability in the sequence of the nucleic acid that can inhibit the functioning of a PRMT-2 RNA is permitted. For example, a nucleic acid that can inhibit the functioning of a PRMT-2 RNA from a human can be complementary to a nucleic acid encoding either a human or another mammalian PRMT-2 gene product.

Moreover, nucleic acids that can hybridize under moderately or highly stringent hybridization conditions to a nucleic acid comprising SEQ ID NO:1 are sufficiently complementary to inhibit the functioning of a PRMT-2 RNA and can be utilized in the methods of the invention.

“Stringent hybridization conditions” and “stringent hybridization wash conditions” in the context of nucleic acid hybridization are somewhat sequence dependent, and may differ depending upon the environmental conditions of the nucleic acid. For example, longer sequences tend to hybridize specifically at higher temperatures. An extensive guide to the hybridization of nucleic acids is found in Tijssen, Laboratory Techniques in Biochemistry and Molecular biology-Hybridization with Nucleic Acid Probes, page 1, chapter 2 “Overview of principles of hybridization and the strategy of nucleic acid probe assays” Elsevier, New York (1993). See also, J. Sambrook et al., Molecular Cloning: A Laboratory Manual, Cold Spring Harbor Press, N.Y., pp 9.31-9.58 (1989); J. Sambrook et al., Molecular Cloning: A Laboratory Manual, Cold Spring Harbor Press, N.Y. (3rd ed. 2001).

Generally, highly stringent hybridization and wash conditions are selected to be about 5° C. lower than the thermal melting point (Tₘ) for the specific double-stranded sequence at a defined ionic strength and pH. The Tₘ is the temperature (under defined ionic strength and pH) at which 50% of a complementary target sequence hybridizes to a perfectly matched probe. For example, under “highly stringent conditions” or “highly stringent hybridization conditions” a nucleic acid will hybridize to its complement to a detectably greater degree than to other sequences (e.g., at least 2-fold over background). By controlling the stringency of the hybridization and/or washing conditions nucleic acids that are 100% complementary can be hybridized.

For DNA-DNA hybrids, the Tₘ can be approximated from the equation of Meinkoth and Wahl Anal. Biochem. 138:267-284 (1984):
Tₘ=81.5° C.+16.6(log M)+0.41 (% GC)−0.61 (% form)−500/L
where M is the molarity of monovalent cations, % GC is the percentage of guanosine and cytosine nucleotides in the DNA, % form is the percentage of formamide in the hybridization solution, and L is the length of the hybrid in base pairs.

Very stringent conditions are selected to be equal to the Tₘ for a particular probe. Alternatively, stringency conditions can be adjusted to allow some mismatching in sequences so that lower degrees of similarity can hybridize. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl and 0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

The degree of complementarity or sequence identity of hybrids obtained during hybridization is typically a function of post-hybridization washes, the critical factors being the ionic strength and temperature of the final wash solution. The type and length of hybridizing nucleic acids also affects whether hybridization will occur and whether any hybrids formed will be stable under a given set of hybridization and wash conditions.

An example of stringent hybridization conditions for hybridization of complementary nucleic acids that have more than 100 complementary residues on a filter in a Southern or Northern blot is 50% formamide with 1 mg of heparin at 42° C., with the hybridization being carried out overnight. An example of highly stringent conditions is 0.15 M NaCl at 72° C. for about 15 minutes. An example of stringent wash conditions is a 0.2×SSC wash at 65° C. for 15 minutes (see also, Sambrook, infra). Often, a high stringency wash is preceded by a low stringency wash to remove background probe signal. An example of medium stringency for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C.

Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to hybridize to homologous nucleic acids that are substantially identical to reference nucleic acids of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

In general, Tₘ is reduced by about 1° C. for each 1% of mismatching. Thus, Tₘ, hybridization, and/or wash conditions can be adjusted to hybridize to sequences of the desired sequence identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ).

If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part 1, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.). Using these references and the teachings herein on the relationship between Tₘ, mismatch, and hybridization and wash conditions, those of ordinary skill can generate variants of the present homocysteine S-methyltransferase nucleic acids.

Precise complementarity is therefore not required for successful duplex formation between a nucleic acid that can inhibit a PRMT-2 RNA and the complementary coding sequence of a PRMT-2 RNA. Inhibitory nucleic acid molecules that comprise, for example, 2, 3, 4, or 5 or more stretches of contiguous nucleotides that are precisely complementary to a PRMT-2 coding sequence, each separated by a stretch of contiguous nucleotides that are not complementary to adjacent PRMT-2 coding sequences, can inhibit the function of PRMT-2 RNA. In general, each stretch of contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an anti-sense nucleic acid hybridized to a sense nucleic acid to determine the degree of mismatching that will be tolerated between a particular anti-sense nucleic acid and a particular PRMT-2 RNA.

Nucleic acids that are complementary a PRMT-2 RNA can be administered to a mammal or to directly to the site where the PRMT-2 activity is to be inhibited. Alternatively, nucleic acids that are complementary to a PRMT-2 RNA can be generated by transcription from an expression cassette that has been administered to a mammal. For example, a complementary RNA can be transcribed from a PRMT-2 nucleic acid that has been inserted into an expression cassette in the 3′ to 5′ orientation, that is, opposite to the usual orientation employed to generate sense RNA transcripts. Hence, to generate a complementary RNA that can inhibit the function of an endogenous PRMT-2 RNA, the promoter would be positioned to transcribe from a 3′ site towards the 5′ end of the PRMT-2 coding region.

In some embodiments an RNA that can inhibit the function of an endogenous PRMT-2 RNA is an anti-sense oligonucleotide. The anti-sense oligonucleotide is complementary to at least a portion of the coding sequence of a gene comprising SEQ ID NO:1. Such anti-sense oligonucleotides are generally at least six nucleotides in length, but can be about 8, 12, 15, 20, 25, 30, 35, 40, 45, or 50 nucleotides long. Longer oligonucleotides can also be used.

Anti-sense oligonucleotides can be composed of deoxyribonucleotides, ribonucleotides, or a combination of both. Oligonucleotides can be synthesized endogenously from transgenic expression cassettes or vectors as described herein. Alternatively, such oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, 1994, Meth. Mol. Biol. 20:1-8; Sonveaux, 1994, Meth. Mol. Biol. 26:1-72; Uhlmann et al., 1990, Chem. Rev. 90:543-583.

PRMT-2 anti-sense oligonucleotides can be modified without affecting their ability to hybridize to a PRMT-2 RNA. These modifications can be internal or at one or both ends of the anti-sense molecule. For example, internucleoside phosphate linkages can be modified by adding peptidyl, cholesteryl or diamine moieties with varying numbers of carbon residues between these moieties and the terminal ribose. Modified bases and/or sugars, such as arabinose instead of ribose, or a 3′,5′-substituted oligonucleotide in which the 3′ hydroxyl group or the 5′ phosphate group are substituted, can also be employed in a modified anti-sense oligonucleotide. These modified oligonucleotides can be prepared by methods available in the art. Agrawal et al., 1992, Trends Biotechnol. 10:152-158; Uhlmann et al., 1990, Chem. Rev. 90:543-584; Uhlmann et al., 1987, Tetrahedron. Lett. 215:3539-3542.

In one embodiment of the invention, expression of a PRMT-2 gene is decreased using a ribozyme. A ribozyme is an RNA molecule with catalytic activity. See, e.g., Cech, 1987, Science 236: 1532-1539; Cech, 1990, Ann. Rev. Biochem. 59:543-568; Cech, 1992, Curr. Opin. Struct. Biol. 2: 605-609; Couture and Stinchcomb, 1996, Trends Genet. 12: 510-515. Ribozymes can be used to inhibit gene function by cleaving an RNA sequence, as is known in the art (see, e.g., Haseloff et al., U.S. Pat. No. 5,641,673).

PRMT-2 nucleic acids complementary to SEQ ID NO:1 can be used to generate ribozymes that will specifically bind to mRNA transcribed from a PRMT-2 gene. Methods of designing and constructing ribozymes that can cleave other RNA molecules in trans in a highly sequence specific manner have been developed and described in the art (see Haseloff et al. (1988), Nature 334:585-591). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201). The target sequence can be a segment of about 10, 12, 15, 20, or 50 contiguous nucleotides selected from a nucleotide sequence shown in SEQ ID NO:1. Longer complementary sequences can be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target.

Screening for Agents that Modulate PRMT-2 Activity or Expression

The invention also provides a method for identifying a test agent that can modulate Protein Arginine N-Methyltransferase-2 expression in a cell comprising contacting the cell with a test agent and observing whether expression of a nucleic acid comprising SEQ ID NO:1 is modulated relative to expression of a nucleic acid comprising SEQ ID NO:1 in a cell that was not contacted with the test agent.

Further methods are also provided for identifying a test agent that can modulate Protein Arginine N-Methyltransferase-2 activity in a test cell comprising contacting the test cell with a test agent and observing whether Protein Arginine N-Methyltransferase-2 activity is modulated relative to Protein Arginine N-Methyltransferase-2 activity in a control cell that was not contacted with the test agent.

Any cell type or test agent available to one skill in the art can be employed. In some embodiments the cell can be an embryonic cell, a cancer cell or an immune cell. In other embodiments, the cell can be a cultured cell that has been exposed to an interleukin or a cytokine to induce the cell to respond as though it were having an inflammatory response.

Antibodies

According to the invention antibodies raised against PRMT-2 can also be used to modulate PRMT-2 activity. In some embodiments, such antibodies inhibit PRMT-2 activity. In other embodiments, anti-PRMT-2 antibodies can be used to activate or mimic PRMT-2 activity.

Thus, the invention also contemplates antibodies that can bind to a PRMT-2 polypeptide of the invention. In another embodiment, a disease involving insulin insensitivity, hyperlipidemia, obesity or one where STAT-3 activity or expression is undesirably active can be treated by administering to a mammal an antibody that can bind to PRMT-2 polypeptide. For example, the antibody can be directed against a PRMT-2 polypeptide comprising any one of SEQ ID NO:2, SEQ ID NO:3, SEQ ID NO:4, SEQ ID NO:6, or a combination thereof.

All antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82, 4592-4596 (1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector function, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody that includes the variable domain complementarity determining regions (CDR), and the like forms, all of which fall under the broad term “antibody,” as used herein. The present invention contemplates the use of any specificity of an antibody, polyclonal or monoclonal, and is not limited to antibodies that recognize and immunoreact with a specific epitope. In some embodiments, however, the antibodies of the invention may react with selected epitopes within the Ado-Met or other domains of the PRMT-2 protein.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments, which are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments retain some ability to selectively bind with its antigen or receptor and are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.

(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad Sci. USA 90: 6444-6448 (1993).

The preparation of polyclonal antibodies is well-known to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

The preparation of monoclonal antibodies likewise is conventional. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Methods of in vitro and in vivo manipulation of monoclonal antibodies are also available to those skilled in the art. For example, the monoclonal antibodies to be used in accordance with the present invention may be made by the hybridoma method first described by Kohler and Milstein, Nature 256, 495 (1975), or they may be made by recombinant methods, for example, as described in U.S. Pat. No. 4,816,567. The monoclonal antibodies for use with the present invention may also be isolated from antibody libraries using the techniques described in Clackson et al. Nature 352: 624-628 (1991), as well as in Marks et al., J. Mol Biol. 222: 581-597 (1991).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Another method for generating antibodies involves a Selected Lymphocyte Antibody Method (SLAM). The SLAM technology permits the generation, isolation and manipulation of monoclonal antibodies without the process of hybridoma generation. The methodology principally involves the growth of antibody forming cells, the physical selection of specifically selected antibody forming cells, the isolation of the genes encoding the antibody and the subsequent cloning and expression of those genes.

More specifically, an animal is immunized with a source of specific antigen. The animal can be a rabbit, mouse, rat, or any other convenient animal. This immunization may consist of purified protein, in either native or recombinant form, peptides, DNA encoding the protein of interest or cells expressing the protein of interest. After a suitable period, during which antibodies can be detected in the serum of the animal (usually weeks to months), blood, spleen or other tissues are harvested from the animal. Lymphocytes are isolated from the blood and cultured under specific conditions to generate antibody-forming cells, with antibody being secreted into the culture medium. These cells are detected by any of several means (complement mediated lysis of antigen-bearing cells, fluorescence detection or other) and then isolated using micromanipulation technology. The individual antibody forming cells are then processed for eventual single cell PCR to obtain the expressed Heavy and Light chain genes that encode the specific antibody. Once obtained and sequenced, these genes are cloned into an appropriate expression vector and recombinant, monoclonal antibody produced in a heterologous cell system. These antibodies are then purified via standard methodologies such as the use of protein A affinity columns. These types of methods are further described in Babcook, et al., Proc. Natl. Acad. Sci. (USA) 93: 7843-7848 (1996); U.S. Pat. No. 5,627,052; and PCT WO 92/02551 by Schrader.

Another method involves humanizing a monoclonal antibody by recombinant means to generate antibodies containing human specific and recognizable sequences. See, for review, Holmes, et al., J. Immunol., 158:2192-2201 (1997) and Vaswani, et al., Annals Allergy, Asthma & Immunol., 81:105-115 (1998). The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the antibody is obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad Sci. 81, 6851-6855 (1984).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab monovalent fragments. Alternatively, an enzymatic cleavage using pepsin produces two monovalent Fab′ fragments and an Fc fragment directly. These methods are described, for example, in U.S. Pat. No. 4,036,945 and No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science 242:423-426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention further contemplates human and humanized forms of non-human (e.g. murine) antibodies. Such humanized antibodies can be chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies can comprise substantially all of at least one, and typically two, variable domains, in which all or substantially all of the CDR regions correspond to those of a non-human immunoglobulin and all or substantially all of the Fv regions are those of a human immunoglobulin consensus sequence. The humanized antibody optimally also will comprise at least a portion of an immunoglobulin constant region (Fc), typically that of a human immunoglobulin. For further details, see: Jones et al., Nature 321, 522-525 (1986); Reichmann et al., Nature 332, 323-329 (1988); Presta, Curr. Op. Struct. Biol. 2, 593-596 (1992); Holmes, et al., J. Immunol., 158:2192-2201 (1997) and Vaswani, et al., Annals Allergy, Asthma & Immunol., 81:105-115 (1998); U.S. Pat. Nos. 4,816,567 and 6,331,415; PCT/GB84/00094; PCT/US86/02269; PCT/US89/00077; PCT/US88/02514; and WO91/09967, each of which is incorporated herein by reference in its entirety.

The invention also provides methods of mutating antibodies to optimize their affinity, selectivity, binding strength or other desirable property. A mutant antibody refers to an amino acid sequence variant of an antibody. In general, one or more of the amino acid residues in the mutant antibody is different from what is present in the reference antibody. Such mutant antibodies necessarily have less than 100% sequence identity or similarity with the reference amino acid sequence. In general, mutant antibodies have at least 75% amino acid sequence identity or similarity with the amino acid sequence of either the heavy or light chain variable domain of the reference antibody. Preferably, mutant antibodies have at least 80%, more preferably at least 85%, even more preferably at least 90%, and most preferably at least 95% amino acid sequence identity or similarity with the amino acid sequence of either the heavy or light chain variable domain of the reference antibody.

The antibodies of the invention are isolated antibodies. An isolated antibody is one that has been identified and separated and/or recovered from a component of the environment in which it was produced. Contaminant components of its production environment are materials that would interfere with diagnostic or therapeutic uses for the antibody, and may include enzymes, hormones, and other proteinaceous or nonproteinaceous solutes. The term “isolated antibody” also includes antibodies within recombinant cells because at least one component of the antibody's natural environment will not be present. Ordinarily, however, isolated antibody will be prepared by at least one purification step.

If desired, the antibodies of the invention can be purified by any available procedure. For example, the antibodies can be affinity purified by binding an antibody preparation to a solid support to which the antigen used to raise the antibodies is bound. After washing off contaminants, the antibody can be eluted by known procedures. Those of skill in the art will know of various techniques common in the immunology arts for purification and/or concentration of polyclonal antibodies, as well as monoclonal antibodies (see for example, Coligan, et al., Unit 9, Current Protocols in Immunology, Wiley Interscience, 1991, incorporated by reference).

In some embodiments, the antibody will be purified as measurable by at least three different methods: 1) to greater than 95% by weight of antibody as determined by the Lowry method, and most preferably more than 99% by weight; 2) to a degree sufficient to obtain at least 15 residues of N-terminal or internal amino acid sequence by use of a spinning cup sequenator; or 3) to homogeneity by SDS-PAGE under reducing or non-reducing conditions using Coomassie blue or, preferably, silver stain.

In some embodiments, the antibody or fragment thereof may be conjugated to a therapeutic moiety such as a cytotoxin, e.g., a cytostatic or cytocidal agent, a therapeutic agent or a radioactive metal ion. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Expression of PRMT-2 Nucleic Acids

Mammalian expression of PRMT-2 sense, anti-sense, ribozyme, and siRNA nucleic acids can be accomplished as described in Dijkema et al., EMBO J. (1985) 4: 761, Gorman et al., Proc. Natl. Acad. Sci. USA (1982b) 79: 6777, Boshart et al., Cell (1985) 41: 521 and U.S. Pat. No. 4,399,216. Other features of mammalian expression can be facilitated as described in Ham and Wallace, Meth. Enz. (1979) 58: 44, Barnes and Sato, Anal. Biochem. (1980) 102: 255, U.S. Pat. Nos. 4,767,704, 4,657,866, 4,927,762, 4,560,655, WO 90/103430, WO 87/00195, and U.S. Pat. No. RE 30,985.

PRMT-2 nucleic acids can be placed within linear or circular molecules. They can be placed within autonomously replicating molecules or within molecules without replication sequences. They can be regulated by their own or by other regulatory sequences, as is known in the art.

PRMT-2 nucleic acids can be used in expression cassettes or gene delivery vehicles, for the purpose of delivering an mRNA or oligonucleotide (with a sequence from a native mRNA or its complement), a full-length protein, a fusion protein, a polypeptide, a ribozyme, a siRNA or a single-chain antibody, into a cell, preferably a eukaryotic cell. According to the present invention, a gene delivery vehicle can be, for example, naked plasmid DNA, a viral expression vector comprising a sense or anti-sense nucleic acid of the invention, or a sense or anti-sense nucleic acid of the invention in conjunction with a liposome or a condensing agent.

PRMT-2 nucleic acids can be introduced into suitable host cells using a variety of techniques that are available in the art, such as transferrin-polycation-mediated DNA transfer, transfection with naked or encapsulated nucleic acids, liposome-mediated DNA transfer, intracellular transportation of DNA-coated latex beads, protoplast fusion, viral infection, electroporation and calcium phosphate-mediated transfection.

In one embodiment of the invention, the gene delivery vehicle comprises a promoter and one of the PRMT-2 nucleic acids disclosed herein. Preferred promoters are tissue-specific promoters and promoters that are activated by cellular proliferation, such as the thymidine kinase and thymidylate synthase promoters. Other preferred promoters include promoters that are activated by infection with a virus, such as the α- and β-interferon promoters, and promoters that can be activated by a hormone, such as estrogen. Other promoters that can be used include the Moloney virus LTR, the CMV promoter, and the mouse albumin promoter.

A gene delivery vehicle can comprise viral sequences such as a viral origin of replication or packaging signal. These viral sequences can be selected from viruses such as astrovirus, coronavirus, orthomyxovirus, papovavirus, paramyxovirus, parvovirus, picomavirus, poxvirus, retrovirus, togavirus or adenovirus. In some embodiments, the gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91102805).

Examples of retroviruses that can be utilized include avian leukosis virus (ATCC Nos. VR-535 and VR-247), bovine leukemia virus (VR-1315), murine leukemia virus (MLV), mink-cell focus-inducing virus (Koch et al., J. Vir. 49:828, 1984; and Oliff et al., J. Vir. 48:542, 1983), murine sarcoma virus (ATCC Nos. VR-844, 45010 and 45016), reticuloendotheliosis virus (ATCC Nos. VR-994, VR-770 and 45011), Rous sarcoma virus, Mason-Pfizer monkey virus, baboon endogenous virus, endogenous feline retrovirus (e.g., RD114), and mouse or rat gL30 sequences used as a retroviral vector. Strains of MLV from which recombinant retroviruses can be generated include 4070A and 1504A (Hartley and Rowe, J. Vir. 19:19, 1976), Abelson (ATCC No. VR-999), Friend (ATCC No. VR-245), Graffi (Ru et al., J. Vir. 67:4722, 1993; and Yantchev Neopksma 26:397, 1979), Gross (ATCC No. VR-590), Kirsten (Albino et al., J. Exp. Med. 164:1710, 1986), Harvey sarcoma virus (Manly et al., J. Vir. 62:3540, 1988; and Albino et al., J. Exp. Med. 164:1710, 1986) and Rauscher (ATCC No. VR-998), and Moloney MLV (ATCC No. VR-190). A non-mouse retrovirus that can be used is Rous sarcoma virus, for example, Bratislava (Manly et al., J. Vir. 62:3540, 1988; and Albino et al., J. Exp. Med. 164:1710, 1986), Bryan high titer (e.g., ATCC Nos. VR-334, VR-657, VR-726, VR-659, and VR-728), Bryan standard (ATCC No. VR-140), Carr-Zilber (Adgighitov et al., Neoplasma 27:159, 1980), Engelbreth-Holm (Laurent et al., Biochem Biophys Acta 908:241, 1987), Harris, Prague (e.g., ATCC Nos. VR-772, and 45033), or Schmidt-Ruppin (e.g. ATCC Nos. VR-724, VR-725, VR-354) viruses.

Any of the above retroviruses can be readily utilized in order to assemble or construct retroviral gene delivery vehicles given the disclosure provided herein and standard recombinant techniques (e.g., Sambrook et al., Molecular Cloning: A Laboratory Manual, 2^{nd} Edition (1989), Sambrook et al., Molecular Cloning: A Laboratory Manual, 3^{rd} Edition (2001), and Kunkle, Proc. Natl. Acad. Sci. U.S.A. 82:488, 1985). Portions of retroviral expression vectors can be derived from different retroviruses. For example, retrovector LTRs can be derived from a murine sarcoma virus, a tRNA binding site from a Rous sarcoma virus, a packaging signal from a murine leukemia virus, and an origin of second strand synthesis from an avian leukosis virus. These recombinant retroviral vectors can be used to generate transduction competent retroviral vector particles by introducing them into appropriate packaging cell lines (see Ser. No. 07/800,921, filed Nov. 29, 1991).

Recombinant retroviruses can be produced that direct the site-specific integration of the recombinant retroviral genome into specific regions of the host cell DNA. Such site-specific integration is useful for mutating the endogenous PRMT-2 gene. Site-specific integration can be mediated by a chimeric integrase incorporated into the retroviral particle (see Ser. No. 08/445,466 filed May 22, 1995). It is preferable that the recombinant viral gene delivery vehicle is a replication-defective recombinant virus.

Packaging cell lines suitable for use with the above-described retroviral gene delivery vehicles can be readily prepared (see WO 92/05266) and used to create producer cell lines (also termed vector cell lines or “VCLs”) for production of recombinant viral particles. In preferred embodiments of the present invention, packaging cell lines are made from human (e.g., HT1080 cells) or mink parent cell lines, thereby allowing production of recombinant retroviral gene delivery vehicles that are capable of surviving inactivation in human serum. The construction of recombinant retroviral gene delivery vehicles is described in detail in WO 91/02805. These recombinant retroviral gene delivery vehicles can be used to generate transduction competent retroviral particles by introducing them into appropriate packaging cell lines. Similarly, adenovirus gene delivery vehicles can also be readily prepared and utilized given the disclosure provided herein (see also Berkner, Biotechniques 6:616-627, 1988, and Rosenfeld et al., Science 252:431-434, 1991, WO 93/07283, WO 93/06223, and WO 93/07282).

A gene delivery vehicle can also be a recombinant adenoviral gene delivery vehicle. Such vehicles can be readily prepared and utilized given the disclosure provided herein (see also Berkner, Biotechniques 6:616, 1988, and Rosenfeld et al., Science 252:431, 1991, WO 93/07283, WO 93/06223, and WO 93/07282). Adeno-associated viral gene delivery vehicles can also be constructed and used to deliver proteins or nucleic acids of the invention to cells in vitro or in vivo. The use of adeno-associated viral gene delivery vehicles in vitro is described in Chatteijee et al., Science 258: 1485-1488 (1992), Walsh et al., Proc. Nat'l. Acad. Sci. 89: 7257-7261 (1992), Walsh et al., J. Clin. Invest. 94: 1440-1448 (1994), Flotte et al., J. Biol. Chem. 268: 3781-3790 (1993), Ponnazhagan et al., J. Exp. Med. 179: 733-738 (1994), Miller et al., Proc. Nat'l Acad. Sci. 91: 10183-10187 (1994), Einerhand et al., Gene Ther. 2: 336-343 (1995), Luo et al., Exp. Hematol. 23: 1261-1267 (1995), and Zhou et al., Gene Therapy 3: 223-229 (1996). In vivo use of these vehicles is described in Flotte et al., Proc. Nat'l Acad. Sci. 90: 10613-10617(1993), and Kaplitt et al., Nature Genet. 8:148-153 (1994).

In another embodiment of the invention, a gene delivery vehicle is derived from a togavirus. Such togaviruses include alphaviruses such as those described in U.S. Ser. No. 08/405,627, filed Mar. 15, 1995, WO 95/07994. Alpha viruses, including Sindbis and ELVS viruses can be gene delivery vehicles for nucleic acids of the invention. Alpha viruses are described in WO 94/21792, WO 92/10578 and WO 95/07994. Several different alphavirus gene delivery vehicle systems can be constructed and used to deliver nucleic acids to a cell according to the present invention. Representative examples of such systems include those described in U.S. Pat. Nos. 5,091,309 and 5,217,879. Preferred alphavirus gene delivery vehicles for use in the present invention include those that are described in WO 95/07994.

The recombinant viral vehicle can also be a recombinant alphavirus viral vehicle based on a Sindbis virus. Sindbis constructs, as well as numerous similar constructs, can be readily prepared. Sindbis viral gene delivery vehicles typically comprise a 5′ sequence capable of initiating Sindbis virus transcription, a nucleotide sequence encoding Sindbis non-structural proteins, a viral junction region inactivated so as to prevent fragment transcription, and a Sindbis RNA polymerase recognition sequence. Optionally, the viral junction region can be modified so that nucleic acid transcription is reduced, increased, or maintained. As will be appreciated by those in the art, corresponding regions from other alphaviruses can be used in place of those described above.

The viral junction region of an alphavirus-derived gene delivery vehicle can comprise a first viral junction region that has been inactivated in order to prevent transcription of the nucleic acid and a second viral junction region that has been modified such that nucleic acid transcription is reduced. An alphavirus-derived vehicle can also include a 5′ promoter capable of initiating synthesis of viral RNA from cDNA and a 3′ sequence that controls transcription termination.

Other recombinant togaviral gene delivery vehicles that can be utilized in the present invention include those derived from Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309 and 5,217,879 and in WO 92/10578.

Other viral gene delivery vehicles suitable for use in the present invention include, for example, those derived from poliovirus (Evans et al., Nature 339:385, 1989, and Sabin et al., J. Biol. Standardization 1:115, 1973) (ATCC VR-58); rhinovirus (Arnold et al., J. Cell. Biochem. L401, 1990) (ATCC VR-1110); pox viruses, such as canary pox virus or vaccinia virus (Fisher-Hoch et al., PROC. NATL. ACAD. SCI. U.S.A. 86:317, 1989; Flexner et al., Ann. N.Y. Acad. Sci. 569:86, 1989; Flexner et al., Vaccine 8:17, 1990; U.S. Pat. Nos. 4,603,112 and 4,769,330; WO 89/01973) (ATCC VR-111; ATCC VR-2010); SV40 (Mulligan et al., Nature 277:108, 1979) (ATCC VR-305), (Madzak et al., J. Gen. Vir. 73:1533, 1992); influenza virus (Luytjes et al., Cell 59:1107, 1989; McMicheal et al., The New England Journal of Medicine 309:13, 1983; and Yap et al., Nature 273:238, 1978) (ATCC VR-797); parvovirus such as adeno-associated virus (Samulski et al., J. Vir. 63:3822, 1989, and Mendelson et al., Virology 166:154, 1988) (ATCC VR-645); herpes simplex virus (Kit et al., Adv. Exp. Med. Biol. 215:219, 1989) (ATCC VR-977; ATCC VR-260); Nature 277: 108, 1979); human immunodeficiency virus (EPO 386,882, Buchschacher et al., J. Vir. 66:2731, 1992); measles virus (EPO 440,219) (ATCC VR-24); A (ATCC VR-67; ATCC VR-1247), Aura (ATCC VR-368), Bebaru virus (ATCC VR-600; ATCC VR-1240), Cabassou (ATCC VR-922), Chikungunya virus (ATCC VR-64; ATCC VR-1241), Fort Morgan (ATCC VR-924), Getah virus (ATCC VR-369; ATCC VR-1243), Kyzylagach (ATCC VR-927), Mayaro (ATCC VR-66), Mucambo virus (ATCC VR-580; ATCC VR-1244), Ndumu (ATCC VR-371), Pixuna virus (ATCC VR-372; ATCC VR-1245), Tonate (ATCC VR-925), Triniti (ATCC VR-469), Una (ATCC VR-374), Whataroa (ATCC VR-926), Y-62-33 (ATCC VR-375), O'Nyong virus, Eastern encephalitis virus (ATCC VR-65; ATCC VR-1242), Western encephalitis virus (ATCC VR-70; ATCC VR-1251; ATCC VR-622; ATCC VR-1252), and coronavirus (Hamre et al., Proc. Soc. Exp. Biol. Med. 121:190, 1966) (ATCC VR-740).

A nucleic acid of the invention can also be combined with a condensing agent to form a gene delivery vehicle. In a preferred embodiment, the condensing agent is a polycation, such as polylysine, polyarginine, polyornithine, protamine, spermine, spermidine, and putrescine. Many suitable methods for making such linkages are known in the art (see, for example, Ser. No. 08/366,787, filed Dec. 30, 1994).

In an alternative embodiment, a nucleic acid is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell that has internalized the liposome, thereby releasing its contents into the cytoplasm. Prior to interaction with the surface of a cell, however, the liposome membrane acts as a relatively impermeable barrier that sequesters and protects its contents, for example, from degradative enzymes. Additionally, because a liposome is a synthetic structure, specially designed liposomes can be produced that incorporate desirable features. See Stryer, Biochemistry, pp. 236-240, 1975 (W. H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., Proc. Natl. Acad. Sci. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising nucleic acids such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al, J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin™, from GIBCO BRL, Grand Island, N.Y. See also Feigner et al., Proc. Natl. Acad. Sci. US491: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE) and the like. These materials can also be mixed with the DOTMA and DOTAP starting materials in appropriate ratios. Methods for making liposomes using these materials are well known in the art.

The liposomes can comprise multilamellar vesicles (MLVs), small unilamellar vesicles (SUVs), or large unilamellar vesicles (LUVs). The various liposome-nucleic acid complexes are prepared using methods known in the art. See, e.g., Straubinger et al., Methods of Immunology (1983), Vol. 101, pp. 512-527; Szoka et al., Proc. Natl. Acad. Sci. USA 87:3410-3414, 1990; Papahadjopoulos et al., Biochim. Biophys. Acta 394:483, 1975; Wilson et al., Cell 17:77, 1979; Deamer and Bangham, Biochim. Biophys. Acta 443:629, 1976; Ostro et al., Biochem. Biophys. Res. Commun. 76:836, 1977; Fraley et al., Proc. Natl. Acad Sci. USA 76:3348, 1979; Enoch and Strittmatter, Proc. Natl. Acad Sci. USA 76:145, 1979; Fraley et al., J. Biol. Chem. 255:10431, 1980; Szoka and Papahadjopoulos, Proc. Natl. Acad. Sci. USA 75:145, 1979; and Schaefer-Ridder et al., Science 215:166, 1982.

In addition, lipoproteins can be included with a nucleic acid of the invention for delivery to a cell. Examples of such lipoproteins include chylomicrons, HDL, IDL, LDL, and VLDL. Mutants, fragments, or fusions of these proteins can also be used. Modifications of naturally occurring lipoproteins can also be used, such as acetylated LDL. These lipoproteins can target the delivery of nucleic acids to cells expressing lipoprotein receptors. Preferably, if lipoproteins are included with a nucleic acid, no other targeting ligand is included in the composition.

Receptor-mediated targeted delivery of BAFF/TNFsf13b nucleic acids to specific tissues can also be used. Receptor-mediated DNA delivery techniques are described in, for example, Findeis et al. (1993), Trends in Biotechnol. 11, 202-05; Chiou et al. (1994), GENE THERAPEUTICS: METHODS AND APPLICATIONS OF DIRECT GENE TRANSFER (J. A. Wolff, ed.); Wu & Wu (1988), J. Biol. Chem. 263, 621-24; Wu et al. (1994), J. Biol. Chem. 269, 542-46; Zenke et al. (1990), Proc. Natl. Acad. Sci. U.S.A. 87, 3655-59; Wu et al. (1991), J. Biol. Chem. 266, 338-42.

In another embodiment, naked nucleic acid molecules are used as gene delivery vehicles, as described in WO 90/11092 and U.S. Pat. No. 5,580,859. Such gene delivery vehicles can be either DNA or RNA and, in certain embodiments, are linked to killed adenovirus. Curiel et al., Hum. Gene. Ther. 3:147-154, 1992. Other suitable vehicles include DNA-ligand (Wu et al., J. Biol. Chem. 264:16985-16987, 1989), lipid-DNA combinations (Feigner et al., Proc. Natl. Acad. Sci. USA 84:7413 7417, 1989), liposomes (Wang et al., Proc. Natl. Acad Sci. 84:7851-7855, 1987) and microprojectiles (Williams et al., Proc. Natl. Acad. Sci. 88:2726-2730, 1991).

One can increase the efficiency of naked nucleic acid uptake into cells by coating the nucleic acids onto biodegradable latex beads. This approach takes advantage of the observation that latex beads, when incubated with cells in culture, are efficiently transported and concentrated in the perinuclear region of the cells. The beads will then be transported into cells when injected into muscle. Nucleic acid-coated latex beads will be efficiently transported into cells after endocytosis is initiated by the latex beads and thus increase gene transfer and expression efficiency. This method can be improved further by treating the beads to increase their hydrophobicity, thereby facilitating the disruption of the endosome and release of nucleic acids into the cytoplasm.

PRMT-2-specific siRNA, ribozymes and anti-sense nucleic acids can be introduced into cells in a similar manner. The nucleic acid construct encoding the siRNA, ribozyme or anti-sense nucleic acid may include transcriptional regulatory elements, such as a promoter element, an enhancer or UAS element, and a transcriptional terminator signal, for controlling transcription of the ribozyme in the cells. Mechanical methods, such as microinjection, liposome-mediated transfection, electroporation, or calcium phosphate precipitation, can be used to introduce the siRNA, ribozyme or anti-sense DNA construct into cells whose division it is desired to decrease, as described above. Alternatively, if it is desired that the cells stably retain the DNA construct, the DNA construct can be supplied on a plasmid and maintained as a separate element or integrated into the genome of the cells, as is known in the art.

Expression of an endogenous PRMT-2 gene in a cell can also be altered by introducing in frame with the endogenous PRMT-2 gene a DNA construct comprising a PRMT-2 targeting sequence, a regulatory sequence, an exon, and an unpaired splice donor site by homologous recombination, such that a homologous recombinant cell comprising the DNA construct is formed. The new transcription unit can be used to turn the PRMT-2 gene on or off as desired. This method of affecting endogenous gene expression is taught in U.S. Pat. No. 5,641,670.

Integration of a delivered PRMT-2 nucleic acid into the genome of a cell line or tissue can be monitored by any means known in the art. For example, Southern blotting of the delivered PRMT-2 nucleic acid can be performed. A change in the size of the fragments of a delivered nucleic acid indicates integration. Replication of a delivered nucleic acid can be monitored inter alia by detecting incorporation of labeled nucleotides combined with hybridization to a PRMT-2 probe. Expression of a PRMT-2 nucleic acid can be monitored by detecting production of PRMT-2 mRNA that hybridizes to the delivered nucleic acid or by detecting PRMT-2 protein. PRMT-2 protein can be detected immunologically.

Compositions

The PRMT-2 polypeptides and antibodies of the invention, including their salts, as well as the PRMT-2 siRNA, ribozymes, sense and anti-sense nucleic acids are administered to modulate PRMT-2 expression or activity, or to achieve a reduction in at least one symptom associated with a condition, indication, infection or disease associated with inappropriate NFκB activity, E2F1 transcriptional activity or STAT3 activity. Other agents can be included such as other NFκB, E2F1 or STAT3 antagonists, cytotoxins active against a variety of cell types, cytokines and the like.

In some embodiments the therapeutic agent of the invention are administered in a “therapeutically effective amount.” Such a therapeutically effective amount is used herein to identify an amount sufficient to obtain the desired physiological effect, e.g., treatment of a condition, disorder, disease and the like or reduction in symptoms of the condition, disorder, disease and the like.

To achieve the desired effect(s), the PRMT-2 polypeptide, nucleic acid, antibody, and combinations with other agents thereof, may be administered as single or divided dosages. For example, PRMT-2 polypeptides and antibodies can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the polypeptide or antibody chosen, the disease, the weight, the physical condition, the health, the age of the mammal, whether prevention or treatment is to be achieved, and if the polypeptide or antibody is chemically modified. Such factors can be readily determined by the clinician employing animal models or other test systems that are available in the art.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the therapeutic agents and compositions of the invention may be essentially continuous over a preselected period of time or may be in a series of spaced doses. Both local and systemic administration is contemplated.

To prepare the composition, polypeptides, nucleic acids, antibodies cytokines, cytotoxins and other agents are synthesized or otherwise obtained, purified as necessary or desired and then lyophilized and stabilized. These therapeutic agents can then be adjusted to the appropriate concentration, and optionally combined with other agents. The absolute weight of a given polypeptide, nucleic acid, antibody cytokine or cytotoxin included in a unit dose can vary widely. For example, about 0.01 to about 2 g, or about 0.1 to about 500 mg, of at least one polypeptide, nucleic acid, antibody, cytokine, or cytotoxin of the invention, or a plurality of polypeptides, nucleic acids, antibodies, cytokines or cytotoxins can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the therapeutic agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

Thus, one or more suitable unit dosage forms comprising the therapeutic agents of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic agents may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are generally combined with a pharmaceutically acceptable carrier, diluent or excipient to form a pharmaceutical formulation, or unit dosage form. For oral administration, the therapeutic agents may be present as a powder, a granular formulation, a solution, a suspension, an emulsion or in a natural or synthetic polymer or resin for ingestion of the active ingredients from a chewing gum. The therapeutic agents may also be presented as a bolus, electuary or paste. Orally administered therapeutic agents of the invention can also be formulated for sustained release, e.g., the therapeutic agents can be coated, micro-encapsulated, or otherwise placed within a sustained delivery device. The total active ingredients in such formulations comprise from 0.1 to 99.9% by weight of the formulation.

By “pharmaceutically acceptable” it is meant a carrier, diluent, excipient, and/or salt that is compatible with the other ingredients of the formulation, and not deleterious to the recipient thereof.

Pharmaceutical formulations containing the therapeutic agents of the invention can be prepared by procedures known in the art using well-known and readily available ingredients. For example, the therapeutic agents can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the therapeutic agents of the invention can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one therapeutic agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more therapeutic agents of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic agents of the invention can also take the form of an aqueous or anhydrous solution or dispersion, or alternatively the form of an emulsion or suspension or salve.

Thus, the therapeutic agents may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The active polypeptides, nucleic acids or antibodies and other ingredients may form suspensions, solutions, or emulsions in oily or aqueous vehicles, and may contain formulatory agents such as suspending, stabilizing and/or dispersing agents. Alternatively, the active polypeptides, nucleic acids or antibodies and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if necessary, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

Additionally, the polypeptides or antibodies are well suited to formulation as sustained release dosage forms and the like. The formulations can be so constituted that they release the therapeutic agents, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

For topical administration, the therapeutic agents may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic agents of the invention can be delivered via patches or bandages for dermal administration. Alternatively, the polypeptide or antibody can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The therapeutic agents can also be delivered via iontophoresis, e.g., as disclosed in U.S. Pat. No. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic agents in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agents may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The therapeutic agents of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific infection, indication or disease. Any statistically significant attenuation of one or more symptoms of an infection, indication or disease that has been treated pursuant to the method of the present invention is considered to be a treatment of such infection, indication or disease within the scope of the invention.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

Therapeutic agents of the present invention can also be administered in an aqueous solution when administered in an aerosol or inhaled form. Thus, other aerosol pharmaceutical formulations may comprise, for example, a physiologically acceptable buffered saline solution containing between about 0.1 mg/ml and about 100 mg/ml of one or more of the therapeutic agents of the present invention specific for the indication or disease to be treated. Dry aerosol in the form of finely divided solid polypeptide, nucleic acid or antibody particles that are not dissolved or suspended in a liquid are also useful in the practice of the present invention. Polypeptides, nucleic acids or antibodies of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic agents of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

Furthermore, the active ingredients may also be used in combination with other therapeutic agents, for example, pain relievers, anti-inflammatory agents, antihistamines, anti-cancer agents, anti-obesity agents, anti-viral agents (e.g. an anti-HIV agent), antimicrobial agents, bronchodilators and the like, whether for the conditions described or some other condition.

The present invention further pertains to a packaged pharmaceutical composition for modulating PRMT-2 expression or activity such as a kit or other container. The kit or container holds a therapeutically effective amount of a pharmaceutical composition for modulating PRMT-2 activity or expression and instructions for using the pharmaceutical composition for modulating PRMT-2 activity or expression. The pharmaceutical composition includes at least one PRMT-2 polypeptide, siRNA, ribozyme, anti-sense nucleic acid or antibody of the present invention, in a therapeutically effective amount such that PRMT-2 activity or expression is modulated. The composition can also contain an anti-inflammatory agent, an anti-cancer agent, and anti-viral agent (e.g. anti-HIV agent), an anti-obesity agent, an appetite suppressant or similar agent.

The invention will be further described by reference to the following detailed examples, which are given for illustration of the invention, and are not intended to be limiting thereof.

### EXAMPLE 1

### PRMT-2 Inhibits NF-κB Function and Promotes Apoptosis

The protein arginine methyltransferases (PRMTs) include a family of proteins with related putative methyltransferase domains that modify chromatin and regulate cellular transcription. Although some family members, PRMT1 and PRMT4, have been implicated in transcriptional modulation or intracellular signaling. Chen, D. et al. Science 284, 2174-2177 (1999); Koh et al. J. Biol. Chem. 276, 1089-1098 (2001); Mowen, K. A. et al. Cell 104, 731-741 (2001); Wang, H. et al. Science 293, 853-857 (2001); Xu, W. et al. Science 294, 2507-2511 (2001). However, the roles of PRMTs, including PRMT-2, in diverse cellular processes have not been fully established.

This example illustrates that PRMT-2 inhibits NF-κB-dependent transcription. PRMT-2 exerted this effect by causing nuclear accumulation of IκBα, which is concomitantly decreased nuclear NF-κB DNA binding. Mutation or deletion of the highly conserved S-adenosyl methionine binding domain of PRMT-2 abolished its ability to inhibit κB-dependent transcription. PRMT-2 also rendered cells susceptible to apoptosis by cytokines or cytotoxic drugs, possibly due to its effects on NF-κB. Embryo fibroblasts from PRMT-2 genetic knockout strains of mice had increased NF-κB activity and decreased susceptibility to apoptosis compared to wild type cells. These results implicate PRMT-2 in the regulation of cell activation and programmed cell death.

### Materials and Methods

Plasmids. HIV-1-CAT (wt and mutant), HIV-2-CAT, HTLV-1-CAT, HTLV-2-CAT and HIV-1-luciferase (Luc) reporter systems, both wild type and mutant, are described in Nabel, G. & Baltimore, D. Nature 326, 711-713 (1987); Leung, K. & Nabel, G. J. Nature 333, 776-778 (1988); Markovitz, D. M. et al. Proc. Natl. Acad. Sci. USA 87, 9098-9102 (1990). The Rous sarcoma virus (RSV) expression plasmids containing the p50 and p65 cDNAs were also employed. Duckett, C. S. et al. Mol. Cell. Biol. 13, 1315-1322 (1993). The human PRMT1, PRMT-2, and PRMT3 cDNAs were cloned by RT-PCR using total RNA extracted from Jurkat cells. PRMT-2-A is an alternative splice variant of PRMT-2. Katsanis et al. Mammalian Genome 8, 526-529 (1997). PRMT-2-A was cloned by PCR using a human B cell library as template.

The following primers pair were used for PCR: 5′-AAGTCGACGCCATGGCAACATCAGGTGACTGT-3′ (SEQ ID NO:8) and 5′-AAGCGGCCGCTT ATCTCCAGATGGGGAAGACTT-3′ (SEQ ID NO:9) for human PRMT-2; 5′-AAGGATCCGCGAACTGCAT CATGGAGAA-3′ (SEQ ID NO:10) and 5′-AAAAGCTTAAACCGCCTAGGAACGCTCA-3′ (SEQ ID NO:11) for human PRMT1; 5′-AAGATATCGCCATGG ACGAGCCAGAACTGTCGGACAGCGGGGACGAGGCCGCCTGG GAGGATGAGGACGAT-3′ (SEQ ID NO:12) and 5′-AATCTAGATT ACTGGAGACCATAAGTTTGAGTTG-3′ (SEQ ID NO:13) for human PRMT3; 5′-AAGTCGACGCCATGGCAACATCAGGTGACTGT-3′ (SEQ ID NO:14) and 5′-AATCTAGATTAAAATGAATCACGCACGACCCTT-3′ (SEQ ID NO:15) for PRMT-2-A.

All these cDNA coding regions were subcloned into the pVR1012 mammalian expression vector (Danthinne et al. J. Virol. 72, 9201-9207 (1998)) with HA-tag at the C-terminus.

The four-alanine mutant of PRMT-2 (PRMT-2-4A) was generated from the wild type pVR1012 PRMT-2 construct using the Stratagene Quickchange™ Site-Directed Mutagenesis kit, according to the manufacturer's directions. The sequence of the sense mutagenic oligonucleotide used is: 5′-ATAAAGAATCCCTG ACGGATAAAG CCGCAGCCGCGGTGGGCTGTGGGACTGGGATCATC-3′ (SEQ ID NO:16). This mutation introduced a unique SalI site within the PRMT-2 sequence. Mutant clones were identified by restriction of the isolated plasmid DNA with SalI, and verified by sequencing.

The PRMT-2-N (PRMT-21-95 amino acids) mutant was generated from the wild type pVR1012 PRMT-2 construct by PCR using the primer pairs: 5′-GCGCGCGATATCGCCATGGCAACATCAGGTGACTGT-3′ (SEQ ID NO:17) and 5′-GCGCGCTCTAGACTAGGCATAGTCAGGCACGTCA TAAGGATA GGGGTCGTACTCATCCACGT-3′ (SEQ ID NO:18). Wild type PRMT-2-A was subcloned into pGEX-6P (Amersham Pharmacia) for generation of glutathione-S-transferase (GST)-fusion proteins. The luciferase reporter, 2×kB-Luc, was a gift from Dr. Colin Duckett. Another luciferase reporter, 5 kB-Luc, was purchased from Stratagene. The expression vector for the IκBα mutant (S32A/S36A) was described previously. Wu et al. J. Virol. 71, 3161-3167 (1997).

A wild type IKK2 expression plasmid was used as a template to create a constitutively active form by site-directed mutagenesis (Stratagene): 5′-GAGCTGGATCAGGGCGAGCTCTGCACAGAATTCGTGGGGACCCTG-3′ (SEQ ID NO:40) and 5′-CAGGGTCCCCACGAATTCTGTGCAGAG CTCGCCCTGATCCAGCTC-3′ (SEQ ID NO:41) (Suh et al. (2002) Prostate 52: 183-200). The resulting constitutively active IKK2 fragment was amplified by PCR and cloned into an RSV expression vector. RSV expression vector was created by replacing the CMV promoter in the pVR1012 vector with an RSV promoter.

Cell culture, transfection, and reporter gene assays. The E1A-transformed, human kidney cell line, 293, and NIH-3T3 cells were maintained in Dulbecco's Modified Eagle Medium (DMEM) supplemented with 10% fetal calf serum and penicillin-streptomycin at 37° C. in 5% carbon dioxide in tissue culture grade Petri dishes. PRMT-2−/− mouse embryo fibroblasts (MEFs) and wild type MEFs were prepared from day 13.5 embryos and maintained in DMEM supplemented with 10% fetal calf serum. MEFs at passage 4 were used in this experiment. Lipofectamine Plus™ reagent (Boehringer Mannheim) was used to transfect both 293 and NIH-3T3 cells according to directions from the manufacturer. The transfection efficiency of both 293 and NIH-3T3 cells using Lipofectamine Plus™ reagent was found to be constant and reproducible, with standard deviations of ˜10% as assayed by β-Gal assays and FACS analysis of a cotransfected CD2 expression vector. TNF-α stimulation of cells was done using recombinant TNF-α (200 U/ml) for 12 hours. Transfected cells were harvested at 36 hours, and CAT activity was assayed on 10 to 100 μg of protein from whole cell extracts. CAT assays were performed essentially as described in Leung, K. & Nabel, G. J. Nature 333, 776-778 (1988). To analyze the κB-reporter activity in MEFs, cells were transfected with the reporter (2×kB-Luc) and PRL-TK vector (Promega) using FuGENE6 transfection reagent (Roche). Luciferase activity was analyzed by Dual-Luciferase Reporter Assay System (Promega).

DNA binding assay. Electrophoretic mobility shift assays (EMSAs) were conducted on 10 μg of nuclear extract protein from 293 cells transiently transfected with pRSV p50/p65 expression constructs and pVR1012 PRMT-2-A/PRMT-2-N expression constructs. A modified Dignam procedure (Dignam et al. Nucleic Acids Res. 11, 1475-1489 (1983)) was used to prepare nuclear extracts from 293 cells. Perkins, N. D. et al. Science 275, 523-527 (1997). NF-KB DNA binding was assayed using a double-stranded 32P-labeled κB probe (Geneka Biotechnology). DNA binding assays were performed as described previously. Perkins et al. Science 275, 523-527 (1997). Supershifting was done using NF-κB p65 (C-20) and NF-κB p50 (H-119) (Santa Cruz). GST-PRMT-2-A fusion proteins were expressed in BL21 (DE3) cells and extracts were prepared as described previously. Smith, D. B. & Johnson, K. S. Gene 67, 31-40 (1988).

To determine if PRMT-2-A interfered with the dimerization of p50/p65, immunoprecipitations were carried out in IP buffer (20 mM HEPES, 150 mM KCl, 100 mM NaCl, 2.5 mM MgCl₂, 0.5% NP40, 1 mM DTT, protease inhibitor cocktail (Complete™: Boehringer Mannheim)) using α-p65-antibody conjugated beads (p65 A (AC), Santa Cruz) from 293 nuclear extracts that had been transfected with either PRMT-2-A or PRMT-2-N. The complexes were resolved by 4-15% SDS-PAGE and transferred to PVDF. p50 was detected by Western blotting using a p50 antibody (H-119) (Santa Cruz).

Small-scale preparation of nuclear extracts. A modified Dignam procedure (Dignam et al., Nucleic Acids Res. 11: 1475-89 (1983)) was used to prepare nuclear extracts from 293 cells. Cells were harvested, washed with PBS, resuspended in 1 ml Dignam buffer A, and transferred to pre-chilled microfuge tubes, which were spun at 1000 rpm for 1 minute. The supernatant was aspirated thoroughly, and pellets were carefully resuspended to avoid frothing, in 60 μl modified Dignam buffer A containing 0.1% Nonidet P-40 (NP40). Samples were incubated at 4° C. for approximately 10 minutes, and microcentrifuged for 10 minutes at 4° C. The cytoplasmic extract supernatant was diluted in 3 volumes of modified Dignam buffer D and frozen quickly. The pellets were resuspended in 40 μl Dignam buffer C. Samples were incubated for 15 minutes at 4° C. on a tumbler, and centrifuged again for 5 minutes at 4° C. The supernatant was diluted with 6 volumes of modified Dignam buffer D. Samples were frozen quickly in aliquots and stored at −70° C.

Fusion proteins. GST proteins were expressed in BL21 (DE3) cells and extracts were prepared as described by Smith and Johnson (Gene 67: 31-40 (1988)). GST fusion proteins were purified using glutathione-Sepharose beads (Pharmacia) and washed three times with Immunoprecipitation (IP) buffer (20 mM HEPES, 150 mM KCl, 100 mM NaCl, 2.5 mM M9C12, 0.5% NP40, 1 mM DTT, protease inhibitor cocktail (Complete™: Boehringer Mannheim)).

Western Blotting. Proteins resolved by SDS-polyacrylamide gel electrophoresis (PAGE) were transferred to polyvinylidene difluoride (PVDF) membranes. Membranes were blocked with 5% nonfat dry milk (NFDM), 2.5% Bovine Serum Albumin (BSA) in Tris-buffered saline (TBS) containing 0.5% Tween 20 (TBS-Tween) for 10 minutes at room temperature and then incubated with primary antibody in TBS-Tween-milk-BSA for 1-2 hrs at room temperature or overnight at 4° C. Following three 15 minute washes in TBS-Tween, membranes were incubated for 1 hour with the appropriate horseradish peroxidase-conjugated secondary antibody (Santa Cruz) in TBS-Tween-milk-BSA. After two more washes in TBS-Tween and a rinse in phosphate-buffered saline (PBS), the immunoreactive proteins were visualized by enhanced chemiluminescence (Amersham).

In vitro association assay. To determine whether PRMT2 interfered with the dimerization of p50/p65, immunoprecipitations were carried out in IP buffer using α-p65-antibody conjugated beads (p65 A (AC), Santa Cruz) from 293 nuclear extracts that had been transfected with either wild type or mutant. The complexes were resolved by 4-15% SDS-PAGE and transferred to PVDF. p50 was detected by Western blotting using a p50 antibody (H-119) (Santa Cruz). For binding of p65 to p300/TAF_{II}250, immunoprecipitations were carried out using either a p300 NH₂-terminal antibody (NM11, Pharmingen) or the TAF_{II}250antibody (6B3, Santa Cruz) from 293 nuclear extracts that had been transfected with either PRMT2 or PRMT2-N. p65 was detected by Western blotting using a p65 antibody (Santa Cruz).

For binding of GST methyltransferase proteins to NF-κB, pBluescript constructs of p50/p65 were transcribed and translated in vitro using the TNT-T7-coupled reticulocyte lysate system (Promega) with [³⁵S]methionine (Amersham Pharmacia) in accordance with the manufacturer's instructions. A 10 μl volume of the reaction product was incubated in IP buffer with purified GST-methyltransferase proteins. After incubation at 4° C. for 1 hour, the beads were washed three times with IP buffer. The bound proteins were solubilized in sodium dodecyl sulfate (SDS) sample buffer, subjected to SDS-PAGE and visualized by autoradiography.

In vitro methyltransferase reactions. In vitro methyltransferase reactions were carried out as described previously (Chen et al., 1999). Mixed calf thymus histones (Boehringer-Mannheim) were incubated for 30 min at 30° C. in 30 μl reactions containing 20 mM Tris-HCl, 0.2 M NaCl, 4 mM EDTA (pH 8.0); 10 pg mixed histones; PRMT1/PRMT2 immunoprecipitated from 100 μg transfected 293 whole cell extract protein; and 7 μM S-adenosyl-L-[methyl³H]methionine (specific activity of 14.7 Ci/mmol). Reactions were stopped by the addition of SDS-PAGE sample buffer. The reactions were then subjected to SDS-PAGE on 10-20% Tris-HCl gradient gels (BioRad). Gels were stained with Coomassie blue to visualize histone bands and then the incorporated label was enhanced using Enhance (NEN Life Sciences) and subjected to fluorography for 1-5 days at −70° C. on sensitized Kodak Biomax film. Film images were digitized using a scanner equipped with a film scanning unit. Bands were quantified using Imagequant software.

Immunohistochemistry and confocal microscopy. PRMT2^{-/-} fibroblasts were transfected with a HA-tagged PRMT2 expression vector. 36 hrs after transfection the cells were treated with TNF-α for 30 min. The media was then removed and cells were incubated for an additional 30 minutes in the presence or absence of LMB. Cells were fixed, permeabilized with CytoFix-CytoPerm (BD Biosciences) for 20 minutes, and washed with Perm/Wash buffer (BD Biosciences). IκBα (Rabbit Polyclonal antibody, 1:1000; Santa Cruz Biotechnology) and HA (Rat Monoclonal antibody, 1:500; Roche) were diluted in Perm/Wash buffer and incubated for 1 hr. After two washes cells were stained with anti rabbit Alexa 488 and anti rat Alexa 564 (1:1000, Invitrogen) for 30 min. Cells were washed and mounted with Ultracruz mounting media (Santa Cruz) containing DAPI. Confocal microscopy was performed using a Leica confocal microscope.

Apoptosis analysis. Apoptosis in PRMT-2-expressing 293 cells was analyzed as follows. Cells were seeded at 2.5×10⁵ per well in 6 well plates. The next day, empty vector, mutant IκBα (S32A/S36A), RelA or PRMT-2 was co-transfected with CD2 expression vector. Twenty-four hours after transfection cells were stimulated with TNF-α (1000 U/ml) for 24 hours. Both floating and attached cells in each well were harvested by EDTA treatment. Cells were stained with APC-labeled anti-CD2 antibody (BD Bioscience) in SM buffer (PBS containing 2% FCS). After washing twice with PBS, cells were stained with FITC-labeled Annexin V and propidium iodide using Annexin V FITC Apoptosis Detection Kit (Oncogene), and analyzed by flow cytometry (FACS Caliber, BD Bioscience).

Cell viability in PRMT-2^{+/+} and PRMT-2^{-/-} MEFs after etoposide exposure was analyzed as follows. Etoposide is a DNA-damaging agent with pro-apoptotic activity. Cells were seeded at 2.5×10⁵ per well in 6 well plates and 12 hours later were transfected with control PRMT2 expression plasmids. Twenty-four hours after transfection, cells were stimulated with etoposide (0, 50 and 100 μM) for 24 hours. Cells were then treated with trypsin and stained with trypan blue (Invitrogen). Unstained surviving cells were counted with a hemocytometer. The net difference in survival cell number between the untreated group and the etoposide group was treated as dead cells, and cell death rate was calculated as a ratio of the number of dead cells versus the number of untreated cells. Apoptosis caused by etoposide was confirmed by microscopic observation using FITC-annexin V staining according to the manufacturer's instructions (annexin V FITC Apoptosis Detection Kit, Oncogene).

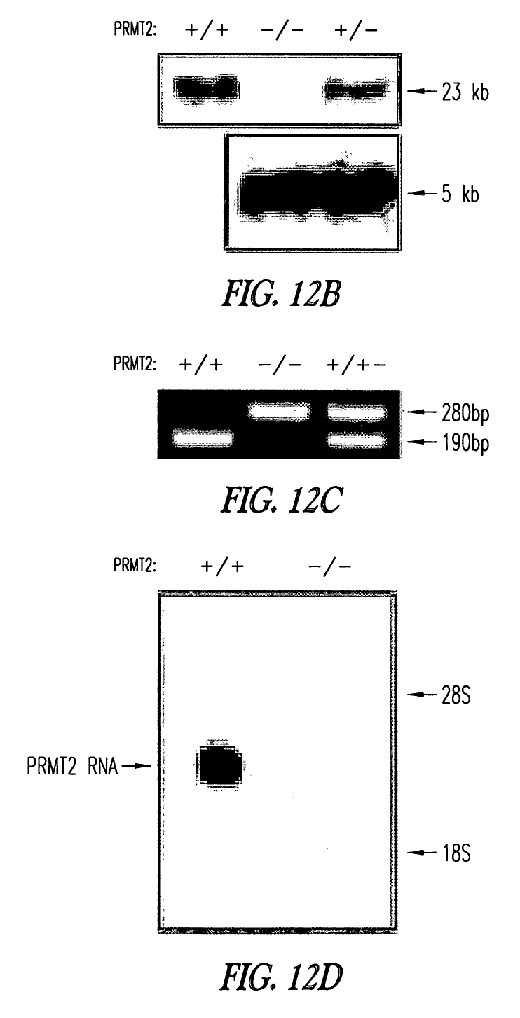

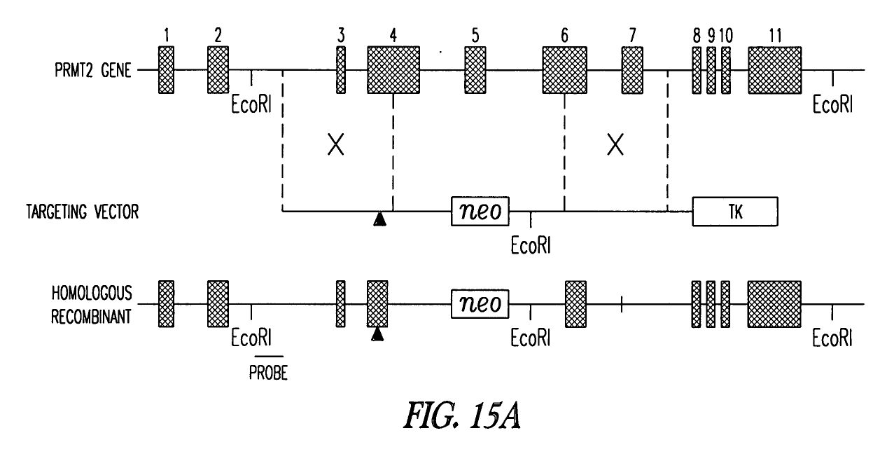

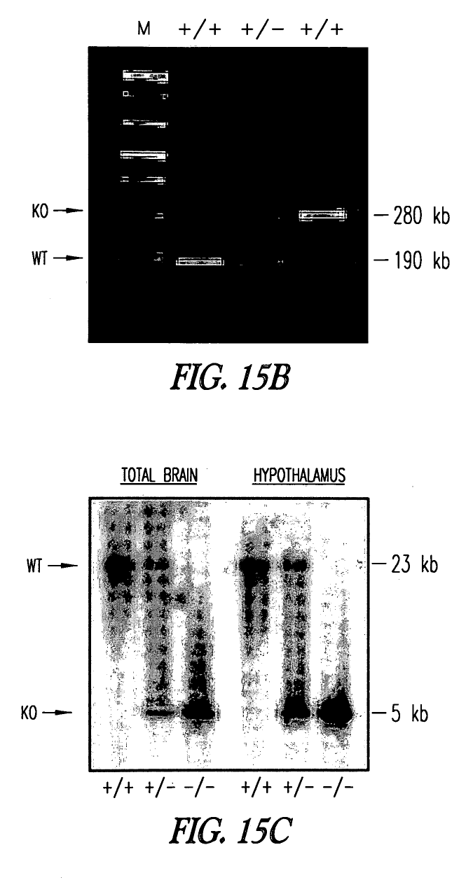

Null PRMT-2 Mice. The generation of null PRMT-2 (PRMT-2^{-/-}) mice was as shown in FIGS. 12 and 15.

### Results

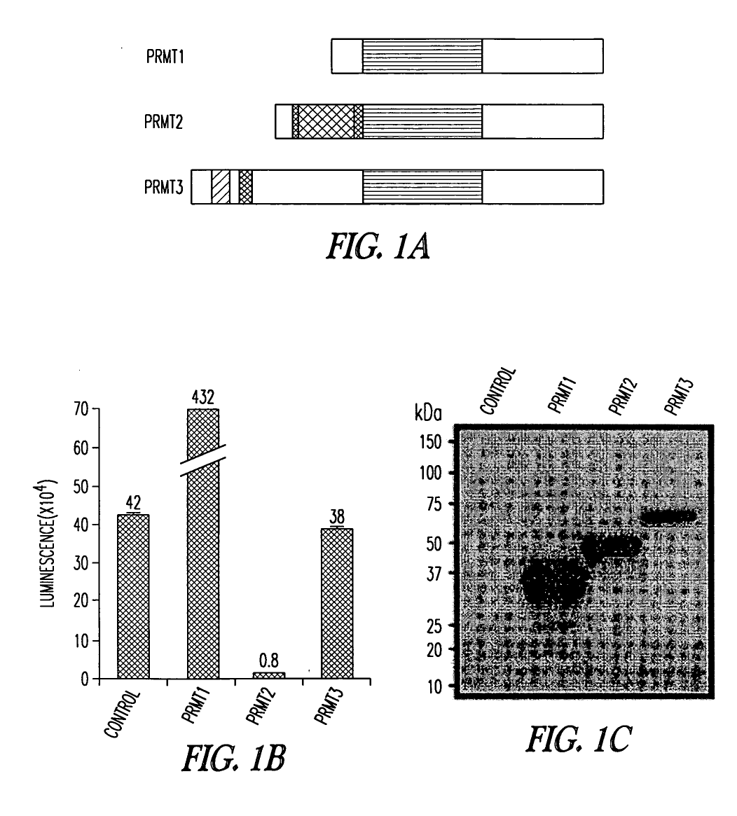

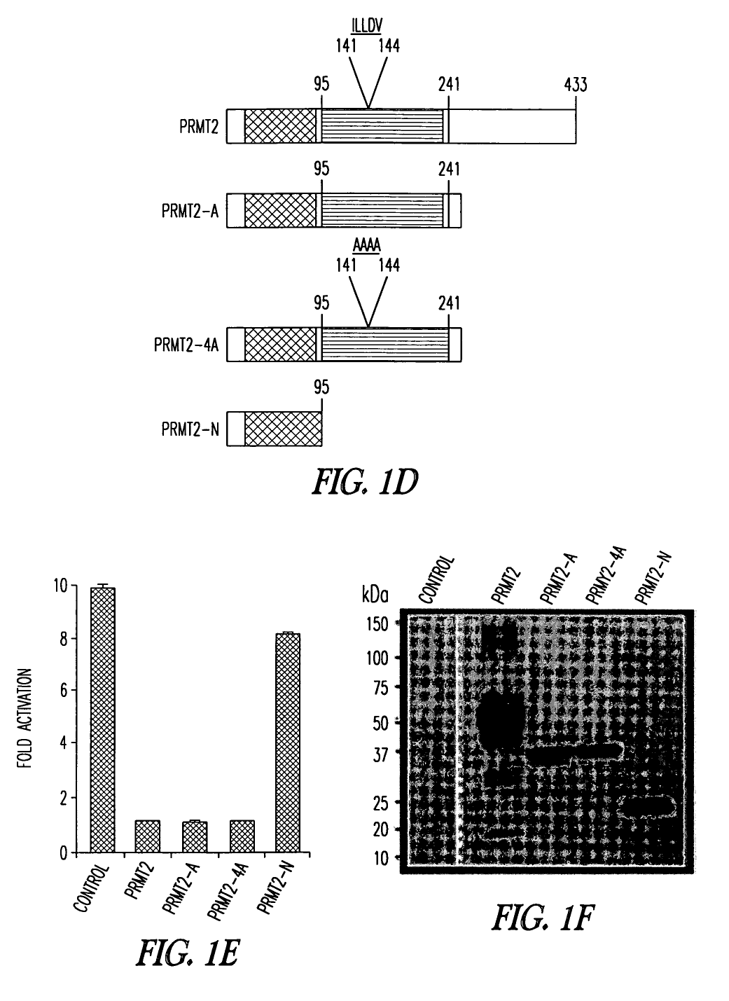

N-methylation of proteins at arginine residues is catalyzed by the PRMT (protein arginine methyltransferase) family of methyltransferases. Chiao et al. Proc. Natl. Acad. Sci. USA 91, 28-32 (1994). Among the five arginine methyltransferases, PRMT1, 2, and 3 share similar structural motifs (FIG. 1A). The S-adenosyl methionine (Ado-Met) binding motifs of PRMT1, PRMT-2 and PRMT3 are related to those found in nucleic acid and small molecule methyltransferases. Chen, D. et al. Science 284, 2174-2177 (1999); Kagan et al. Arch Biochem. Biophys. 310, 417-427 (1994); Lin et al. J Biol. Chem. 271, 15034-15044 (1996); Abramovich et al. EMBO J 16, 260-266 (1997); Katsanis et al. Mammalian Genome 8, 526-529 (1997); Tang et al. J Biol. Chem. 273, 16935-16945 (1998); Scott et al. Genomics 48, 330-340 (1998); Pollack et al. J Biol. Chem. 274, 31531-31542 (1999). Other less homologous protein methyltransferases, such as CARM1 (PRMT4) and JBP1 (PRMT5), also have such an S-adenosyl methionine binding motif.

To determine whether PRMT-1, 2 or 3 could affect NF-κB function, their potential to regulate effects on transcription of the human immunodeficiency virus type 1 (HIV-1) was examined. Transient co-transfections were performed using PRMT1, PRMT2 and PRMT3 expression plasmids with an HIV-1 reporter plasmid in the human renal epithelial cell line, 293T.

While PRMT1 stimulated HIV-1 transcription about 10-fold, PRMT-2 inhibited HIV-1 transcription about 50-fold and PRMT3 did not affect transcription of the HIV-1 reporter plasmid (FIG. 1B). For PRMT-2, a statistically significant effect was noted at 2.5 μg and 5 μg concentrations (p<0.001, at 5 μg relative to the vector control by Student's t-test). PRMT4 and PRMT5 failed to inhibit NF-κB transcription specifically. Similar results were obtained in other cell types (data not shown). These results suggest that PRMT-2 is unique among the PRMTs in its ability to inhibit HIV-1 transcription.

To map the domains responsible for inhibition of HIV transcription, truncation and point mutations were made in PRMT2 (FIG. 1D) and co-transfected with an NF-κB reporter in 293T cells. PRMT2-A represents an alternatively spliced form of PRMT2 found in the expressed sequence tag (EST) database. This isoform contains the first 218 amino acids of PRMT2 and differs from full length PRMT2 by the absence of the less conserved COOH-terminal domain. PRMT2-N was generated by introducing a stop codon after amino acid 95 before the putative Ado-Met domain of PRMT2. To analyze the role of the Ado-Met domain further, another mutant, PRMT2-4A, was prepared in which ₁₄₁ILDV₁₄₄ (SEQ ID NO:5) in this region were altered to four consecutive alanines to compare the effects of point mutations in this highly conserved region. Under conditions in which PRMT2, PRMT2-A and PRMT2-4A inhibited NF-κB activity, PRMT2-N did not (FIG. 1E), suggesting that a structural, but not necessarily a functional, methyltransferase domain is required for transcriptional inhibition.

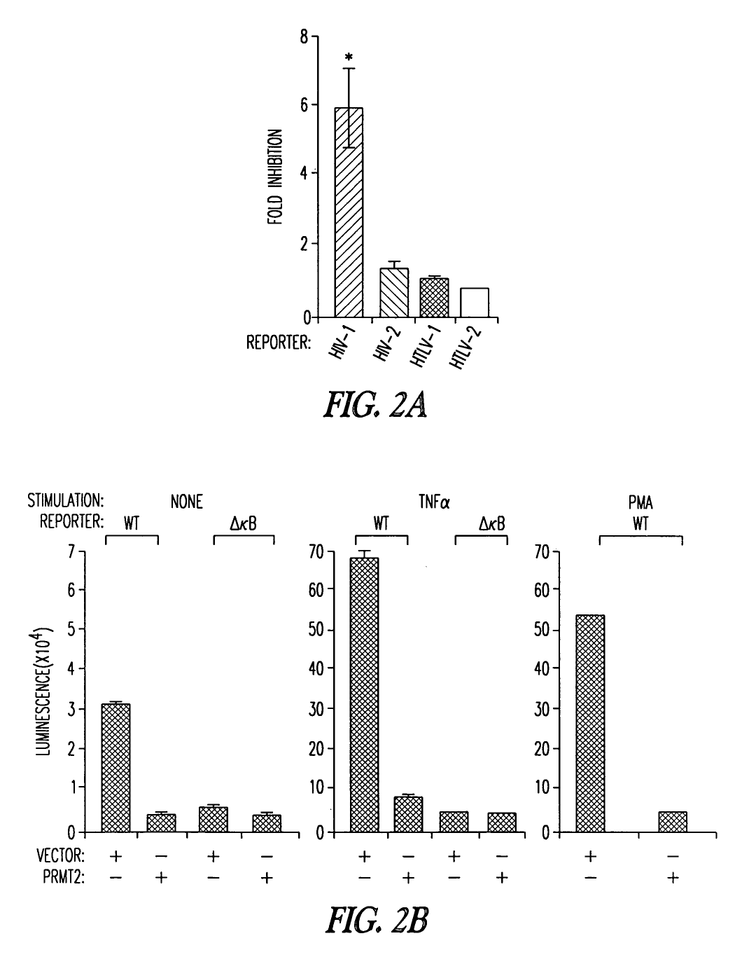

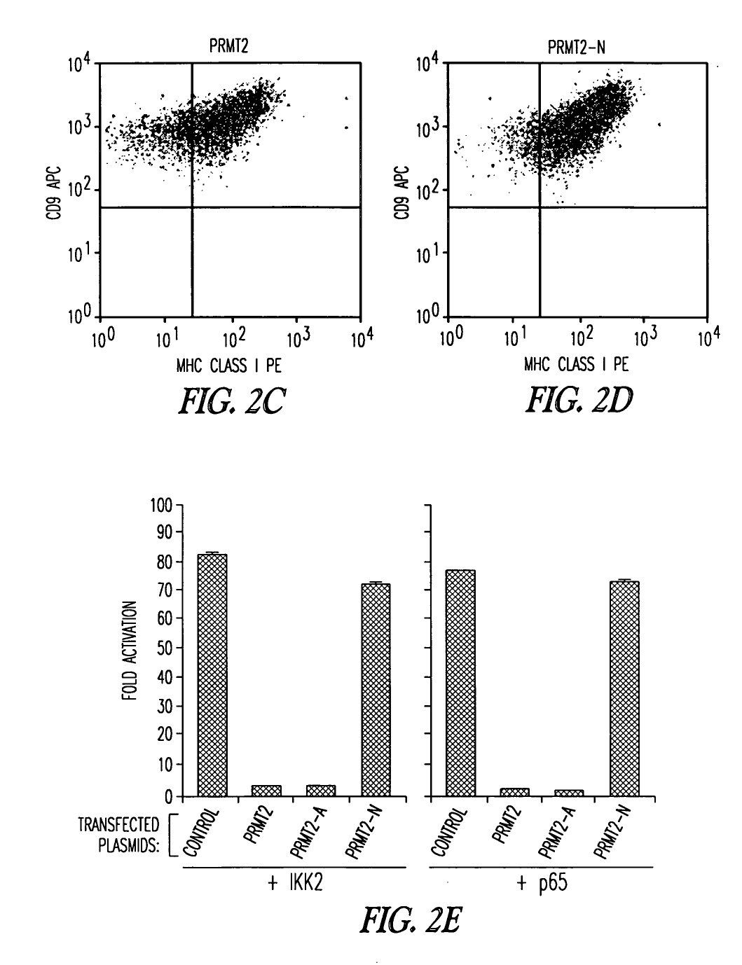

The HIV-1 LTR contains two highly conserved κB-binding sites that play an important regulatory role in HIV-1 gene expression (Nabel and Baltimore, Nature 326: 711-13 (1987)). To study the effect of PRMT2 on transcription, PRMT2-A was cotransfected with HIV-1, HIV-2, HTLV-1, or HTLV-2 reporter plasmids into 293 cells. Despite the presence of a single κB site in HIV-2, its expression shows greater dependency on Ets family transcription factors (Leiden et al., J. Virol. 66: 5890-97 (1992)). No significant reduction was seen with either HIV-2 or HTLV reporter plasmids, while HIV-1 CAT expression was substantially inhibited, documenting the specificity of PRMT2 for HIV-1 (FIG. 2A).

To determine its dependence on NF-κB, human immunodeficiency virus type 1 (HIV-1) reporter plasmids with wild-type (WT) or mutant (ΔκB) sites were co-transfected transiently with control or PRMT2 expression plasmids in the different cell lines. PRMT2 significantly inhibited both basal and TNF-α-dependent HIV-1 transcription from the wild-type but not the κB-mutant reporter in 293 renal epithelial cell lines (FIG. 2B, left and middle panels). The KB effect was dose-dependent and was also observed with other inducers of NF-κB, including phorbol myristic acid (PMA) (FIG. 2B, right panel). These results suggested that PRMT2 could block NF-κB activation from various stimuli.

PRMT2 was also able to modulate the expression of endogenous κB-regulated genes. PRMT2 transfection of 293T cells decreased endogenous MHC Class I cell surface expression by flow cytometry, in contrast to CD9, which is an NF-κB-independent gene (FIG. 2C-D).

The mechanism and site of action of PRMT2 in the NF-κB signaling pathway was further defined by co-transfection of PRMT2 and its mutants with different regulators in this pathway with an NF-κB reporter in 293T cells (FIG. 2D). PRMT2 and PRMT2-A inhibited both IKK2- and p65-induced NF-κB activity (FIG. 2E), while PRMT2N was unable to block this effect (FIG. 2E, right bars), suggesting that PRMT2 exerted its inhibitory action on nuclear NF-κB rather than by modulation of cytoplasmic IκB or the IκB kinase complex.

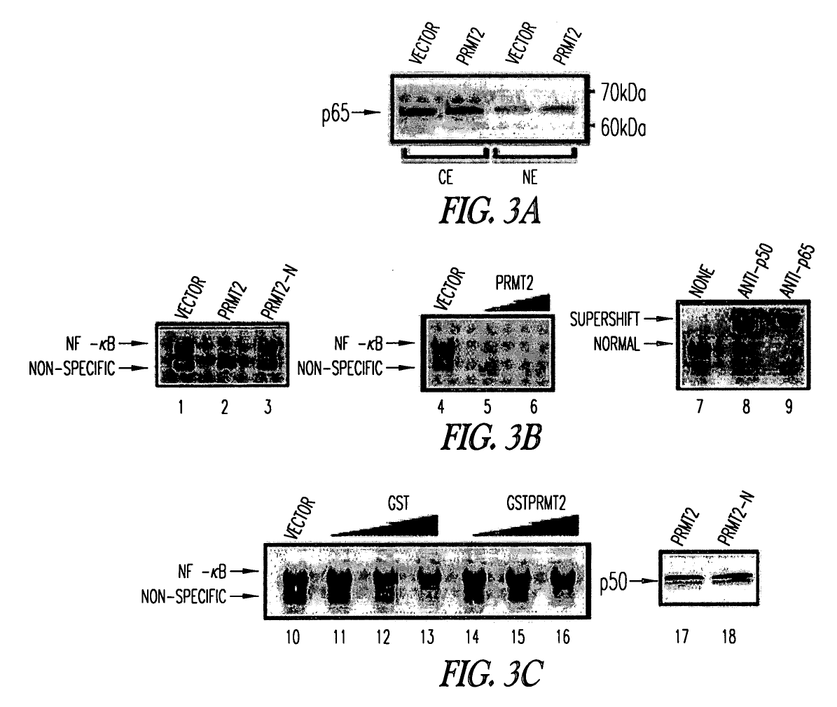

To investigate this mechanism further, p65 expression levels and cellular localization of RelA and IκB were examined. Immunoblotting for RelA in cytoplasmic and nuclear extracts from 293 cells transfected with PRMT2 revealed no effect on RelA protein levels or on its subcellular localization (FIG. 3A). Thus, PRMT2 appeared to affect RelA function without altering its nuclear accumulation, for example, by interfering with its DNA binding activity.

To determine whether PRMT2 can affect nuclear NF-κB DNA binding activity, PRMT2 was cotransfected into 293 cells with the NF-κB1 (p50) and RelA (p65) expression vectors. Analysis of nuclear extracts from transfected cells by mobility shift assays, using a consensus κB-binding site double-stranded oligonucleotide, showed that PRMT2 inhibited DNA binding of the p50/p65 complex in a dose-dependent manner (FIG. 3B, left and middle; lanes 2, 5, 6). In contrast, the inactive PRMT2-N mutant did not affect NF-κB DNA binding (FIG. 3B, left; lane 3). The nature of these complexes was confirmed by supershifts with antibodies directed against p50 and p65 (FIG. 3B, right, lanes 8 and 9).

To examine whether PRMT2 directly affected NF-κB DNA binding, a recombinant glutathione-S-transferase (GST) PRMT2 fusion protein, GST-PRMT2, was added to the gel-shift reaction mixture. No decrease in DNA binding over GST control was observed (FIG. 3C; lanes 14, 15, 16), suggesting that the inhibition of NF-κB DNA binding in PRMT2-transfected extracts was indirect. Because p50/p65 dimerization is important for efficient NF-κB DNA binding (Sen and Baltimore, 1986), PRMT2 might inhibit DNA binding by antagonizing p50/p65 complex formation. To test this possibility, p50/p65 complexes were immunoprecipitated from PRMT2-transfected 293 cell nuclear extracts, using an anti-p65 antibody. Western blotting for p50 showed that equal amounts of p50 co-immunoprecipitated from cells transfected with PRMT2 or PRMT2-N (FIG. 3C, right panel, lanes 17, 18), suggesting that decreased NF-κB DNA binding in PRMT2-transfected cell extracts was not due to interference with p50/p65 dimerization. In this assay PRMT2 also did not affect interactions of p65 with p300 and the general transcriptional machinery (supplemental FIG. 2A), nor did it catalyze the methylation of histones (supplemental FIG. 2B), p65, p50, IκB, hnRNPU, and CRM1 in both bacterially purified and cell extract immunoprecipitated PRMT2 in an in vitro methyltransferase assay (data not shown). Whole cell hypomethylated extracts from PRMT2-transfected cells showed minimal changes in methylation when incubated in vitro with [methyl-³H]S-adenosyl-L-methionine over control while PRMT1-transfected extracts were hypermethylated (supplementary FIG. 2C). Taken together, these data suggest that the methyltransferase function of PRMT2 is not necessary for inhibiting NF-κB activity.

Newly synthesized IκB-α can be detected in the cytoplasm but also in the nucleus, where it associates with NF-κB/RelA complexes. As newly synthesized IκB-α accumulates in the nucleus, there is a progressive reduction of both NF-κB DNA binding and NF-κB-dependent transcription (Arenzana-Seisdedos et al., J. Cell Sci. 110: 369-78 (1997)), presumably by export of NF-κB-IκBα complexes from the nucleus (Arenzana-Seisdedos et al., Mol. Cell. Biol. 15: 2689-96 (1995); Rodriguez et al., J. Biol. Chem. 274: 9108-15 (1999); Tam et al., Mol. Cell. Biol. 20: 2269-84 (2000)). PRMT2 could therefore potentially affect nuclear IκB-α levels, resulting in decreased NF-κB DNA binding.

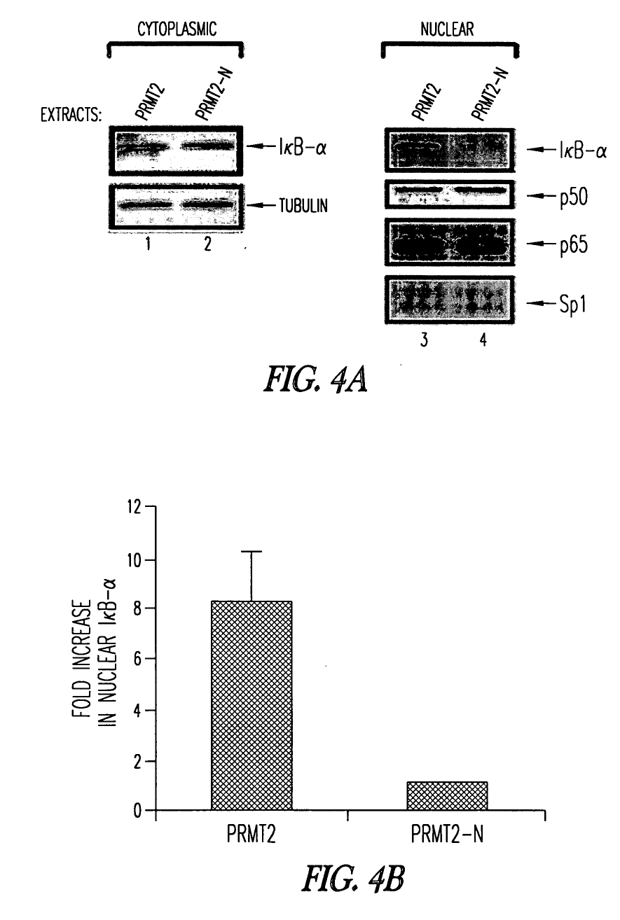

To examine whether PRMT2 increased nuclear IκB-α levels, nuclear and cytoplasmic extracts were prepared from PRMT2 or inactive, PRMT2-N transfected 293 cells. Immunoblotting for IκB-α and RelA proteins in the 2 fractions revealed no significant changes in the levels of cytoplasmic IκB-α (FIG. 4A, left panel, lanes 1 vs. 2) or nuclear p50 and RelA (p65) levels (FIG. 4A, right panel, lanes 3 vs. 4), but a distinct increase in the amount of nuclear IκB-α was observed in PRMT2-transfected cells compared to the functionally inactive PRMT2-N mutant control (FIG. 4A, right panel, lanes 3 vs. 4, and FIG. 4B; p<0.01, PRMT2 compared to the mutant PRMT2-N using Student's t-test) in cells that had been stimulated with TNF-α. This increase in the nuclear accumulation of IκB-α therefore appeared to be responsible for the PRMT2-mediated inhibition of NF-κB DNA binding and NF-κB-dependent transcription.

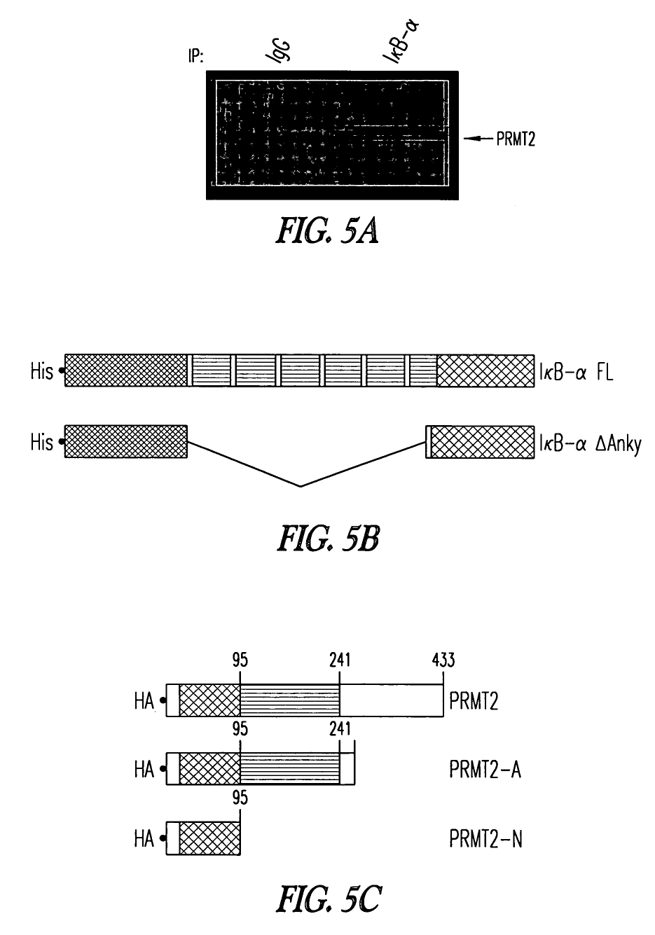

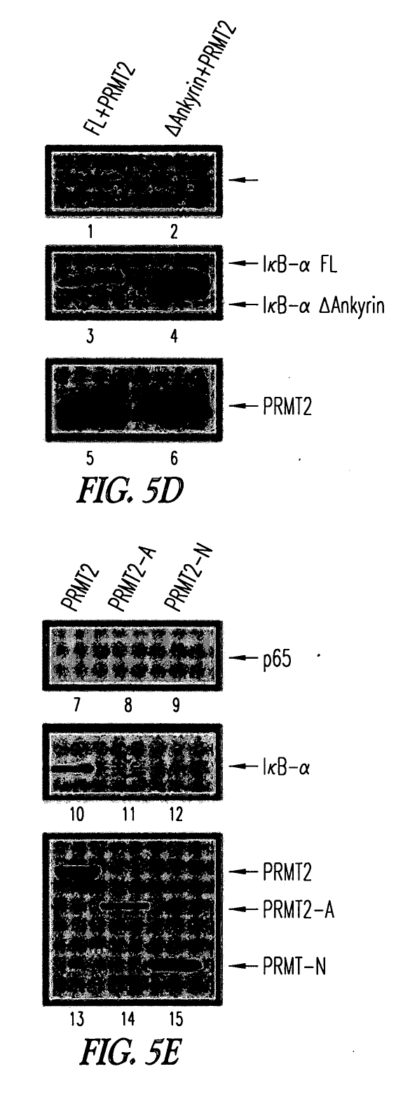

A polyclonal antibody to recombinant PRMT2 was used to examine the association between endogenous PRMT2 and IκB-α in vivo. Immunoprecipitation of IκB-α from NIH3T3 cell extracts with a control or anti-IκB-α antibody followed by immunoblotting with antibody to PRMT2 revealed that PRMT2 interacted with endogenous IκB-α (FIG. 5A). The domain of IκB-α required for association with PRMT2 was mapped using in vivo immunoprecipitation assays where HA-tagged PRMT2 was coexpressed with truncation mutants of His-tagged IκB-α (FIG. 5B) in 293 cells. The ankyrin domain was both necessary and sufficient for this association (FIG. 5D, lanes 1 and 2). The domain of PRMT2 that interacted with endogenous IκB-α was mapped by immunoprecipitation following expression HA-tagged PRMT2 truncation mutants (FIG. 5C). IκB-α interacted with PRMT2 and PRMT2-A (FIG. 5E, lanes 10 and 11) but did not associate with PRMT2-N (FIG. 5E, lane 12), indicating that the Ado-Met domain is necessary to promote IκB-α binding. When the ratios of PRMT2 or PRMT2-A binding to IκB-α were compared, both interacted with IκB-α with similar affinity. PRMT2 or the mutants did not interact with endogenous p65 (FIG. 5E, lanes 7, 8, 9).

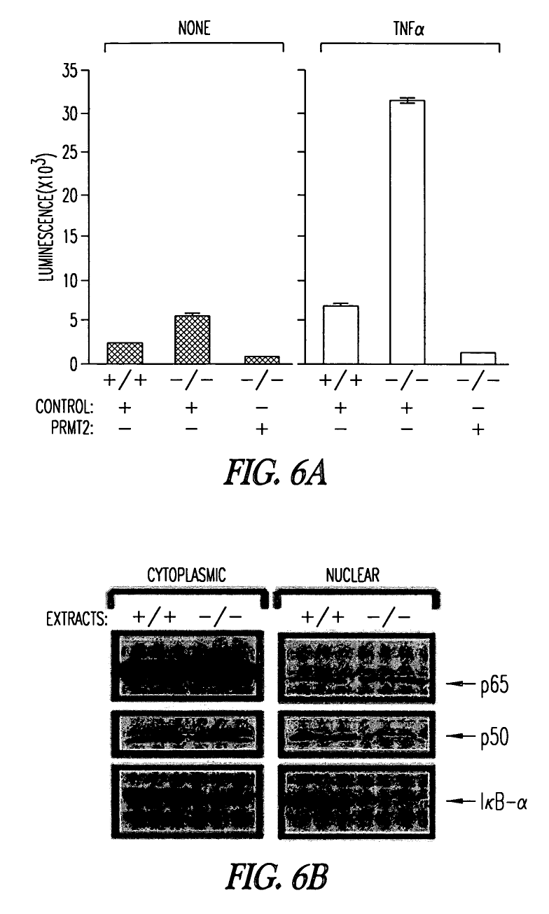

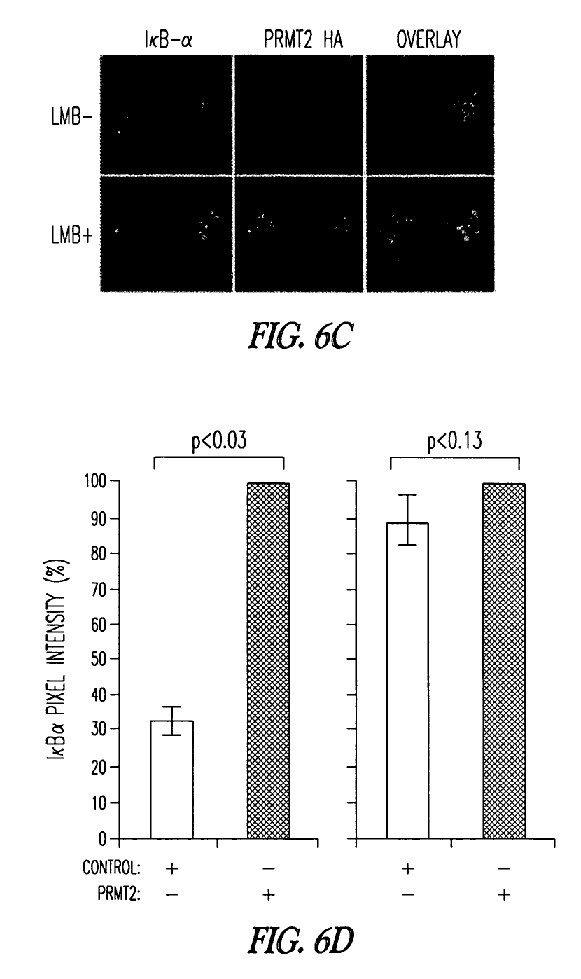

To determine whether similar effects would be observed in non-transformed cell lines with physiological levels of protein, NF-κB inducibility was analyzed in mouse embryonic fibroblasts (MEFs) derived from PRMT2 null mice (T. Y. et al., manuscript in preparation). A κB luciferase reporter construct was transfected with control or PRMT2 expression plasmid into WT and PRMT2^{-/-} MEFs and incubated in the presence or absence of TNF-α. Compared to wild type cells, and consistent with the transfection results in 293 cells, PRMT2^{-/-} MEFs were more responsive to NF-κB induction by TNF-α (FIG. 6A). Complementation of PRMT2^{-/-} MEFs with PRMT2 completely abolished NF-κB induction by TNF-α (FIG. 6A).

IκB-α and p65 levels were examined in cytoplasmic and nuclear extracts from control and PRMT2^{-/-} MEFs. Immunoblotting for p65, p50 and IκB-α in the 2 fractions revealed no significant changes in the levels of cytoplasmic IκB-α (FIG. 6B, middle panel) or p50 or RelA (p65) levels (FIG. 6B, top panel), but a distinct decrease in the amount of nuclear IκB-α was observed in PRMT2^{-/-} compared to control MEFs (FIG. 6, right middle panel). NF-κB DNA binding and NF-κB-dependent transcriptional activation is reduced by accumulation of newly synthesized IκB-α in the nucleus (Arenzana-Seisdedos et al., 1997). NF-κB-IκBα complexes are exported from the nucleus to the cytoplasm by CRM1 (Arenzana-Seisdedos et al., 1995; Rodriguez et al., 1999; Tam et al., 2000) and this nuclear export can be blocked by leptomycin B (LMB) (Ossareh-Nazari et al., Science 278: 141-44 (1997); Tam et al., 2000; Huang and Miyamoto, Mol. Cell. Biol. 21: 4737-47 (2001)). To understand the role of PRMT2 in promoting nuclear IκBα accumulation, PRMT2^{-/-} fibroblasts were transfected with an HA-tagged PRMT2 expression vector. 36 hrs after transfection the cells were treated with TNF-α for 30 min. The media was then removed and cells were incubated for an additional 30 minutes in the presence or absence of LMB. Cells were fixed, permeabilized and stained for IκBα (FIG. 6C, left panel) and HA (PRMT2, FIG. 6C, middle panel). Confocal microscopy performed on the cells showed IκB-α accumulation in the nucleus in the presence of PRMT2 (FIG. 6C, top left and right panel) which did not change in the presence of LMB (FIG. 6C, bottom left and right panel). To demonstrate the effect of PRMT2 further, nuclear IκBα (FIG. 6D) was quantified in PRMT2^{-/-} fibroblasts and PRMT2^{-/-} fibroblasts complemented with PRMT2 in the presence or absence of LMB. LMB promoted nuclear accumulation of IκBα in the absence of PRMT2, and transfection of PRMT2 exerted the same effect. Together, these data suggest that PRMT2 inhibits the nuclear export of IκB-α through a LMB-sensitive, CRM1 pathway.

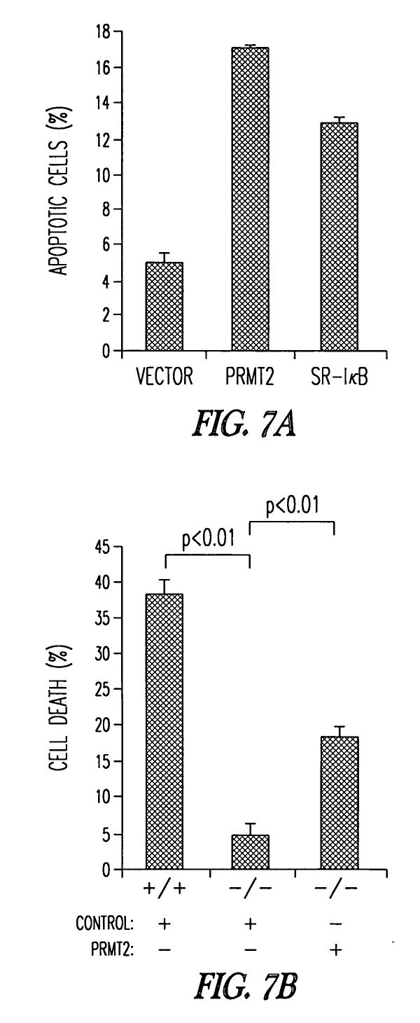

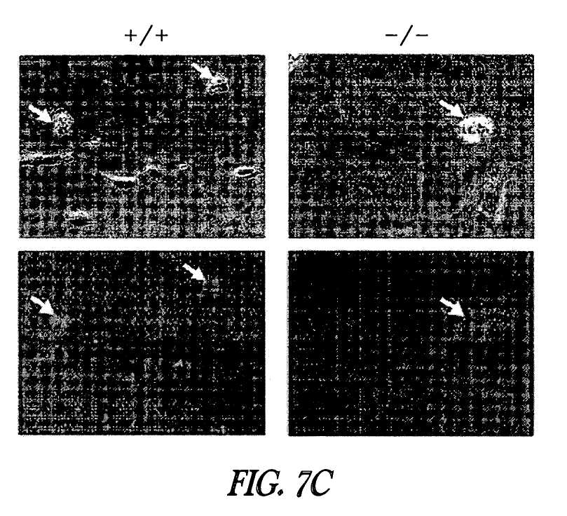

Because PRMT2 inhibits NF-κB activity, which can regulate apoptosis in some cell types (Beg and Baltimore, Science 274: 782-84 (1996); Wang et al., Science 274: 784-87 (1996); van Antwerp et al., Science 274: 787-89 (1996)), the ability of PRMT2 to independently regulate programmed cell death was examined. Transfection of PEMT2 into 293 cells increased their susceptibility to TNF-induced cell death, to levels comparable to those observed by a mutant, stabilized, or superrepressor, IκB (SR-IκB) (Beg and Baltimore, Science 274: 782-84 (1996); Wang et al., Science 274: 784-87 (1996); Wang et al. Nat. Med. 5: 412-17 (1999)) (see FIG. 7A). To evaluate the effect of PRMT2 on programmed cell death, wild type, PRMT2 knockout MEF or knockout MEF cells complemented with PRMT2 were exposed to etoposide, a DNA-damaging agent with pro-apoptotic activity. Wild type and PRMT2 complemented MEFs displayed a substantial increase in etoposide-induced cell death and annexin V staining compared to PRMT2-deficient cells (FIGS. 7B and 7C). These data indicate that PRMT2 promotes apoptosis.

Therefore, this Example shows that PRMT2 inhibits NF-κB-dependent transcription and promotes apoptosis. PRMT2 exerted this effect by blocking nuclear export of IκB-α through a leptomycin-sensitive pathway, increasing nuclear IκB-α and decreasing NF-κB DNA binding. The highly conserved S-adenosylmethionine binding domain of PRMT2 mediated these effects. PRMT2 also rendered cells susceptible to apoptosis by cytokines or cytotoxic drugs, likely due to its effects on NF-κB. Mouse embryo fibroblasts from PRMT2 genetic knockouts showed similar alterations of NF-κB activity and decreased susceptibility to apoptosis compared to wild type or complemented cells. Taken together, these data suggest that PRMT2 inhibits cell activation and promotes programmed cell death through this κB-dependent mechanism.

### EXAMPLE 2

### PRMT-2 Binds RB and Regulates E2F Function

This Example shows that PRMT-2 interacts with RB and can modulate E2F function, whereas PRMT1, PRMT3, and PRMT4 do not.

### Materials and Methods

Plasmids

Human PRMT1, PRMT-2, PRMT3, PRMT4, and mouse PRMT-2 cDNA were cloned by RT-PCR using total RNA extracted from Jurkat cells and mouse cardiac tissue, respectively. The following primers pairs were used for PCR: 5′-AAGGATCCGCGAACTGCAT CATGGAGAA-3′ (SEQ ID NO:19) and 5′-A AAAGCTTAAACCGCCTAGGAACGCTCA-3′ (SEQ ID NO:20) for human PRMT1; 5′-AAGTCGACGCCATGGC AACATCAGGTGACTGT-3′ (SEQ ID NO:21) and 5′-AAGCGGCCGC TTATCTCCAGATGGGGA AGACTT-3′ (SEQ ID NO:22) for human PRMT-2; 5′-AAGATATCGC CATGGACGAGCCA GAACTGTCGGACAGCGGGGACGAGGCCGCCTGGGAGGATGAGGACGAT-3′ (SEQ ID NO:23) and 5′-AATCTAGATTACTGGAGACCATAAGTTTG AGTTG-3′ (SEQ ID NO:24) for human PRMT3; 5′-AAGAATTCT AAGATGGCAGCGGCGGCA-3′ (SEQ ID NO:25) and 5′-AAAA GCTTCTAACTCCCATAGTGCATGG TGTT-3′ (SEQ ID NO:26) for human PRMT4; 5′-AAGGATCCAGCCCCA GTTATGAGACATGAT-3′ (SEQ ID NO:27) and 5′-AAAAGCTT CTTCTTTCACTGAGATGCATGC-3′ (SEQ ID NO:28) for mouse PRMT-2.

PRMT-2 deletion mutant were generated by PCR. Wild type PRMTs and mutant PRMT-2 were subcloned into the following plasmids: pcDNA-3 plasmid (Invitrogen) for in vitro translation, pVR1012 (a eukaryotic expression vector driven by CMV immediate-early promoter with enhancer and intron) for transient transfection. Danthinne et al. (1998) J Virol, 72, 9201-7.

The PRMT-2 motif I mutant was generated from the wild type pVR012 PRMT-2 construct using the Stratagene Quickchange™ Site-Directed Mutagenesis kit. CMV-RB was a kind gift from Dr. Karen Vousden and coding region of RB cDNA was subcloned into pGEX-6P (Amersham/Pharmacia) for generation of GST-RB. Luciferase reporter G5E4T-Luc was generated by subcloning a 187 bp XhoI-KpnI fragment (containing five tandem GAL4 sites and an adenovirus E4 TATA box) from pG5E4TCAT (Emami and Carey (1992) Embo Journal, 11, 5005-5012) into multi-cloning site of pGL3-Basic (Promega). The E2F4B-Luc vector is described in Dick et al. (2000) Molecular and Cellular Biology, 20, 3715-3727. pHKGAL4 E2F1 AD was obtained from T. Kouzarides. CMV E2F1 was obtained from W. G. Kaelin Jr. and K. Helin.

Cell Culture, Transfection, and Antibodies

HeLa cells, 293 cells, U2OS cells and Saos 2 cells were grown in DMEM supplemented with 10% FBS. PRMT-2^{-/-} mouse embryo fibroblasts (MEFs) and wild type PRMT-2^{+/+} MEFs were prepared from day 13.5 embryos and maintained in DMEM supplemented with 10% FBS.

FuGene 6 Transfection Reagent (Roche) was used for transfection according to the directions from the manufacturer. Monoclonal antibodies to HA (12CA5 and 3F10: Roche), RB (G3-245: Pharmingen), E2F1 (KH95 Pharmingen), BrdU (FITC-labeled clone 3D4: Pharmingen), Flag (M2: Sigma), polyclonal antibodies to RB (C-15: Santa-Cruz) and actin (A2066:Sigma) were used. A PRMT-2 5F8 antibody was raised in mice immunized with a peptide having the sequence CDMRTVQVPDLETMR (SEQ ID NO:29), corresponding to amino acid 322-339 of mouse PRMT-2 (A&G Pharmaceutical, Inc.).

Protein Production, In Vitro Binding Assay, Immunoprecipitation and Western Blot Analysis.

The crude cell lysate of the GST-fusion proteins was prepared and purified according to the manufacture's protocol (Amersham/Pharmacia). [³⁵S]-methionine-labeled protein was produced by in vitro transcription/translation using the TNT T7-coupled reticulocyte lysate system (Promega). [³⁵S]-methionine-labeled protein and GST-fusion protein bound to glutathione sepharose 4B beads were incubated in 500 μl of HNE buffer (50 mM Hepes, pH 7.8, 150 mM NaCl, 1 mM EDTA, 1 mM DTT, 1% NP40, and 1× protease inhibitor mix (Complete: Roche)) at 4° C. for 1 hour. Beads were washed four times with HNE buffer, and then analyzed on an SDS-PAGE gradient gel (4-15%). For the detection of endogenous PRMT-2 interaction with RB, MEFs were lysed with WCL buffer (50 mM Hepes, pH 7.8, 400 mM NaCl, 2 mM EDTA, 1 mM DTT, 0.2% NP40, 10% Glycerol, 20 mM α-glycerophosphate, 5 mM NaF, 0.1 mM NaVO₄, and 1× protease inhibitor mix). Then, 1 mg of whole cell extracts were incubated with HNE buffer without DTT and 3 μg of antibodies as indicated. The immune complexes were isolated using protein G beads, and washed four times with HNE buffer. The precipitated protein was boiled with SDS-loading buffer and resolved on SDS-PAGE. For immunoprecipitation of HA-tagged protein, transfected cells were lysed with RIPA buffer (phosphate buffer saline containing 1% NP40, 0.5% sodium deoxycholate, 0.1% SDS, and 1× Complete), and 100 μg of whole cell lysates were immunoprecipitated with anti-HA antibodies and washed in RIPA buffer. Western blot analysis was performed as described in Tanner et al. (1998). Circ Res, 82, 396-403.

Immune Complex Methylation Assay

For methylation assay, 293 cells transfected with either PRMT-2 or mutant PRMT-2 were lysed and immunoprecipitated with anti-HA antibodies (12CA5) in RIPA buffer. The precipitates were washed three times with RIPA buffer and methylation buffer (20 mM Tris-Cl, pH 8.0, 200 mM NaCl, 4 mM EDTA). The methylation reaction on histone H2A was performed as described in Chen et al. (1999) Science 284, 2174-2177. Labeled proteins were resolved on 15% SDS-PAGE and subjected to fluorography.

E2F Reporter Assay

HeLa cells, U2OS cells, and Saos2 cells were seeded at a density of 5×10⁴ per well in 24-well plates. Cells were transfected 24 hours later with the indicated plasmid and with 20 ng of pRL-TK (Promega) as an internal control to normalize transfection efficiency. The amount of CMV promoter in the transfection was kept constant using empty vector. Cells were harvested at 30 or 36 hour after transfection and luciferase activity was analyzed by Dual-Luciferase Reporter Assay System (Promega). Luciferase activity was measured as a ratio of Firefly to Renilla. To study endogenous E2F activity in MEFs, PRMT-2^{+/+} or PRMT-2^{-/-} MEFs were seeded at a density of 3×10⁵ per well in 6-well plates. Cells were transfected 24 hours later with 3 μg of E2F4B-Luc and 10 ng of pRL-TK plasmid. Cells were harvested at 36 hour after transfection and analyzed for luciferase activity as described above.

BrdU Incorporation and Flow Cytometry Analysis

After pulse labeling for 1 hour with 10 M of BrdU, cells were detached with trypsin, fixed in 70% ethanol overnight, and treated with 2M HCl and 0.5% Triton X-100 for 30 min at room temperature followed by 0.1M sodium tetraborate for 3 minutes at room temperature. Duplicate samples were stained with FITC-conjugated anti-BrdU antibody (clone 3D4: Pharmingen) and FITC-conjugated isotype control antibody (MOPC-21) for 40 minutes, respectively. Then the samples were incubated with PBS containing 5 ug/ml propidium iodide, 5U RNase A (Sigma), 0.5% Tween 20. PBS washes were performed between each step. BrdU incorporation and DNA content were analyzed by flow cytometry.

Construction of PRMT-2 Targeting Vector

Mouse genomic clone containing PRMT-2 locus was isolated from a 129/SV mouse BAC library using mouse PRMT-2 cDNA as a probe (Incyte Genomics, Inc.). The PRMT-2 locus is located in mouse chromosome 10 (Cole et al. (1998) Genomics, 50, 109-11), and its DNA sequence is available from GeneBank database (Accession No. AC006507). The following genomic fragments were obtained by PCR from the genomic clone: a 1478 bp fragment containing exon 3 and a part of exon 4 (nt. 86002-87480 from AC006507) as the short arm; 6408 bp fragment containing a part of exon 6 and exon 7 (nt.91709-98116 from AC006507) as the long arm. A point mutation was introduced in the short arm to create a G119→stop codon mutation at exon 4. Then the short arm and the long arm were subcloned into Hpa I site and EcoRI-Sal 1 site of pKO Scramble 909 (Stratagene). A neomycin cassette from pKO SelectNeo (Stratagene) and a thymidine kinase (TK) cassette from pKO SelectTk (Stratagene) were subcloned into the Asc I site and the Rsr II site of the pKO Scramble 909, respectively.

Generation of Targeted ES Cells and PRMT-2^{-/-} Mice

The PRMT-2 targeting vector was linearized and electroporated into D3 ES cells. Clones doubly resistant to G418 (300 g/ml) and Gancyclovir (0.5 g/ml) were tested for homologous recombination by Southern blot analysis. DNA from ES cells was digested with EcoRI and two genomic probes (5′ probe: nt. 84051-85095; 3′ probe: nt. 101519-102645 from AC006507) were used for Southern hybridization to confirm homologous recombination. Two ES cell clones were used to produce chimeras with >90% agouti coats.

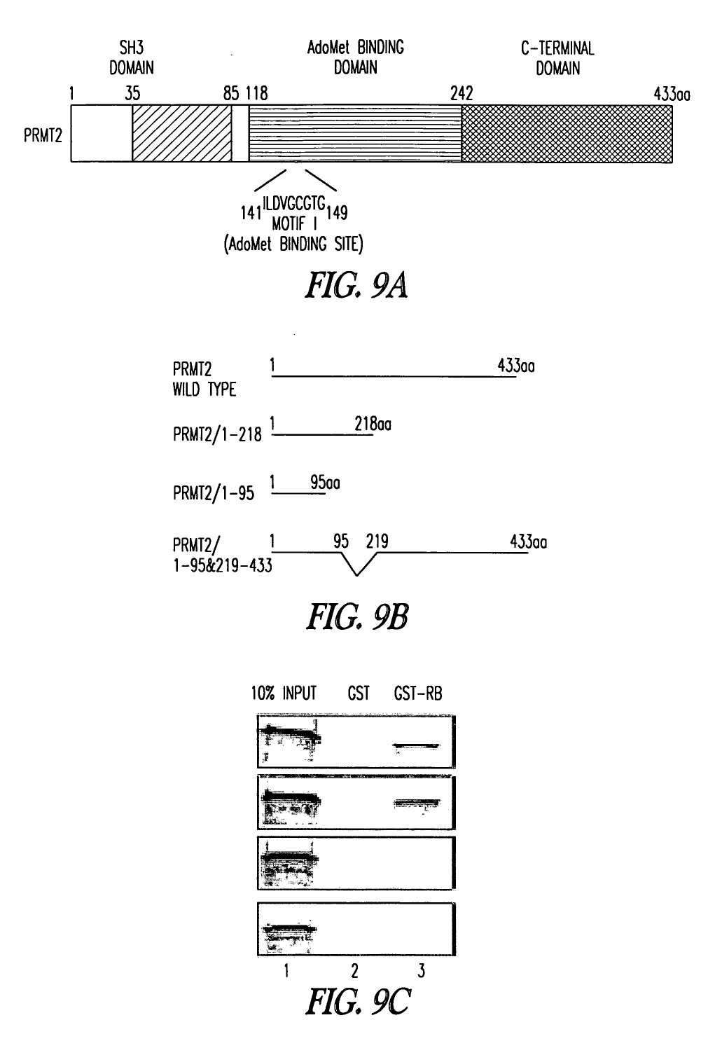

Male chimeras from both clones produced F1 agouti animals, 50% of which were F1 heterozygotes. Male and female F1 heterozygotes identified by Southern blot analysis were interbred to produce F2 progeny. A genomic PCR assay (FIG. 9C) was then used for subsequent genotyping using the common primer (primer b) 5′-CTGAGGTATTACCAGCAGA CA-3′ (SEQ ID NO:30), the wild type allele specific primer (primer a) 5′-CTCTCTGATGCAGGTCTAC-3′ (SEQ ID NO:31), and the mutant allele specific primer (primer c) 5′-CCGGTGGATGTGGAATG TGT-3′ (SEQ ID NO:32).

### Results

PRMT-2 Interacts with RB In Vitro and In Vivo

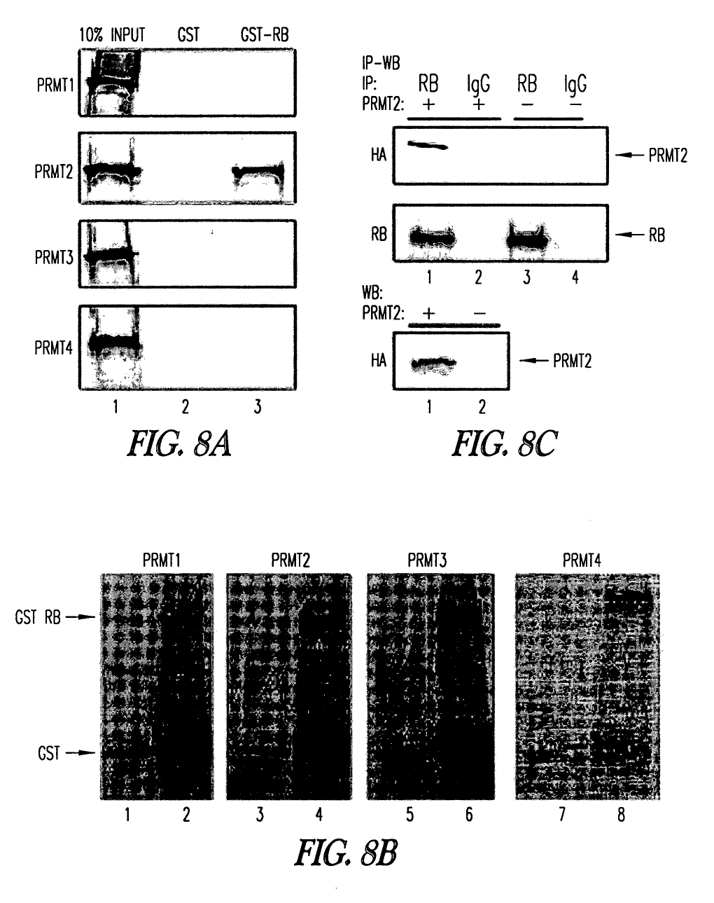

To test whether PRMT family members interact with RB, S³⁵-labeled in vitro translated PRMT1, PRMT2, PRMT3, and PRMT4 (CARM1) were incubated with GST-RB fusion proteins or control GST, and co-precipitated labeled proteins were resolved by SDS-PAGE. As shown in FIG. 8A, PRMT2 directly interacts with RB, but not with PRMT1, PRMT3, and PRMT4. An equal amount of GST-RB and GST were loaded in each lane, verified by Coomassie staining (FIG. 8B).

The interaction of PRMT2 with RB was then analyzed in vivo. HA-tagged PRMT2 expression vectors or control vectors were transfected into 293 cells, and immunoprecipitation of endogenous RB was followed by a Western blot analysis using an anti-HA antibody. PRMT2 directly interacted with RB in contrast to control vectors (FIG. 8C).

PRMT2 Interacts with RB Through the Ado-Met Binding Domain

Human PRMT2 has SH3, AdoMet and C-terminal domains, which have been deduced from amino acid sequence comparison with other PRMTs (Zhang et al. EMBO J. 19: 3509-19 (2000); Weiss et al. Nat. Struct. Biol. 7: 1165-71 (2000) (FIG. 9A). To determine the PRMT2 domain responsible for its interaction with RB, a series of deletion mutants in human PRMT2 were constructed (FIG. 9B). PRMT2(1-218) lacks the C-terminal domain but retains the SH3 domain and a large part of the AdoMet binding domain. PRMT2(1-95) lacks the C-terminal and AdoMet binding domains but retains the SH3 domain. PRMT2(1-95&219-433) is an internal deletion of amino acid 96-218, causing deletion of the AdoMet binding domain and retention of the SH3 and C-domains.

S³⁵ labeled PRMT2(1-218) bound GST-RB at levels comparable to that of wild type PRMT2 (FIG. 2C). However, PRMT2(1-95) and PRMT2(1-95&219-433) did not bind GST-RB (FIG. 2C), indicating that PRMT2 interacts with RB through its AdoMet binding domain.

PRMT2 Represses E2F Transcriptional Activity in a RB-Dependent Manner

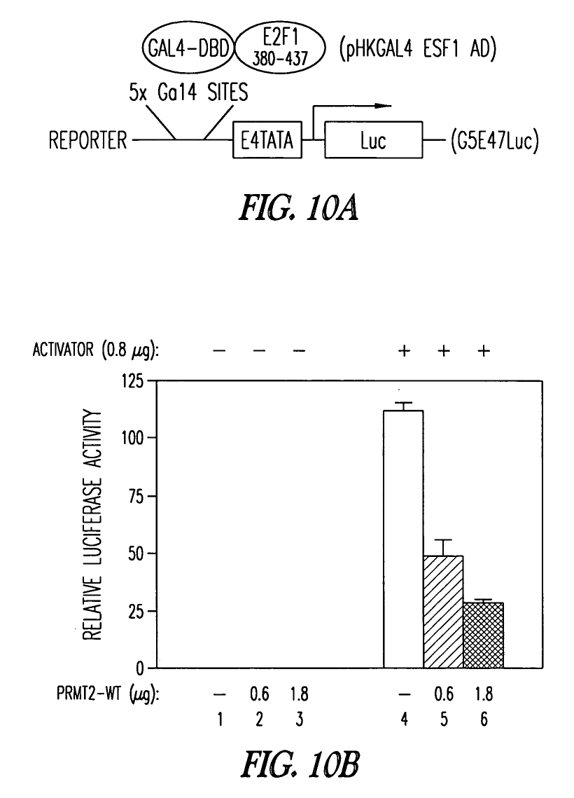

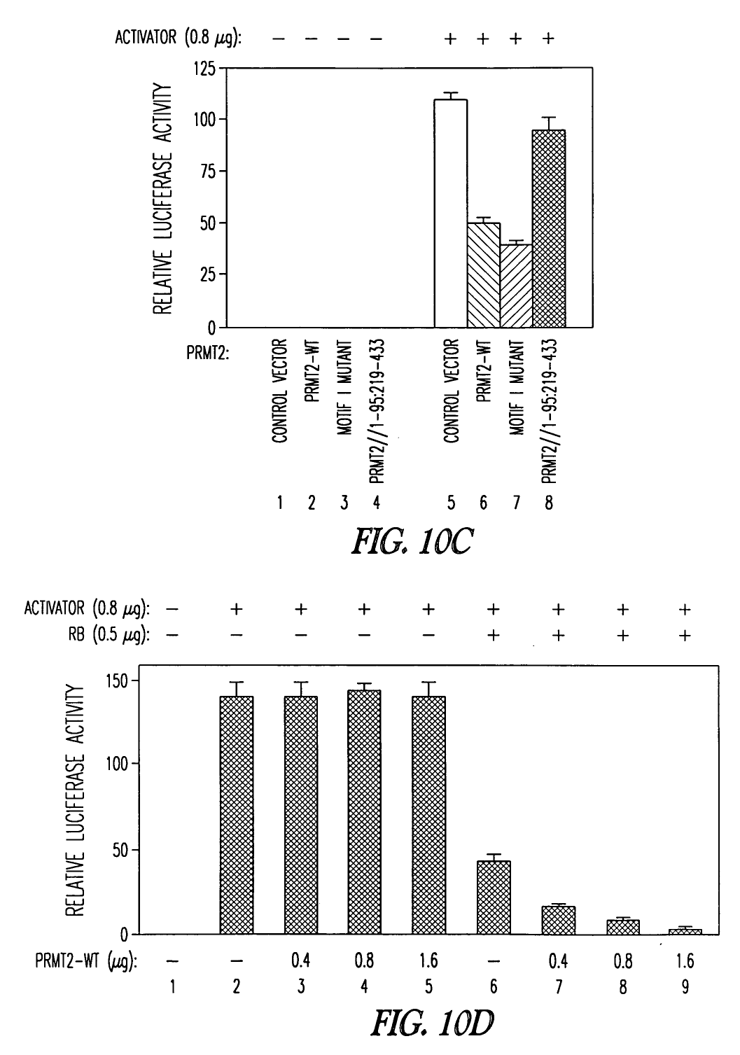

The E2F transcription factor is a major target of Rb. See, Dyson, Genes Dev. 12: 2245-62 (1998); Harbour et al. Genes Dev. 14: 2393-2409 (2000). To investigate whether PRMT2 regulates transcriptional activation by E2F, HeLa cells were transfected with a GAL4 luciferase reporter vector (G5E4T-Luc). An expression vector for a GAL4 DNA binding domain was fused to the E2F1 activation domain (pHKGAL4 E2F1-AD) as an activator (FIG. 10A). In the absence of the E2F1 activator, co-transfection of wild type PRMT2 did not activate the GAL4 promoter (FIG. 10B, bars 1-3). However, in the presence of the E2F1 activator, promoter activity increased more than 100 fold, compared to the absence of the activator (FIG. 10B, bars 4-6). In the presence of the E2F1 activator, co-transfection of PRMT2 repressed E2F1-induced promoter activity in a dose-dependent manner (FIG. 10B, bars 4-6). Similar results were observed in U2OS cells (data not shown).

To determine whether the methyltransferase activity of PRMT2 alters E2F repression, U2OS cells were transfected with the GAL4 luciferase reporter vectors, along with wild type PRMT2 vectors or mutant vectors, in the absence or presence of the E2F1 activator vectors. In the absence of the E2F1 activator, co-transfection of wild type PRMT2, its mutants, or control vectors did not activate the GAL4 reporter (FIG. 10C, bars 1-4). However, in the presence of the E2F1 activator, promoter activity increased more than 100 fold compared to the absence of the activator (FIG. 10C, bar 5). Co-transfection of the PRMT2 motif I mutant repressed E2F1 activity to a level comparable to that of wild type PRMT2 (FIG. 10C, bar 7). In contrast, co-transfection of PRMT2/1-95&219-433, which lacks the RB binding domain, failed to repress E2F1 activity (FIG. 10C, bar 8). S³⁵-labeled PRMT2 motif I mutants interacted with GST-RB at a level comparable to that of wild type PRMT2 (data not shown). These findings indicate that the AdoMet binding domain of PRMT2 is required for binding to RB and repression of E2F activity.

To further investigate whether E2F repression by PRMT2 is RB-dependent, Saos 2 cells, which lack functional RB, were transfected with the GAL4 reporter and E2F1 activator vectors. Co-transfection of PRMT2 with the E2F1 activator in these RB-deficient cells did not repress E2F1 activity (FIG. 10D, bars 1-5). However, when RB was co-transfected into the Saos 2 cells, along with PRMT2 and E2F1 activator vectors, E2F repression by PRMT2 in a dose-dependent manner was observed (FIG. 10D, bars 6-9), implying that RB is required for E2F repression by PRMT2.

PRMT2 Forms a Complex Formation with E2F and RB

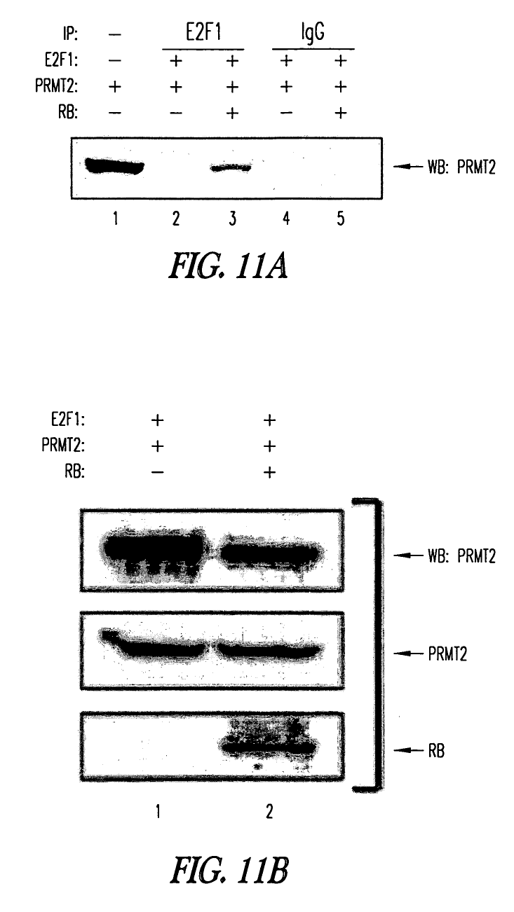

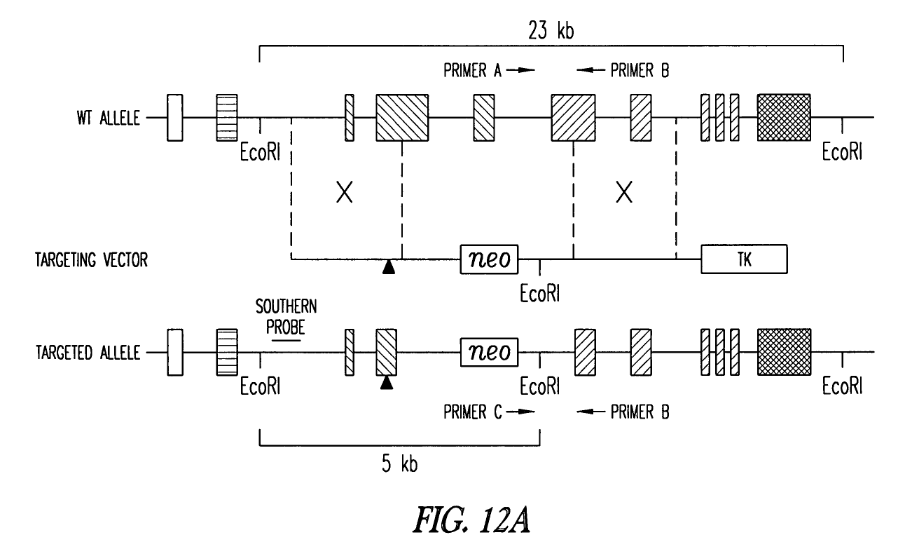

RB is required for E2F repression by PRMT2. The inventors hypothesized that PRMT2 could be recruited to E2F through its physical interaction with RB and function as a modulator of RB. To determine whether PRMT2 forms a complex with both RB and E2F1, these three expression vectors were co-transfected into RB negative Saos 2 cells. Cell lysates were immunoprecipitated with an E2F1 antibody, followed by measurements of PRMT2 by Western blot. A Western blot for PRMT2 served as a control (FIG. 11A, lane 1). In cells transfected with E2F1, PRMT2 and RB, PRMT2 co-immunoprecipitated with E2F1 (FIG. 11A, lane 3). However, in the absence of RB, PRMT2 did not immunoprecipitate with E2F1 (FIG. 11A, lane 2). Immunoprecipitations with IgG in the absence and presence of RB served as controls, respectively (FIG. 11A, lanes 4, 5). Expression levels of transfected E2F1, PRMT2, and Rb, and E2F1 in cell lysates are indicated by Western blot (FIG. 11B). These data suggest that PRMT2 forms a complex with E2F and RB and that RB is required for this interaction.

PRMT2 Directly Regulates Endogenous E2F Activity and Cell Cycle Progression

To investigate the endogenous interaction between PRMT2 and RB, PRMT2^{-/-} null mice we generated by homologous recombination. A PRMT2 targeting vector was constructed by replacing a portion of exon 4, 6, and all of exon 5 with a Neo^{R} cassette in the antisense orientation. A point mutation, generating a stop codon, was introduced at Gly119, such that RNA transcripts expressed from the mutant allele do not encode the AdoMet Binding domain and C-terminal domain (FIG. 12A). Homologous recombination was confirmed by Southern blot (FIG. 12B). PCR (FIG. 12C), and Northern blot analyses (FIG. 12D). PRMT2^{-/-} mice were viable and born with a normal Mendelian distribution. No gross abnormalities were apparent upon examination.

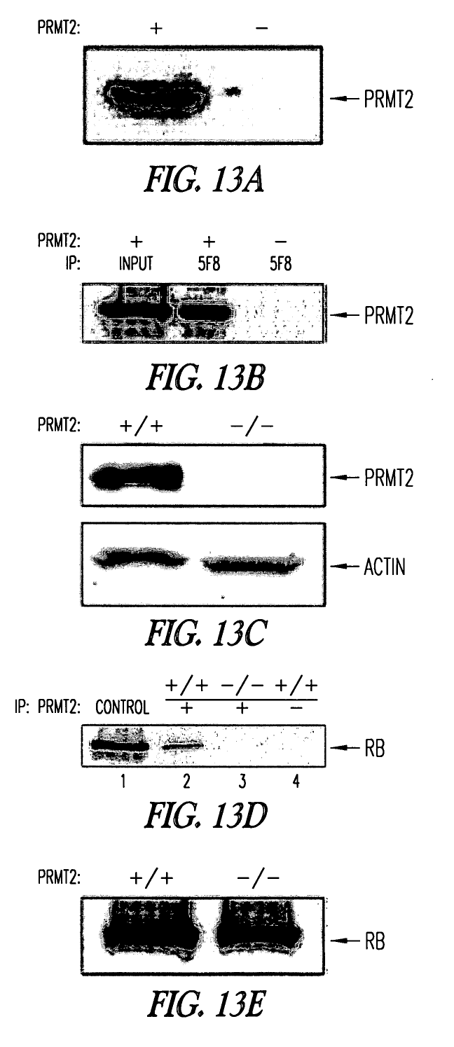

To further explore PRMT2 activity, a mouse monoclonal antibody was generated to PRMT2. This antibody recognized overexpressed PRMT2 in cell lysates by Western blot (FIG. 13A) and immunoprecipitation (FIG. 13B), and its specificity was confirmed in whole cell extracts from MEFs derived from PRMT2^{+/+} and PRMT2^{-/-} mice. An expected 55 kDa band was detected with the monoclonal antibody in PRMT2^{+/+} extracts, in contrast to PRMT2^{-/-} MEF extracts (FIG. 13C). The presence of RB was observed in immunoprecipitates of endogenous PRMT2 from PRMT2^{+/+} MEFs (FIG. 13D, lane 2) but not PRMT2^{-/-} MEF extracts (FIG. 13D, lane 3). Immunoprecipitation using a control mouse monoclonal anti-Flag antibody did not co-immunoprecipitate RB in PRMT2^{+/+} MEFs (FIG. 13D, lane 4). RB protein was not differentially expressed in PRMT2^{+/+} and PRMT2^{-/-} mice (FIG. 13E). These findings demonstrate an endogenous interaction between PRMT2 and RB in vivo.

Increased Endogenous E2F Activity and an Earlier S Phase Entry in PRMT2^{-/-} Cells

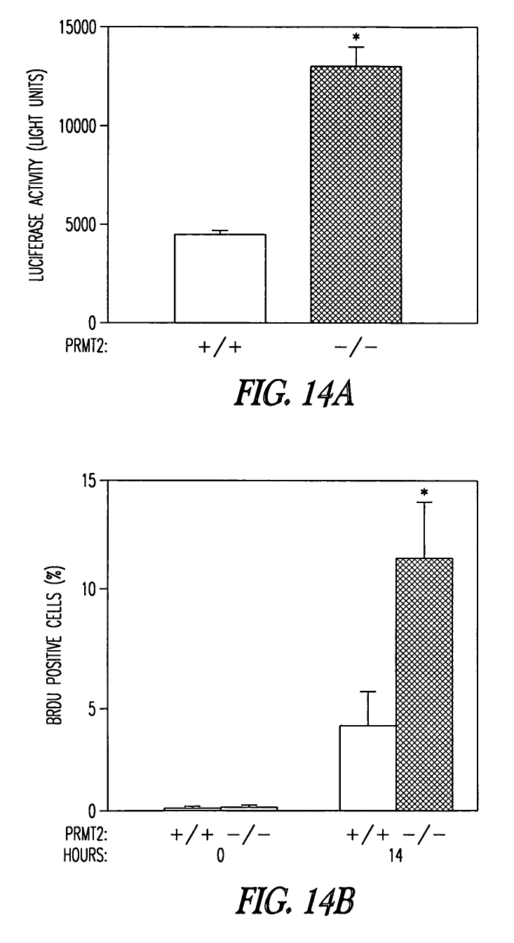

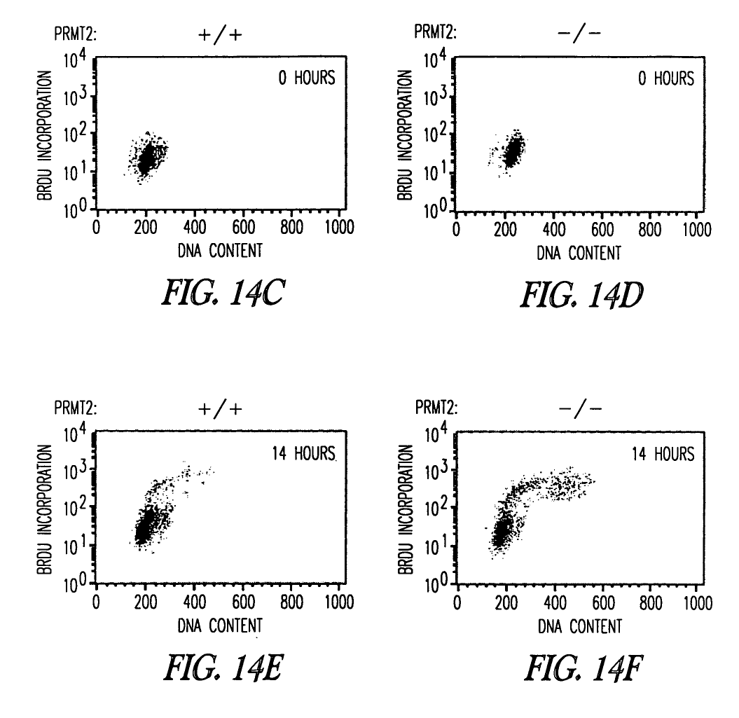

To determine whether PRMT2 directly regulates E2F transcription, asynchronously growing PRMT2^{+/+} and PRMT2^{-/-} MEFs were transfected with a luciferase reporter construct, driven by an adenovirus EIB TATA box flanking with four tandem E2F consensus sites (E2F4B-Luc). E2F reporter activity in PRMT2^{-/-} MEFs was approximately three-fold higher in contrast to PRMT2^{+/+} MEFs (FIG. 14A), suggesting that PRMT2 suppresses E2F transcriptional activity.

The family of E2F transcription factors regulates G₁/S transition, and its activity, in turn, is controlled by members of the Rb family. See, Dyson, Genes Dev. 12: 2245-62 (1998); Harbour et al. Genes Dev. 14: 2393-2409 (2000). Loss of Rb function leads to a shortened G₁ period and early S phase entry. Herrera et al. Mol. Cell Biol. 16: 2402-7 (1996). Accordingly, because the above findings indicate that PRMT2 regulates E2F activity through its interaction with RB, the functional significance of PRMT2 regulation of E2F activity in PRMT2^{+/+} and PRMT2^{-/-} cells was investigated. PRMT2^{+/+} and PRMT2^{-/-} MEFs were synchronized by serum starvation and then stimulated to enter the cell cycle by the addition of serum. S phase entry was monitored by measuring incorporation of BrdU into DNA. An approximate three fold higher percentage of BrdU positive cells was observed in PRMT2^{-/-} MEFs in contrast to PRMT2^{+/+} MEFs 14 hours after serum release (FIGS. 14B-F), indicating an earlier S phase entry in PRMT2^{-/-} MEFs compared with PRMT2^{+/+} MEFs. These data suggest that endogenous PRMT2 plays a negative regulatory role in G₁ to S phase transition.

Thus, as shown in this Example, PRMT2, a member of protein arginine methyltransferase family, interacts with RB through its AdoMet binding domain. This interaction is specific for PRMT2, since other PRMT family members do not bind RB. PRMT2 forms a complex with RB and E2F and represses E2F activity in a RB-dependent manner. An endogenous interaction between PRMT2 and RB is demonstrated in vivo. Deletion of PRMT2 in mice leads to a loss of RB interaction and activation of E2F transcription, indicating that PRMT2 directly regulates endogenous E2F activity and cell cycle progression

PRMTs modulate transcription through histone and/or co-factor methylation. As illustrated herein, the methyltransferase activity of PRMT2 is not required for repression of E2F activity. As shown herein, PRMT2 plays diverse roles in transcriptional regulation through different mechanisms that depend on its binding partner. The present study indicates that RB is indispensable for the E2F repression by PRMT2, suggesting that PRMT2 may recruit other co-repressors, or may affect the function of other co-repressor in RB complex.

The direct role of endogenous PRMT2 in the regulation of E2F activity was demonstrated herein by gene targeting of PRMT2. Endogenous PRMT2-RB interaction was detected in PRMT2^{+/+} MEFs but not in PRMT2^{-/-} MEFs. Increased E2F activity in PRMT2^{-/-} MEFs was shown by a reporter assay using a synthetic promoter specifically driven by E2F. In addition, PRMT2^{-/-} MEFs displayed earlier S phase entry than PRMT2^{+/+} MEFs following serum exposure. These findings indicate that PRMT2 deletion leads to impaired RB function and that PRMT2 is an important co-factor for RB function. However, PRMT2^{-/-} mice are viable and reproduce. Because there are many RB binding proteins which negatively regulate E2F function, the loss of PRMT2 protein might be compensated by other RB co-factors.

In conclusion, the results provided herein represent a novel mechanism by which E2F activity is regulated by the protein arginine methyltransferase, PRMT2, through its interaction with RB. The results also support the concept of diverse transcriptional regulation by PRMT family members through several mechanisms, including histone methylation, transcription factor methylation, and RNA splicing.

### EXAMPLE 3

### PRMT-2 Regulates Glucose and Lipid Metabolism

PRMT-2 knockout mice were generated as described above in Example 2. These mice gained less weight, had reduced food intake and a marked decrease of glycogen storage in the liver. Leanness in PRMT2^{-/-} mice was accompanied by lower concentration of circulating leptin as well as lower concentrations of blood glucose, serum insulin and triglycerides. Resistance for food-dependent obesity in PRMT2^{-/-} mice was also indicated by lesser weight gain and lower accumulation of body fat on a high-fat feeding. After intraperitoneal administration of leptin, PRMT2^{-/-} mice lost their weight and reduced food intake more than wild-type littermates did. In situ hybridization revealed that both PRMT2 and Signal Transducers and Activators of Transcription 3 (Stat3) mRNA were coexpressed in the hypothalamus including the arcuate, ventromedial hypothalamic and paraventricular hypothalamic nuclei. PRMT2 directly bound Stat3 and methylated arginine31 residue of Stat3 through its AdoMet domain in vivo and in vitro. Absence of PRMT2 resulted in decreased methylation and a prolonged tyrosine phosphorylation of Stat3. mRNA expression of hypothalamic proopiomelanocortin was significantly increased in leptin-treated PRMT2^{-/-} mice in comparison with leptin-treated wild-type controls. These results indicate that PRMT2 has a pivotal role in weight control through modulation of leptin-Stat3-melanocortin signaling. PRMT-2 is therefore a new target in the treatment of several metabolic disorders, such as type 2 diabetes mellitus, food dependent obesity and hyperlipidemia.

### Material and Methods

Establishment of PRMT-2^{-/-} Mice.

As described in the previous Example and shown in FIG. 12, the PRMT-2 targeting vector was linearized and electroporated into D3 ES cells. Clones doubly resistant to G418 (300 g/ml) and Gancyclovir (0.5 g/ml) were tested for homologous recombination by Southern blot analysis. DNA from ES cells was digested with EcoRI and a genomic probe (5′ probe: nt. 84051-85095 from AC006507) was used for Southern hybridization to confirm homologous recombination. Two ES cell clones were used to produce chimeras with >90% agouti coats.

Male chimeras from both clones produced F1 agouti animals, 50% of which were F1 heterozygotes. Male and female F1 heterozygotes identified by Southern blot analysis were interbred to produce F2 progeny. A genomic PCR assay was then used for subsequent genotyping using a primer common to both genotypes (primer b), having the sequence 5′-CTGAGGTATTACCAGCAGA CA-3′ (SEQ ID NO:33), the wild type allele specific primer (primer a) 5′-CTCTCTGATGCAGGTCTAC-3′ (SEQ ID NO:34), and the mutant allele specific primer (primer c) 5′-CCGGTGGATGTGGAATGTGT-3′ (SEQ ID NO:35). All animals undergoing experimental procedures were individually genotyped to ascertain which PRMT-2 genotype they had by PCR.

All mice were housed in a temperature-, humidity-, and light-controlled room (14 hours light/10 hours dark cycle) with free access to water and standard rat diet (352 kcal/100 g), except for the experiment involving high-fat feeding. Male mice were used in the studies reported here except for the phenotypic data reported for female mice. Animal care and all experimental protocols were reviewed and approved by Animal Care Use Committee of National Heart, Lung and Blood Institute and conducted in accordance with the guidelines of National Institutes of Health.

Body Weight, Snout-Anus Length and Food Intake Measurements.

Body weight was measured weekly, beginning at 6 weeks of age. Snout-anus length was measured with a micrometer on 12-week-old anaesthetized animals. Food intake was measured daily for 14 days in 12-13-week-old mice.

High-Fat Feeding.

Mice were housed three to four mice per hanging cage with food and water available ad libitum. The high-fat diet was a modification of the AIN-93G formula with added lard in a paste form (Bio-Serv, Frenchtown, N.J.) and consisted of 25% carbohydrate, 21% protein and 54% fat content as a percentage of caloric content. Wild-type and PRMT^{-/-} mice were fed the high-fat diet for a period of 10 weeks. Body weight was measured once a week. For body composition analysis, epidermal, inguinal, subcutaneous and interscapular fad pad masses were dissected and measured at the end of the high-fat feeding schedule.

Blood Glucose and Serum Insulin, Triglyceride, Leptin Measurements.

Whole blood was obtained from the tail vein of fasting or fed mice. Blood glucose was assessed by an automatic glucometer (Roche Diagnostic Corp., Indianapolis, Ind.). Serum was taken from the heart of fasting mice at 10:00-11:00 A.M. Serum insulin concentrations were measured by ELISA using rat insulin as a standard (Amersham Pharmacia Biotech, Buckinghamshire, UK). Serum triglycerides levels were determined by optimized enzyme colorimetric assay (Roche Diagnostic Corp., Indianapolis, Ind.). Serum leptin concentrations were also measured by ELISA using mouse leptin as a standard (Crystal Chem, Inc., Chicago, Ill.).

Histology.

Sections (5 μm thick) from Bousin's fixed paraffin-embedded specimens were stained with hematoxylin and eosin, and periodic acid Schiff (PAS), and examined by light microscopy.

Glucose and Insulin Tolerance Tests.

Glucose tolerance tests were performed after overnight fasting by administrating 1.5 g/kg body weight D-glucose via the peritoneal cavity, and blood samples were obtained from tail vein at 0, 15, 30, 60, 90 and 120 min after injection. For insulin tolerance tests, mice starved overnight were injected intraperitoneally with 0.5 units/kg body weigh human regular insulin (Novolin R; Novo Nordisk, Copenhagen), and blood were sampled from the tailed at 0, 15, 30 and 60 min after injection. Blood glucose values were determined from whole venous blood taken by using an automatic glucose monitor previously described.

In Vivo Insulin Stimulation.

After overnight fasting, 8-week old mice were anesthetized with Ketamine, the cervical portion of the anesthetized mice was opened, the right jugular vein was exposed and 300 mg gastrocnemius muscle from one hind limb was rapidly removed. The gastrocnemius muscle was immediately frozen in liquid nitrogen and 5 units human regular insulin was injected into the inferior vena cava. The muscle from the other hind limb was removed at 5 min and instantly frozen in liquid nitrogen. Frozen samples were powdered and homogenized in the buffer containing 25 mM Tris-HCl (pH 7.4), 10 mM Na₃VO₄, 100 mM NaF, 50 mM Na₄P₂O₇, 10 mM EGTA, 10 mM EDTA, a protease inhibitor cocktail tablet, Complete (Boehringer Mannheim), and 1% (vol/vol) Nodiet P-40. Homogenates were incubated at 4° C. for 1 hr to solubilize proteins. The samples were then centrifuged at 55,000 rpm for 1 hr at 4° C. and the supernatants were used for immunoprecipitation and immunoblot analysis of insulin receptor substrate-1 (IRS-1).

Leptin Sensitivity Study.

To determine leptin sensitivity, 9- to 13-week-old mice, matched for similar body weight at both day 4 and day 0, were individually caged and body weight and food intake were measured once daily (5:30 pm) throughout the experiment. For the first 7 days, mice were injected intraperitoneally twice daily (12:00 pm and 6:00 pm) with PBS to establish a baseline of weight change and food intake. Afterwards, PBS was replaced with recombinant mouse leptin (Sigma-Aldrich Inc.) at 0.1 mg/kg during the 6 consecutive days. Body weight and food intake was measured once a day during the experiment.

Antibodies.

Rabbit polyclonal anti-IRS-1 antibody and goat polyclonal anti-phospho-IRS-1 antibody were purchased from Santa-Cruz Biotechnology (Santa Cruz Biotechnology, Santa Cruz, Calif.). Rabbit anti-STAT3 antibodies and rabbit polyclonal anti-phospho STAT3 [pStat3 (pY705)] antibodies were purchased from Cell Signaling Technology (Beverly, Mass.). Mouse monoclonal anti-phosphotyrosine antibody (4G10) was purchased from Upstate Biotechnology (Lake Placid, N.Y.). Mouse monoclonal anti-arginine (mono- and di-methyl) antibody (ab412 or α-metR) was purchased from Abcam, Inc. (Cambridge, Mass.). Rabbit polyclonal and mouse monoclonal anti-Flag antibodies were purchased from Sigma (St. Louis, Mo.). Rabbit polyclonal anti-PRMT-2 and mouse monoclonal PRMT-2 antibody were purchased from Biocarta (San Diego, Calif.) and A & G Pharmaceutical, Inc. (Columbia, Md.), respectively.

Plasmids.

Mouse PRMT-2 cDNA was cloned by RT-PCR using total RNA extracted from mouse cardiac tissues. The following oligonucleotide pairs were used for PCR: 5′-AAGGATCCAGCCCCAGTTATGAGACATGAT-3′ (SEQ ID NO:36) and 5′-AAAAGCTTCTTCTTTCACTGAGATGCATGC-3′ (SEQ ID NO:37) and pVR1102 were used as a plasmid for subcloning. Mouse PRMT-2 motif 1 mutant was generated from the wild-type pVR1012 PRMT-2 construct using QuikChange XL Site-Directed Mutagenesis Kit (Stratagene, La Jolla, Calif.). The plasmid encoding mouse STAT3 pcDNA3 was a kind gift from Dr. J. E. Darnel (The Rockefeller University, New York, N.Y.).

Plasmid encoding GST fusion proteins of STAT3 was generated by PCR from a mouse STAT3 pcDNA template using the following oligonucleotides: 5′-GGCGAATTCACTGCAGCAGGATGGCTCAGTG-3′ (SEQ ID NO:38) and 5′-GCTGTCGACTTGTGGTTGGCCTGGCCCCCTTG-3′ (SEQ ID NO:39). The resulting PCR product was cloned into EcoRI and SalI sites of pGEX6P-3 (Amersham Biosciences). The same region with the Arg31→Ala mutant was generated from the wild-type pGEX6P-3 STAT3 construct using the above Mutagenesis Kit. GST fusion proteins were expressed in BL21 (DE3) cells and extracts were prepared as described previously. Smith, D. B. & Johnson, K. S. Gene 67, 31-40 (1988).

Bacterial Expression and Purification of Wild-Type and Mutated Stat3.

Expression of the glutathione S-transferase (GST)-Stat3 fusion proteins were prepared according the manufacturer's protocol (Amersham Pharmacia Biotech, Inc., Piscataway, N.J.), but with the following modifications. An overnight culture of Top10 One Shot Competent cells containing the pGEX6P3 wild-type or mutated Stat3 plasmid grown in Luria broth supplemented with carbenicillin (100 μg/ml) was diluted 1:15 into 400 ml of the same medium and grown until OD650 0.5 at 22° C. followed by another culture until OD650>2.0 in the presence of 0.1 mM isopropyl-β-D-thiogalactopyranoside (Sigma-Aldrich Inc.). The cells were then pelleted, resuspended in 20 ml of ice cold phosphate-buffered saline (137 mM NaCl, 8.10 mM Na₂HPO₄, 2.68 mM KCl, 1.47 mM KH₂PO₄) containing 1% Triton-X 100, and sonicated. Wild-type and mutated GST-Stat3 were bound to glutathione sepharose 4B (Amersham Biosciences), and after extensive washing eluted in elution buffer (50 mM Tris-HCl, pH 9.6, 10 mM glutathione, reduced form). The fusion proteins were dialyzed by PBS using Slide-A-Lyzer 10K Dialysis Cassettes (Pierce, Rockford, Ill.) and stored at −80° C.

Cell Culture and Transient Transfection.

Vascular smooth muscle cells (VSMC) were prepared from the thoracic aorta of 12-week-old-male wild-type and PRMT-2^{-/-} mice by the explant method. Mouse embryo fibroblasts (MEFs) were prepared from 13.5-day wild-type and PRMT-2^{-/-} embryos. VSMCs and MEFs were cultured in Dulbecco's modified Eagle's medium (DMEM) containing 10% fetal calf serum (FCS) at 37° C. in a humidified atmosphere of 95% air-5% CO₂. The integrity of PRMT-2 expression in the established VSMCs and MEFs lines was confirmed by Western and immunofluorescence analysis (data not shown). Quiescent VSMC (3-5th passages) that had been serum-starved for 48 hr were used in the following experiments. HEK293 cells were also grown in DMEM plus 10% FCS and were transiently transfected with FuGENE6 transfection reagent (Roche Applied Science, Indianapolis, Ind.) according to the manufacture's protocol. For each transfection, 2 μg of expression construct for mouse PRMT-2 and 8 μg for mouse STAT3 were used. After 24-48 hr of transfection, cells were used for the following experiments.

Immunoprecipitation and Immunoblotting.

Immunoprecipitation of STAT3 was performed as follows. Cells treated with or without 100 nM mouse leptin for selected times were washed with phosphate buffered saline and lysed in Nonidet P-40 (NP-40) buffer (20 mM Tris-HCl, pH 8.0, 150 mM NaCl, 25 mM EDTA, 1.0% Triton-X, 0.1% SDS, 10% glycerol, 100 mM NaF, 100 mM Na₃P₂O₇, 1.0% deoxycholic acid, 1 mM Na₃VO₄, 1× protease inhibitors cocktail; Roche Diagnostic Corp., Indianapolis, Ind.) or RIPA buffer (50 mM Tris-HCl, pH 8.0, 150 mM NaCl, 1% NP-40, 0.5% Na-deoxycholate, 0.1% SDS, 1× protease inhibitors cocktail). After the lysates were incubated to solubilize proteins for 30 min at 4° C., and before they were centrifuged at 15,000 rpm for 10 min at 4° C., the supernatants were rocked with either polyclonal anti-STAT3 antibodies (1:2,000) or monoclonal anti-Flag antibodies for 14 hr at 4° C., and then with protein G agarose for 3 hr at 4° C. For in vitro stimulation, the supernatants from extracted tissue homogenates were rocked with 2 μg polyclonal anti-IRS-1 antibody, and then protein G agarose for 3 hr at 4° C. The beads were washed three times with lysis buffer without proteinase inhibitors, solubilized in 15 μl 3×SDS-polyacrylamide gel electrophoresis (PAGE) buffer (187.5 mM Tris-HCl, 6% SDS, 30% glycerol, 150 mM dithiothreitol, 0.3% bromophenol blue, pH6.8), and subjected to the immunoblotting.

Cytoplasmic/nuclear extracts were prepared by Dounce homogenizing cells in Cytoplasmic lysis buffer (20 mM HEPES, pH7.9, 10 mM KCl, 1 mM MgCl₂, 1% NP-40, 10% glycerol, 100 mM NaF, 1 mM Na₃VO₄, 1× protease inhibitors cocktail), and sedimenting nuclei by centrifugation at 1,000 rpm for 5 min. The supernatants were removed as samples of cytoplasmic fraction. The nuclei were extracted with nuclear lysis buffer (20 mM HEPES, pH7.9, 300 mM NaCl, 10 mM KCl, 1 mM MgCl₂, 1% NP-4-, 10% glycerol, 00 mM NaF, 1 mM Na₃VO₄, 1× protease inhibitors cocktail), and then subjected to the immunoblotting.

Tissue detergent extracts were prepared by dissection and homogenization in lysis buffer (25 mM HEPES, pH 7.9, 1% NP-40, 137 mM NaCl, 1× protease inhibitors cocktail) using a Polytron homogenizer (Brinkman Instruments, Westbury, N.Y.). The samples were centrifuged at 2,000×g for 5 min at 4° C., and the resulting supernatants were then re-centrifuged at 14,000×g for 20 min at 4° C. The protein content of the final supernatant was dissolved in 3×SDS-PAGE buffer.

Immunoblotting was performed by boiling samples for 5 min at 95° C. followed by centrifugation for 1 min at 4° C. Aliquots of the supernatant were subjected to 7.5, 10% or 4-15% SDS-PAGE. Proteins in the gel were transferred to a nitrocellulose membrane by electroblotting. The membranes were treated with either anti-pTyr, anti-STAT3, anti-phospho STAT3, anti-α-metR, anti-IRS-1, anti-Flag, anti-phospho IRS-1, anti-arginine (mono- and di-methyl), or anti-β-actin antibodies followed by incubation with secondary antibodies conjugated with HRP. Immunoreactive proteins were detected either by ECL or by ECL plus system (Amersham Pharmacia Biotech). In some cases, bands were quantified using the NIH Image software (Image J version 1.2; National Institutes of Health, Bethesda, Md.). The figures are representative of 3 experiments.

Northern Blot Analysis.

Total RNA from skeletal muscle was extracted using an acid guanidinium thiocyanate-phenol-chloroform method subjected to Northern blot analysis. Briefly, tissue RNAs (1 μg) were separated by formaldehyde-1.1% agarose gel electrophoresis and transferred to a MagnaGraph nylon membrane (Micron Separations, Westborough, Mass.). After UV wave cross-linking, RNA immobilized on the membrane was hybridized with mouse STAT3 and PRMT-2 cDNA probes in the presence of 50% formamide at 42° C. The probes were labeled with 50 μCi of [α-³²P]deoxy-CTP triphosphate (Amersham Biosciences) by the random primed labeling method using Rediprime II (Amersham Biosciences). The membrane was washed, with the final wash being 0.1×SSPE (15 mM NaCl, 1 mM NaH₂PO₄, and 0.1 mM EDTA)-0.5% SDS at 50° C. The washed blot was subjected to autoradiography with an intensifying screen for 24 hr.

In some cases, a mouse MTN Blot was purchased from BD Biosciences Clontech (Palo Alto, Calif.). Mouse PRMT2 and Stat3 cDNAs, and human β-Actin cDNA (BD Biosciences Clontech) probes were labeled with 50 μCi of [α-³²P]deoxy-CTP triphosphate (Amersham Biosciences) by the random primed labeling method using Rediprime II (Amersham Biosciences) and unincorporated label was separated using Quick Spin (G-50 Sephadex) (Roche Diagnostic Corp.). The membrane was hybridized with the probe in the presence of PerfectHYB plus (Sigma) at 68° C. for 18 hr and was washed finally in 0.1×SSC (15 mM NaCl, 1.5 mM C₆H₅O₇ Na₃.2H₂O)-0.1% SDS at 50° C. After washing, the membrane was dried and was exposed to Kodak BioMax MS film (Eastman Kodak Company, Rochester, N.Y.) with Intensifying Screen (Fisher Scientific, Pittsburgh, Pa.) for 1-3-day at −80° C.

In Situ Hybridization

Brains were removed immediately after decapitation, frozen in 2-methylbutane (Aldrich) with a bed of crushed dry ice for 6 sec. 16-μm thick coronal sections were cut on a cryostat at −20° C. and were thaw-mounted on RNase-free slides (K•D Medical Inc.). Labeled riboprobe was generated by in vitro transcription reaction using linearization of cDNA template and 2 μCi of [³⁵S]UTP (Amersham Biosciences), and unincorporated label was separated using Autoseq (G-50) column (Amersham Biosciences). After pretreatment with 0.25% acetic anhydride, sections were hybridized with sense and antisense riboprobes corresponding to nucleotides 295-798 of mouse PRMT2 cDNA or to the nucleotides 883-1398 of mouse Stat3 cDNA in the presence of hybridization buffer (20 mM Tris-HCl, pH7.5, 50% Formamide, 300 mM NaCl, 1.0 mM EDTA, 1× Denhard's solution, 10% dextran sulfate, 150 mM DTT, 0.2% SDS) at 54° C. After incubation for 18 hr, the sections were washed finally in 0.1×SSC (15 mM NaCl, 1 mM NaH₂PO₄, and 0.1 mM EDTA) containing 2 mM DTT at 22° C., were dried, and were subjected to autoradiography with film for 5-day at room temperature.

In Vitro Methylation Assay.

293 cells were transiently transfected with expression vectors encoding Flag-tagged wild-type or mutant mouse PRMT2. After incubation for 48 hr, cells were lysed with 500 μl of RIPA buffer and 1.8 mg of 293 cell lysates was incubated with 40 μl of Anti-Flag M2 Affinity Gel (Sigma) for 3 hr at 4° C. Immune complexes were washed three times with lysis buffer without proteinase inhibitor and subjected to in vitro methylation reaction. Mouse fibroblasts were grown to 80% confluence on a 10 cm plate. Cells were washed and scraped off the plate into 500 μl of PBS (pH 7.4), and were lysed by sonication. After centrifugation at 15,000 rpm for 10 min at 4° C., the supernatants were used as the enzyme source. In vitro methylation reactions were performed by adding the immune complexes or cell lysates to 0.64 μg Histone and/or 1 μg of GST, GST-STAT3 or GST-STAT3 Arg31→Ala31 using 2 μCi of the methyl donor S-adenosyl-1[methyl-3H]methionine ([3H]-AdoMet) (Amersham Biosciences) in a final volume of 35 μl. The reactions were incubated for 1 hr at 4° C. and were terminated by addition of 3×SDS-loading buffer. The samples were subjected to SDS-PAGE in 4-15% Tris-HCl gradient gel (Bio-Rad Laboratories, Inc., Hercules, Calif.), transferred to a poly(vinylidene difluoride) (PVDF) membrane, sprayed with En³hance (Perkin-Elmer Life and Analytical Sciences, Boston, Mass.), and exposed to Kodak BioMax MS film (Eastman Kodak Company, Rochester, N.Y.) with Transcreen LE Intensifying Screen (Eastman Kodak Company) for 7-10 days at −80° C. After autoradiography, the membrane was washed twice with the same buffer used for protein transfer and then stained with Coomassie brilliant blue for 5 min to detect GST protein amounts.

Immunocytochemistry.

VSMCs were plated on four-chamber glass Lab-Tek (Nunc Inc., Naperville, Ill.) slides. After 48 hr of serum-starvation, quiescent cells were stimulated with or without mouse leptin for selected times. Control cells received no leptin. Stimulation was terminated by removal of medium and cells were washed three times with ice-cold PBS. The cells were fixed in 4% paraformaldehyde for 15 min at room temperature. After washing with ice-cold PBS, the cells were immersed in PBS containing 0.2% Triton X-100 for 5 min at room temperature, and treated with PBS containing 3% BSA for 1 hr at room temperature to block non-specific antibody binding. The cells were then incubated with phospho-STAT3 antibody in Tris-buffered saline containing 1% BSA overnight at 4° C. After overnight incubation, the cells were washed four times with ice-cold PBS and incubated with an FITC conjugated anti-rabbit IgG for 1 hr at room temperature. The incubation was terminated with aspiration of the secondary antibody, and the chambers were removed from the slides. After washing with ice-cold PBS and H₂O, the slides were mounted with Vectashield mounting medium containing diamidophenolindole (Vector Laboratories, Inc., Burlingame, Calif.) for nuclear staining. Results were visualized on a fluorescence microscope (Nikon Eclipse E800, Nikon, Tokyo, Japan) and pictures were taken with a digital camera (Retiga 1300, QImaging, Burnaby, Canada).

Immunohistochemistry.

After overnight fasting, brains of wild-type and PRMT2−/− mice treated with or without mouse leptin (1.67 μg/g body weight) for 90 min were removed immediately after decapitation, frozen in 2-methylbutane with a bed of crushed dry ice for 6 sec. 16-μm thick coronal sections were cut on a cryostat at −20° C. and were thaw-mounted on slides (K•D Medical Inc., Columbia, Md.). Sections were fixed with acetone for 10 min at 4° C. and then incubated in methanol for 30 min at −20° C. After treatment with 0.3% H₂O₂ in methanol for 30 min at room temperature to block endogenous peroxidase, sections were blocked in Tris-buffered saline (TBS)-Ca (100 mM Tris-HCl, pH 7.4, 150 mM NaCl, 1 mM CaCl₂) containing 10% normal goat serum and 3% bovine serum albumin (BSA) and incubated with a rabbit anti-pStat3 (pY705) diluted in the same blocking solution as previously described above overnight at 4° C. On the next day sections were washed three time with TBS-Ca, incubated with a biotinylated secondary goat antibody in TBS-Ca containing 1% BSA for 1 hr at room temperature and then treated with ABC solution (Vector laboratories, Inc., Burlingame, Calif.) for 1 hr. In a final step, the signal was developed by DAB solution (Vector laboratories), giving a brown precipitate, dehydrated and mounted using Permount (Sigma-Aldrich). Pictures were taken as described above with a digital camera and a brightfield microscope (Nikon Eclipse E600, Nikon, Tokyo, Japan).

Real-Time Reverse Transcription PCR.

Mice in the 8-12 week of age allowed free access to chow and water were intraperitoneally injection for 3 days with either PBS or recombinant mouse leptin (Sigma) at a dose of 0.5 μg/g body weight twice a daily (9:00 am and 7:00 pm). Hypothalamus was isolated from the mice at 6 hr after leptin treatment and snap-frozen. Total hypothalamic RNA was isolated using Trizol (Invitrogen) according to the manufacturer's instructions. Total RNA was treated with ribonuclease-free deoxyribonuclease (DNase) I for 30 min using a commercially available kit (Invitrogen) to eliminate contamination of genomic DNA. The RNA samples were reverse transcribed with TaqMan Reverse Transcription Reagents (Applied Biosystems, Foster City, Calif.) and subjected to automated fluorescent RT-PCR on an ABI PRISM 7700 Sequence Detection System (Applied Biosystems). All TaqMan probes were labeled with the reporter fluorescein at the 5′-end and with the quencher tetramethylrhodamine at the 3′-end. The following oligonucleotides derived form the mouse POMC and NPY were synthesized and used as primers and probes for Real-time PCR: POMC (sense, 5′-CTGCTTCAGACCTCCATAGATGTG-3′ (SEQ ID NO:42), antisense, 5′-CAGCGAGAGGTCGAGTTTGC-3′ (SEQ ID NO:43), probe, 5′-6FAM-CAACCTGCTGGCTTGCATCCGG-TAMRA-3′, SEQ ID NO:44); NPY (sense, 5′-TCAGACCTCTTAATGAAGGAAAGCA-3′ (SEQ ID NO:45), antisense, 5′-GAGAACAAGTTTCATTTCCCATCA-3′ (SEQ ID NO:46) probes, 5′-6FAM-CCAGAACAAGGCTTGAAGACCCTTCCAT-TAMRA-3′ (SEQ ID NO:47)). TaqMan Rodent GAPDH Control Reagents (Applied Biosystems) were used as a control primers and probe for each template. Each predicted RT-PCR product spanned an intron/exon junction. The reactions were incubated for 2 min at 50° C., followed by 10 min at 95° C., and then 40 cycles of 15 sec at 95° C. and 1 min at 60° C. Each RT-PCR reaction was performed in triplicate in a final volume of 20 μl and assessed by the comparative Ct (ΔΔCt) method (Applied Biosystems, ABI Prism 7700 Users Bulletin #2).

Statistical Analysis.

Data are presented as means±standard error of the mean (SEM). Comparisons between experimental groups were performed using unpaired Student's t test or ANOVA when appropriate. The difference was considered to be significant if p<0.05.

### Results

Generation of PRMT-2-Deficient Mice.

To assess the physiological function of PRMT-2, a PRMT-2 knockout strain of mice was generated. The targeting vector employed replaced 4.2 kb of PRMT-2 genomic sequence, including the ATG-coding exon, with a neoR cassette oriented in the opposite direction as the endogenous PRMT-2 gene (FIG. 15A). The deleted coding region included a 96 amino acid three helix segment that constitutes part of the AdoMet domain, and also forms part of the Rossmann fold.

To generate the PRMT-2 knockout strain of mice the targeting vector was electroporated into J1ES cells, and G418-resistant clones were screened by PCR to detect homologous recombinants. Analysis of neomycin-resistant ES cell clones by Southern blotting and PCR with PRMT-2 specific probe showed that the mutation involved a single vector integration site (FIGS. 15B and 15C). Two independent cell clone isolates were injected into C57BL6J blastocysts to generate chimeric mice. These chimeras were used to establish two independent lines of PRMT-2-deficient mice (Chimera #31 and #8) with >90% agouti coats. Mice from both two lines displayed the same phenotype, which is described in detail below.

Heterozygous mice from each of these lines were intercrossed, and their offspring were genotyped by PCR and Southern blotting (FIGS. 15B and 1C). All three genotypes (PRMT-2^{+/+}, PRMT-2^{+/-} and PRMT-2^{-/-}) were obtained at the expected 1:2:1 Mendelian frequency (54:92:56). PRMT-2^{-/-} mice could be maintained for at least 1.5 year without apparent gross abnormalities and were fertile.

The relative expression levels of PRMT-2 mRNA and protein were determined by Northern analysis (FIG. 15D) and immunoblotting (FIG. 15E) of various mouse tissues. More abundant sites of PRMT-2 expression were present in brain and skeletal muscle tissues, followed by heart, lung and spleen. Absence of PRMT-2 expression was verified by immunoblotting of tissue extracts from PRMT-2^{-/-} mice (FIG. 15F). Interestingly, STAT3 protein was ubiquitously expressed, whereas higher expression was also observed in brain and skeletal muscle, suggesting that these tissues are possible targets for associable effect of PRMT-2 with STAT3 (FIGS. 15D and E).

PRMT-2 Deficient Mice are Lean and Show Lower Food Intake.

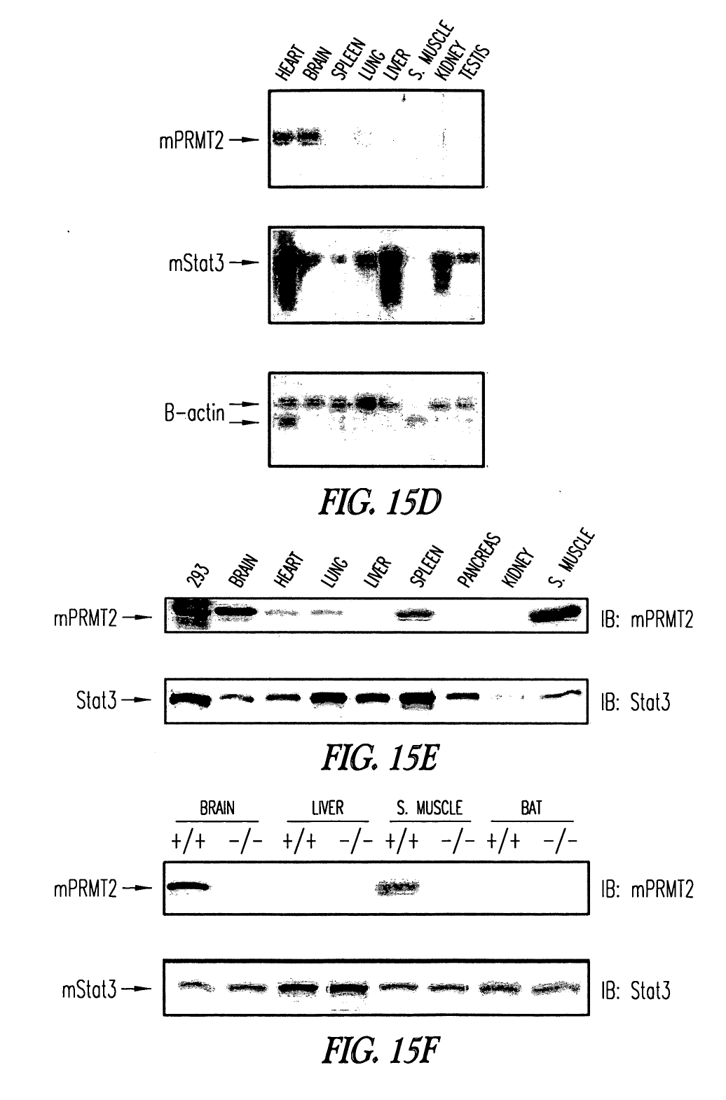

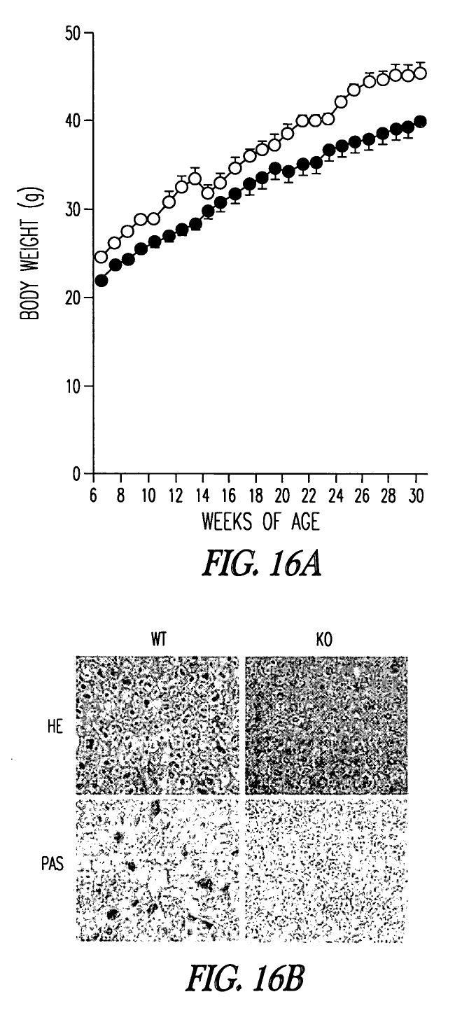

PRMT-2^{-/-} male mice gained less weight than age-matched control wild-type mice (FIG. 16A). This difference first became significant at 6 weeks. As shown in Table 1, the average weight of male PRMT-2^{-/-} mice was 14% less than controls by 25 weeks (P=0.001). Heterozygous (PRMT-2^{+/-}) males showed an intermediate weight gain between wild-type mice and PRMT^{-/-} mice on this diet (Table 1), suggesting that PRMT-2 affected the reduction of their weight in a gene dosage-dependent manner. While there were no differences between weights of female wild-type and PRMT2^{-/-} mice at 12-weeks (P=0.8615), PRMT2^{-/-} females were lean at 30 weeks of age compared with wild-type mice (P=0.024) (Table 1). An analysis of liner growth by measurement of snout-anus length in 12 weeks of age revealed that male and female PRMT2^{-/-} mice were about 4% and 3% shorter than male and female wild-type mice, respectively (P=0.045 and 0.003) (Table 1), indicating that PRMT2 may also participate in linear growth.

Leanness of PRMT2^{-/-} mice could result from decreased food intake, increased energy expenditure and/or malabsorption. To address whether decreased food intake is responsible for the lesser weight gain, food intake of wild-type and PRMT2−/− mice were monitored over 14 days. The average food intake was significantly decreased in both male and female PRMT2^{-/-} mice compared with age-matched wild-type mice (P=0.017 and 0.005, respectively) (Table 1), indicating that a decreased food intake in PRMT2^{-/-} mice, at least, partially contributes to their lesser weight gain. PRMT2^{-/-} mice did not show any gross changes in their stools compared with wild-type mice (data not shown), suggesting that malabsorption, in particular lipid malabsorption, was unlikely to be the cause of leanness in PRMT2^{-/-} mice.

TABLE 1
Phenotypic data for wild-type, heterozygote and PRMT2^{-/-} mice^{a}

| | Male | | | Female | |
|---|---|---|---|---|---|
| Genotype | +/+ | +/− | −/− | +/+ | −/− |
| Body wt. at | 32.4 ± 0.9 | 30.8 ± 0.7** | 27.8 ± 0.7** | 23.0 ± 0.8 | 22.8 ± 0.7 |
| 12-wks (g) | | | | | |
| Body wt. at | 45.2 ± 1.3 | 42.7 ± 0.7* | 39.8 ± 0.4** | 29.8 ± 1.2 | 28.2 ± 0.9* |
| 30-wks (g) | | | | | |
| Snout-anus | 10.6 ± 0.1 | 10.3 ± 0.1* | 10.3 ± 0.1* | 10.0 ± 0.1 | 9.72 ± 0.07** |
| length (mm) | | | | | |
| Feeding (g | 62.3 ± 4.4 | (ND) | 50.6 ± 2.8* | 52.6 ± 1.9 | 42.8 ± 1.2* |
| per 14 days) | | | | | |
| Fasting | 106.0 ± 3.3 | 97.1 ± 2.5 | 91.1 ± 3.9* | 106.7 ± 2.0 | 92.8 ± 3.7* |
| glucose | | | | | |
| (mg/dl) | | | | | |
| Fed-state | 156.4 ± 3.9 | 144.9 ± 4.5* | 141.7 ± 4.3* | 130.6 ± 3.5 | 127.7 ± 3.0 |
| glucose | | | | | |
| (mg/dl) | | | | | |
| Insulin | 27.8 ± 9.5 | 9.43 ± 1.07 | 8.85 ± 0.79 | 18.7 ± 4.1 | 6.95 ± 0.76* |
| (ng/ml) | | | | | |
| Triglycerides | 82.7 ± 9.3 | 55.9 ± 11.5 | 58.9 ± 7.1 | 37.6 ± 5.7 | 20.1 ± 2.2* |
| (mg/dl) | | | | | |
| Leptin | 6.67 ± 1.47 | 3.62 ± 1.01 | 2.74 ± 0.57* | 3.89 ± 0.96 | 1.24 ± 0.27* |
| (ng/ml) | | | | | |

^{a}Male or female wild-type (+/+), heterozygous (+/−) and homozygous
PRMT2^{-/-} (−/−) mice were fed a standard chew diet. Body weight at 12 and 30 weeks of age, snout-anus length and food intake at 12-13 weeks of age were measured. Blood glucose levels at fasting and fed-state, and serum insulin, triglycerides, and leptin concentrations at fasting, were determined for 8-12 weeks-old mice.
Values represent the mean ± SEM of eight mice.
*P < 005 vs. wild-type.
**P < 0.01 vs. wild-type.

Reduction of Glycogen Content in the Liver from PRMT-2 Deficient Mice.

To elucidate any histological differences between wild-type and PRMT-2^{-/-} mice, complete necropsies were performed. Hematoxylin and eosin staining of the liver revealed that cytoplasmic vacuoles were less numerous in livers from fed-state PRMT2^{-/-} mice and the hepatic cords and sinusoids were relatively more distinct than those from wild-type mice (FIG. 16B, upper two images), suggesting that glycogen content in the livers of PRMT2^{-/-} mice might be diminished at fed-state. Staining with PAS to detect glycogen deposits confirmed that livers from PRMT-2^{-/-} mice had markedly lower amounts of glycogen (FIG. 16B, lower two images). No other gross or histological abnormalities in other tissues from PRMT2^{-/-} mice including heart, thymus, spleen, pancreas, kidney, skeletal muscle of the hind limb and brown adipose tissue were observed in PRMT-2^{-/-} mice (data not shown).

Altered Glucose and Lipid Homeostasis in PRMT2^{-/-} Mice.

To assess whether the PRMT2^{-/-} genotype can affect glucose metabolism, blood glucose level and serum insulin concentration were measured at 8-12 weeks of age. Blood glucose levels of fasting and fed state male PRMT2^{-/-} mice were significant lower than those of wild-type mice (P=0.113 and 0.0179, respectively) (Table 1). A decreased concentration of fasting glucose was also observed in female PRMT2^{-/-} mice in comparison with female wild-type mice (P=0.116) (Table 1). The mean insulin concentrations of both male and female PRMT2^{-/-} mice tend to be less than those of wild type mice of the same gender (P=0.102 and 0.031, respectively) (Table 1). Serum levels of triglycerides were also reduced in both male and female PRMT2^{-/-} mice relative to wild-type mice (P-0.1115 and 0.011, respectively) (Table 1). Taken together, these results suggest that loss of PRMT2 may modulate glucose and lipid metabolism.

Resistance to Food-Dependent Obesity in PRMT-2^{-/-} Mice.

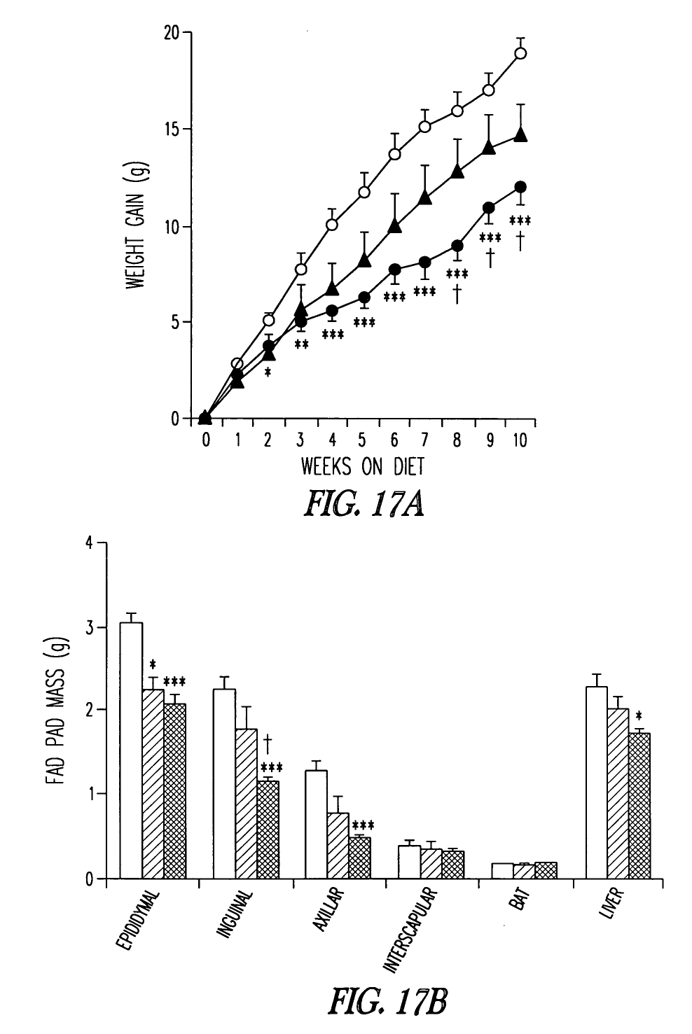

High-fat feeding induces body weight gain and obesity associated with visceral fat mass, glucose intolerance and insulin resistance in non-obese rodents (Axen et al. J. Nutr. 133:2244-49 (2003)). To determine whether PRMT^{-/-} mice show resistance to diet-induced obesity, wild-type and PRMT-2^{-/-} mice were fed a high-fat diet for a period of 10 weeks. At the end of the 10 week period, wild-type mice had significantly higher body weight than wild-type mice on a standard chow diet, however, PRMT-2^{-/-} mice were significantly more lean and weighed significantly less than both types of wild-type mice (FIG. 17A). Heterozygotic PRMT2^{+/-} mice showed an intermediate weight gain between wild-type mice and PRMT^{-/-} mice on high-fat diet (FIG. 17A). The relative leanness of PRMT-2^{-/-} mice correlated with a decrease in fat mass (FIG. 17B). Epididymal, inguinal and submaxillary fat pad weights were significantly reduced in high-fat fed PRMT2−/− mice (P=0.0006, 0.0002 and 0.0005, respectively). No differences were observed in suprascapular fat pad and brown adipose tissue (BAT) (P=0.2057 and 0.8247, respectively). The mass of the liver was also significantly decreased in PRMT2−/− mice compared with wild-type mice (P=0.0189). These data suggest that PRMT2^{-/-} mice were protected from the food-dependent obesity and increased adiposity induced by high-fat feeding.

Regulation of Leptin Signaling by PRMT-2 In Vivo.

The changes in phenotype and behavior of PRMT-2^{-/-} mice, including leanness, decreased food intake, increased insulin sensitivity and resistance to food-dependent obesity, indicate that PRMT-2 might affect leptin signaling. Therefore, serum leptin levels were evaluated in PRMT-2^{-/-} mice.

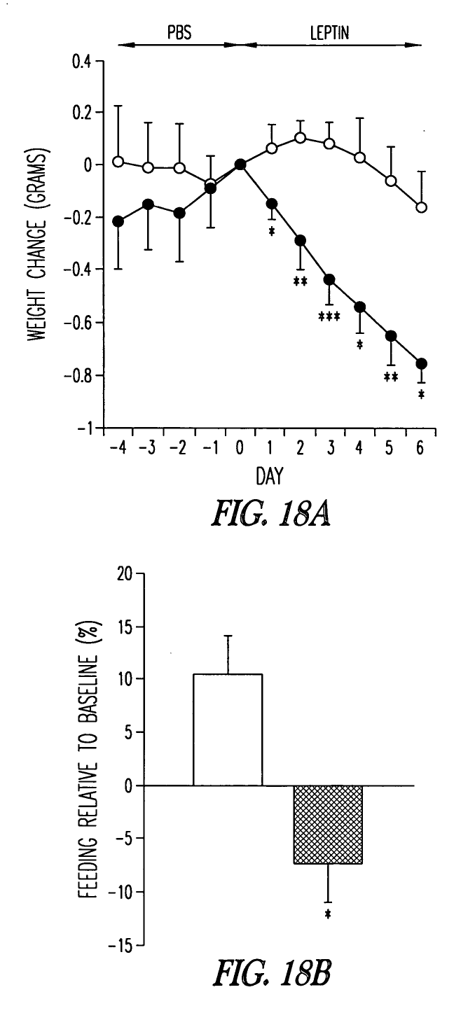

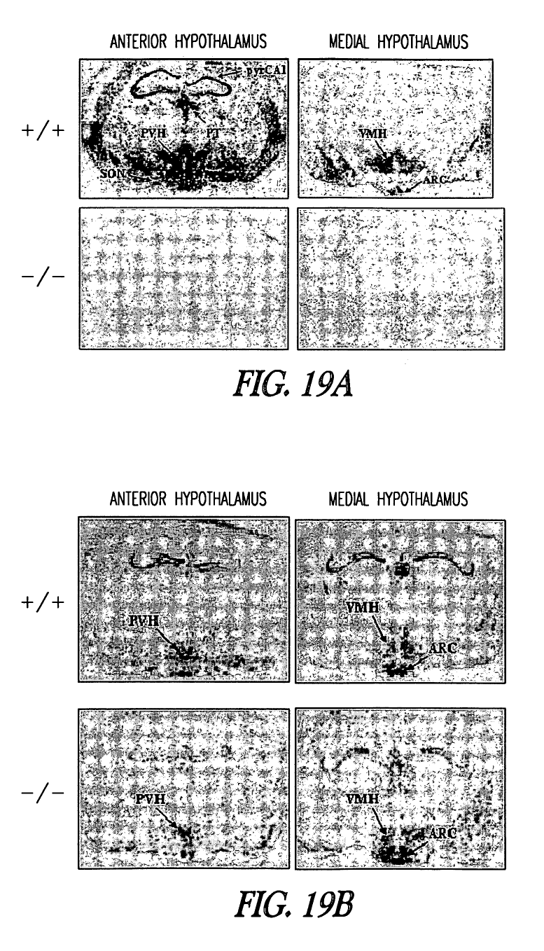

Serum concentrations of leptin in both male and female PRMT2^{-/-} mice were significantly decreased than those of wild-type mice (P=0.045 and 0.034, respectively) (Table 1), which is consistent with the hypothesis that circulating leptin levels correlate with body mass index and total body-fat mass. Next, to directly assess leptin sensitivity, the responses of wild-type and PRMT2^{-/-} mice to exogenous leptin were compared. Obesity in rodents is responsible for insensitivity to peripheral leptin injection (E1-Haschimi et al. J. Clin. Invest. 105:1827-32 (2000)). Therefore, the following types of mice, with similar levels of body weight, were selected at day-4: wild-type vs. PRMT2^{-/-} (32.10±1.32 kg vs. 32.38±2.16 kg, P=0.9245) and at day 0: wild-type vs. PRMT2^{-/-} (32.08±1.27 kg vs. 32.58±2.13 kg, P=0.8666) to avoid differences in body mass that might lead to leptin sensitivity. While it has been previously been shown that no changes of weight and feeding are observed in wild-type mice after the low-dose administration of leptin (0.1 μg/g) (Cheng et al. Dev. Cell. 2: 497-503 (2002)), the present study revealed that 4 wild-type mice began to gain some weight after peripheral injection of leptin (0.1 μg/g). However, four other wild type mice lost weight after this treatment. Consequently, as a group the eight, wild-type mice showed an only a small decrease in weight (−0.158 g for 5 days) (FIG. 18A). In contrast, all PRMT2^{-/-} mice continuously lost weight for the duration of the study (FIG. 18A). In addition, after administration of leptin, PRMT2^{-/-} mice significantly reduced food intake (˜92%), whereas this treatment did not alter food intake by wild-type mice (P=0.024) (FIG. 18B). These data suggest that the leanness of PRMT2^{-/-} mice, combined with lower concentrations of circulating leptin, constitute a form of leptin hypersensitivity.

Expression of PRMT2 in the Hypothalamus.

To determine localization of PRMT2 in the brain, in situ hybridizations were performed using ³⁵S-labeled riboprobes of mouse PRMT2 cDNA. PRMT2 mRNA was highly expressed in the ARC, ventromedial hypothalamic, paraventricular hypothalamic (PVH) and supraoptic (SON) nuclei (FIG. 19A, upper panel). PRMT2 mRNA was also abundant in paraventricular thalamic nucleus and pyramidal cell layer of the hippocampus. No signal was detected using the antisense probe and brains from PRMT2^{-/-} mice (FIG. 19A, lower panel), and the corresponding sense probe and wild-type brains (data not shown). Stat3 mRNA expression was equally observed in the arcuate (ARC), and paraventricular hypothalamic nuclei (PVH) of wild-type and PRMT2^{-/-} mice (FIG. 19B, upper and lower panel, respectively). Anorexigenic pro-opiomelanocortin (POMC) neurons have been shown to contain both Ob-R and Stat3 in the ARC (Hakansson et al. J. Neuroendocrinol. 18: 559-572 (1998); Hakansson et al. Neuroendocrinol. 68: 420-27 (1998)). PVN is also a prominent site of leptin receptor (Ob-R) and Stat3 expression, and these PVN can detect and integrate anorexigenic signals from the ARC (Hakansson et al. Neuroendocrinol. 68: 420-27 (1998); Cowley et al. Neuron 24: 155-63 (1999)). Therefore, the pattern of PRMT2 mRNA expression in the hypothalamus suggests that PRMT2 may affect appetite control and body weight through modulating central action of leptin mediated by Stat3.

STAT-3 Residue Arg-31 is a Substrate for Methylation by PRMT-2.

Stat3 plays a crucial role in the regulation of feeding and energy homeostasis by leptin (Bates et al. Trends Endocrinol. Metab. 14: 447-452 (2003)). It has recently been shown that Arg³¹ residue of Stat1 is a substrate for another protein arginine methyltransferase, PRMT1 (Mowen et al. Cell 104: 731-41 (2001). Several residues including the Arg³¹ residue are conserved in other members of the Stat family. Thus, Stat3 also contains an Arg³¹ residue.

Therefore tests were performed to ascertain whether PRMT2 might modulate leptin signaling through Stat3 methylation. To explore the possibility that Stat3 is a methylation substrate for PRMT2, an in vitro methylation assay was performed using a GST-Stat3 protein as substrate and Flag PRMT2 fusion proteins as enzyme sources. HEK293 cells were transiently transfected with either wild-type or mutated PRMT2 cDNA lacking functional Ado-Met domain (by substitution of alanine for the following amino acids, 145-GCGTG-149 SEQ ID NO:7). Flag fusion proteins were purified from cell lysates using an anti-Flag M2 affinity gel. The Flag fusion proteins were subjected to an in vitro methylation assay.

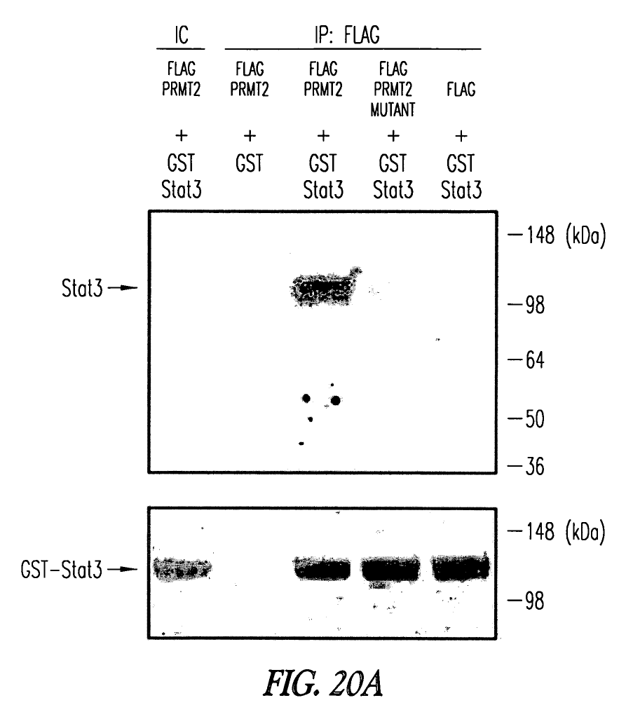

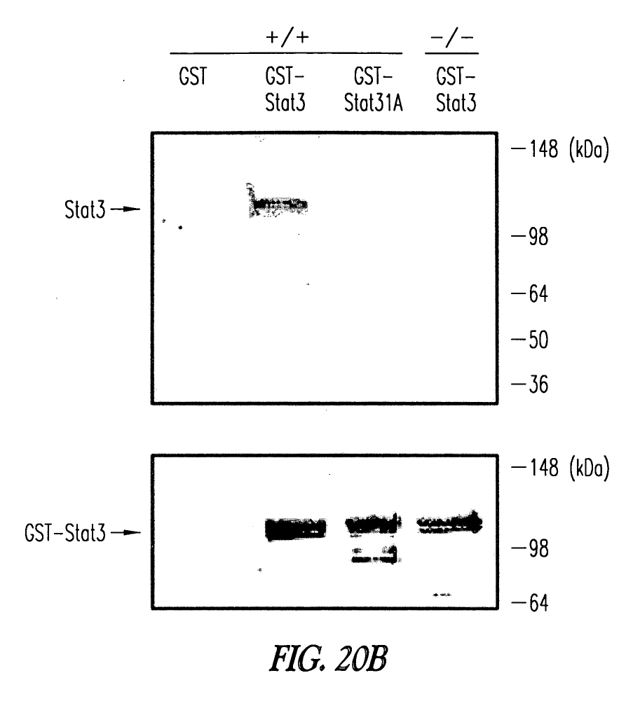

As shown in FIG. 20A (lane 3), Flag fusion protein from the cells transfected wild-type PRMT2-Flag cDNA could methylate GST-Stat3, whereas transfection of neither vector alone nor mutated PRMT2-Flag cDNA induced methylation of GST-Stat3 (FIG. 20A, lanes 4 and 5). To confirm the methyltransferase activity of PRMT2 in vitro, another in vitro methylation assay was performed using MEF extracts as a source of methyltransferase activity. Cell extracts from wild-type MEFs were also capable of methylating GST-Stat3, whereas cell extracts from PRMT2^{-/-} cells abolished Stat3 methylation (FIG. 20B, lanes 2 and 4). To determine whether PRMT2 might utilize the Stat3 Arg³¹ residue as a target for methylation, a GST-Stat3 mutant protein with Ala³¹ instead of Arg³¹ was also tested. This mutation resulted in substantially no methylation of Stat3 (FIG. 20B, lane 3), suggesting that Arg³¹ residue could be a target for Stat3 methylation by PRMT2.

Direct Association of PRMT-2 with STAT3 In Vivo.

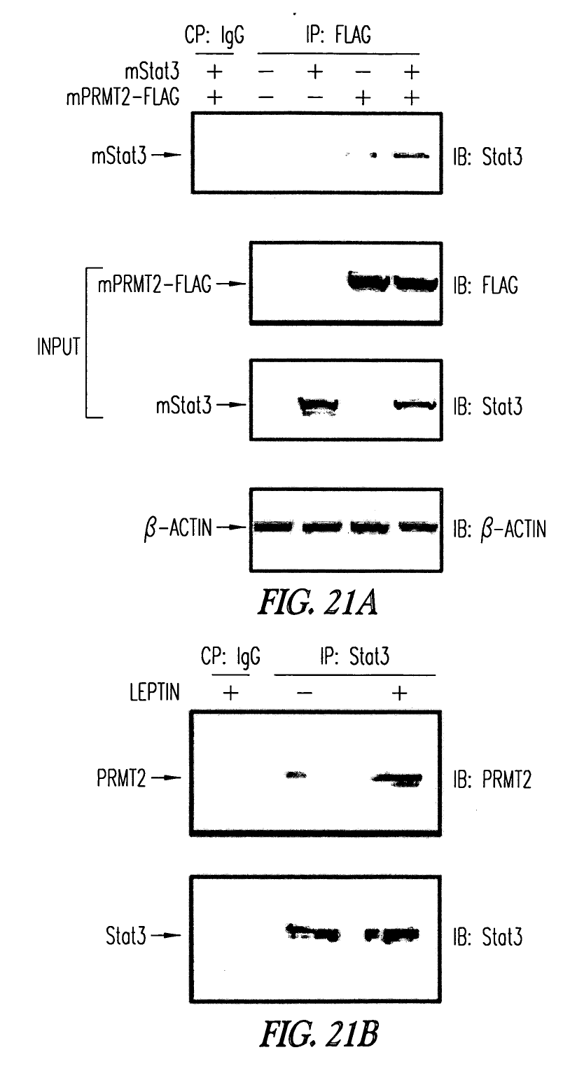

Previous in situ immunofluorescence data revealed that human PRMT-2 (HRMT1L1) was localized in both the nucleus and the cytoplasm (Kzhhyshkowska et al. Biochem. J. 358: 305-14 (2001). Identical results were observed by immunoblotting using mouse hypothalamic cells. Further experiments indicated that endogenous PRMT-2 was localized in the nucleus and cytoplasm of hypothalamic cells, whereas PRMT1 was predominantly localized in the nucleus (data not shown). These results indicate that PRMT-2 and STAT3 are colocalized in vivo and can form a complex in both Cytoplasmic and nuclear regions in vivo. To examine whether a direct interaction occurs between PRMT-2 and STAT3 in vivo, FLAG-tagged full-length PRMT-2 cDNA and/or STAT3 cDNA were transiently transfected into HEK293 cells. After culturing the transfected cells, an immunoprecipitation was performed on cell lysates using anti-Flag antibodies followed by immunoblotting with anti-PRMT-2 antibody. As shown in FIG. 21A, endogenous STAT3 was co precipitated in PRMT-2-transfected cell (FIG. 21A, lane 4), although the interaction between PRMT-2 and STAT3 was increased when STAT3 was cotransfected into the cells (FIG. 21A, lane 5). Identical results were observed when using anti-STAT3 antibodies for immunoprecipitation (data not shown).

A mouse hypothalamic cell line, GT1-7, was established that expressed both STAT3 and PRMT-2 (FIG. 21B), and used as a cell model for the study of leptin signaling. To further examine whether leptin stimulated endogenous interaction between PRMT-2 and STAT3, extracts from the cells that were untreated or treated with mouse leptin (100 nM) were subjected to immunoprecipitation with anti-STAT3 antibody, followed by immunoblotting with anti-PRMT-2 antibody. PRMT-2 was observed in the complexes from the untreated cells that were immunoprecipitated by anti-STAT3 antibodies (FIG. 21B, lane 2). However, treatment with leptin enhanced that amount PRMT-2 that co-precipitated with STAT3 (FIG. 21B, lane 3). PRMT-2 was not detectable in immunoprecipitates obtained with preimmune rabbit IgG (FIG. 21B, lane 1). These data indicate that endogenous PRMT-2 can directly interact with STAT3 in a ligand-dependent manner.

Modulation of STAT3 Methylation Through Ado-Met Domain of PRMT-2.

It has been previously been shown that PRMT-2 is not capable of methylating histone or many other proteins methylated by other protein arginine methyltransferases. However, PRMT-2 can bind S-adenosylmethionine through its AdoMet motif. To examine whether STAT3 is indeed arginine-methylated by PRMT-2 in vivo, transient transfections into HEK293 cells were initially performed using wild-type and mutated PRMT-2 cDNA lacking functional Ado-Met domain (the PRMT-2-4A mutant, in which residues ₁₄₁ILDV₁₄₄ were changed to four consecutive alanines to abolish methyltransferase activity. After incubation for 24 hr, immunoprecipitations were performed using anti-STAT3 antibody followed by immunoblotting with the α-methyl arginine antibody recognizing free and bound NG-NG-dimethyl or monomethyl arginine antibody.

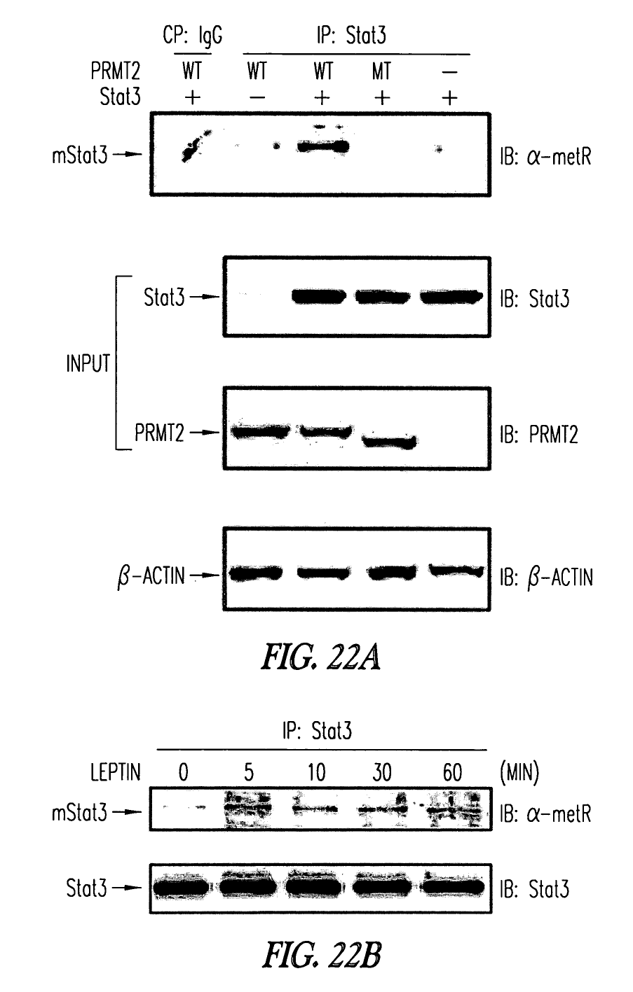

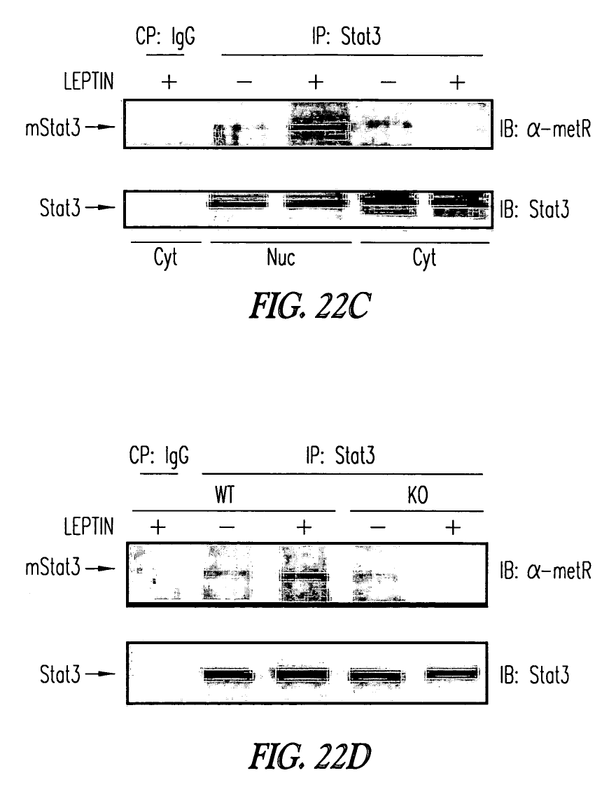

As shown in FIG. 22A, endogenous methylated STAT3 was detected in wild-type PRMT-2-transfected cell (lane 2), although methylation was increased when STAT3 was cotransfected into the cells (lane 3). However, transfection of mutant PRMT-2 and/or STAT3 alone failed to evoke STAT3 methylation (FIG. 22A, lanes 4 and 5). Wild-type PRMT-2 and the catalytically defective mutant PRMT-2 were expressed equally well (FIG. 22A, lane 2-4), but cotransfection of mutated PRMT-2 with STAT3 exhibited no appreciable effect on STAT3 methylation (FIG. 22A, lane 4). These data suggest that PRMT-2 requires the AdoMet motif to exhibit methyltransferase activity and methylate STAT3 in vivo.

To determine whether leptin induces endogenous STAT3 methylation in target tissues, extracts from mouse hypothalamic cells untreated or treated with mouse leptin (100 nM) were subjected to immunoprecipitation with STAT3 antibody followed by immunoblotting with antibodies directed against the α-methyl arginine. Methylation reactivity was detected in untreated GT1-7 cells (FIG. 22B, lane 1). After leptin stimulation, Stat3 methylation was remarkably increased at 5 min and sustained for 60 min (FIG. 22B, lane 2 to 5). Furthermore, to determine the localization of methylated Stat3 after leptin stimulation, both nuclear and cytoplasmic extracts from the cells were also subjected to the same immunoprecipitation experiment. Methylation of Stat3 was detected equally between nuclear and cytoplasmic extracts from the untreated cells (FIG. 22C, lane 2 and 4), although methylated Stat3 was remarkably increased and predominantly localized in the nucleus after leptin stimulation (FIG. 22C, lane 3 and 5). Since tyrosine-phosphorylated Stat3 stimulated by leptin also translocates to the nucleus, it is likely that, at least a part of the activated Stat3 is modified by both tyrosine phosphorylation and methylation in the nucleus.

Next, to determine whether the loss PRMT2 affects methylation of endogenous Stat3, leptin-induced Stat3 methylation was observed in tissue extracts and cells derived from both wild-type and PRMT2^{-/-} mice. Ob-Rb is predominantly expressed in hypothalamus, but is also detected in peripheral tissues. Leptin was reported to modulate vascular remodeling mediated by Ob-Rb in vascular smooth muscle cells (VSMC) (Parhami et al. Circ. Res. 88: 954-60 (2001); Schafer et al. Arterioscler. Thromb. Vasc. Biol. 24: 112-117 (2004). Protein expression PRMT2 as well as Ob-Rb was confirmed in VSMC, indicating that VSMC derived from PRMT2^{-/-} mice is a potential tool for determining the role of PRMT2 in Stat3 signaling. Indeed, leptin (100 nM) evoked a transient methylation of Stat3 at 10 min in wild-type VSMC (FIG. 22D, lane 2-5), whereas increased Stat3 methylation was not observed in PRMT2^{-/-} cells (FIG. 22D, lane 6-9). Taken together, these data suggests that endogenous PRMT2 may be essential for maximal methylation of Stat3.

Enhanced and Prolonged STAT3 Phosphorylation in PRMT2^{-/-} Tissues.

Previous studies indicate that arginine methylation of STAT1 is needed for tyrosine dephosphorylation of STAT1 in nuclei. Inhibition of STAT1 methylation resulted in a prolonged half-life for tyrosine-phosphorylated STAT1. Zhu et al. J. Biol. Chem. 277: 35787-90 (2002).

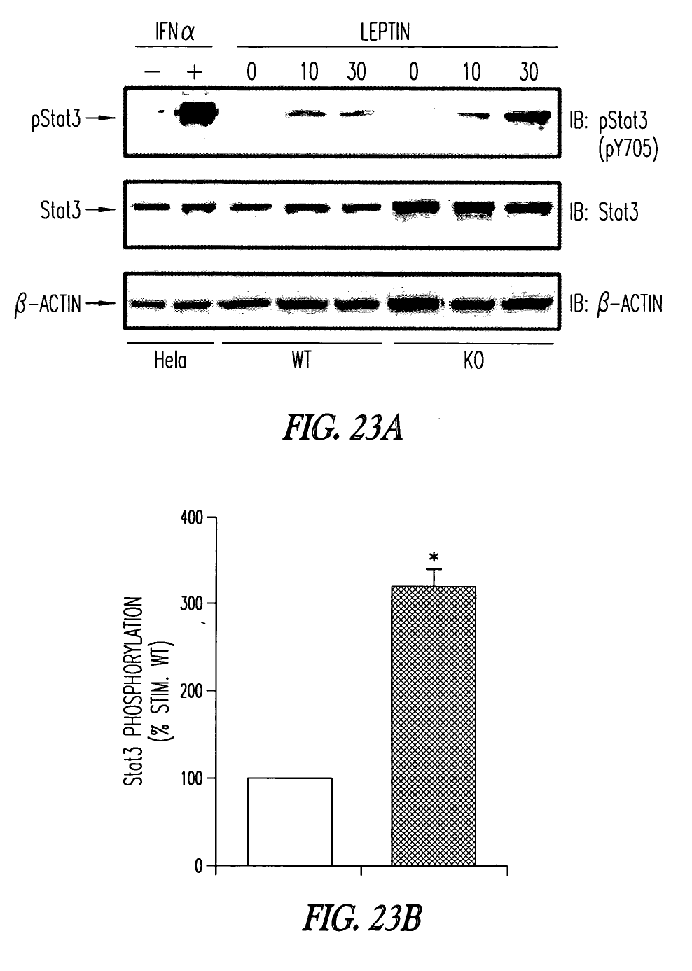

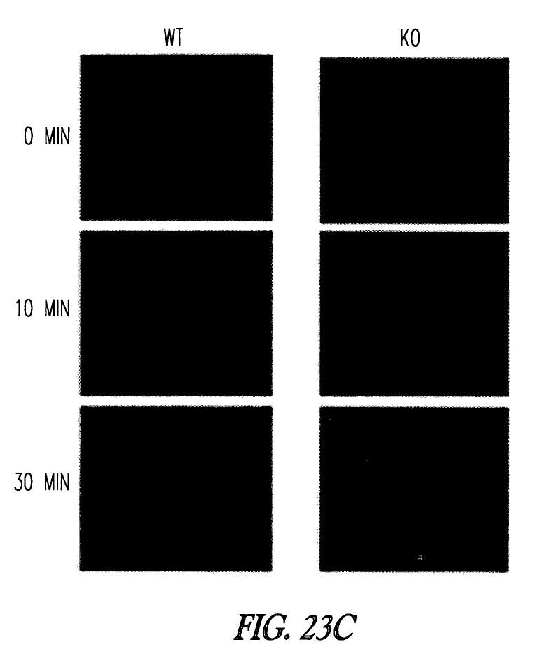

To determine whether the absence of PRMT-2 can modulate tyrosine phosphorylation of STAT3, tyrosine phosphorylated STAT3 was observed in the nuclei of wild-type and PRMT-2^{-/-} vascular smooth muscle cells. An apparent increased and sustained tyrosine phosphorylation of STAT3 was observed in nuclear extracts from PRMT-2^{-/-} cells at 30 min after stimulation with mouse leptin (FIGS. 23A and B). To further confirm that STAT3 tyrosine phosphorylation is sustained in PRMT^{-/-} cells, immunocytochemistry was performed using antibodies that recognize a phosphorylated Tyr⁷⁰⁵ residue of Stat3. At 10 min after stimulation with mouse leptin, no apparent difference of phosphorylated STAT3 localization was observed between wild-type and PRMT-2^{-/-} cells (FIG. 23C, middle images). However, 30 minutes after leptin stimulation, tyrosine phosphorylated STAT3 remained localized within the nucleus of PRMT-2^{-/-} cells in a more aggregated pattern than in wild type cells. In contrast, 30 minutes after leptin stimulation, tyrosine phosphorylation of Stat3 had declined in the nucleus of wild-type cells (FIG. 23C, lower lane). To focus on central leptin action, immunohistochemistry using brain sections was examined. In wild-type mice, tyrosine phosphorylation of hypothalamic Stat3 had peaked at 45 min after peripheral administration of leptin (1.67 μg/g body weight) and was gradually declining by 180 min after leptin treatment (data not shown). Immunoreactivity of tyrosine phosphorylated Stat3 was still observed in the ARC and VMH of wild-type mice at 90 min after leptin stimulation (FIG. 23D, left panel). However, the intensity of tyrosine phosphorylated Stat3 was more pronounced in the ARC and VMH of PRMT2^{-/-} mice at the same time point (FIG. 23D, right panel).

It has been reported that the single amino acid substitution of Arg³¹ for Ala in the Stat1 leads to a pivotal modulation of its activity in regulating the tyrosine dephosphorylation of Stat1 (Shuai et al. Mol. Cell. Biol. 16: 4932-41(1996)). To determine the possible involvement of tyrosine phosphatase in enhanced tyrosine phosphorylation of PRMT2^{-/-} cells, the effect of a phosphatase inhibitor, o-vanadate on leptin-stimulated tyrosine phosphorylation of Stat3 was examined. In nuclear extracts from the wild-type VSMC, pretreatment with o-vanadate further enhanced leptin-stimulated tyrosine phosphorylation of Stat3 (FIG. 23E, lane 1 and 2). However, no enhancement of tyrosine phosphorylation of Stat3 was observed in nuclear extracts from the vanadate-treated PRMT2^{-/-} VSMC—untreated PRMT2^{-/-} cells already exhibited increased tyrosine phosphorylation of Stat3 compared with that of the untreated wild-type cells (FIG. 23D lane 3 and 4). Taken together, these data suggest that PRMT2 knockout results in an enhanced and prolonged tyrosine phosphorylation of Stat3 after leptin stimulation in vivo and the enhancement in PRMT2^{-/-} cells and tissues is likely to be associated with tyrosine phosphatase activity.

Increased Hypothalamic POMC Expression in Leptin-Treated PRMT2^{-/-} Mice.

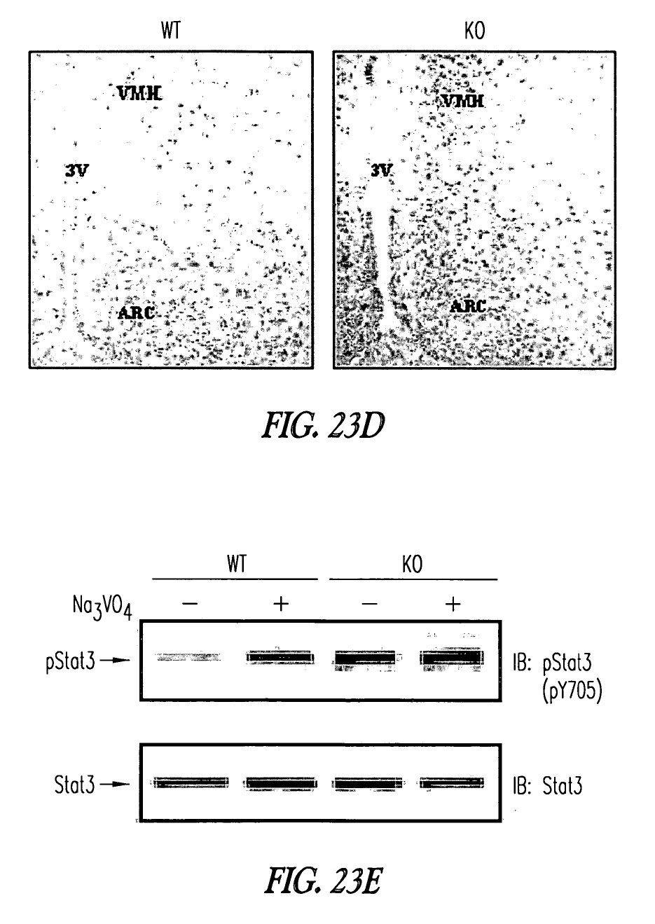

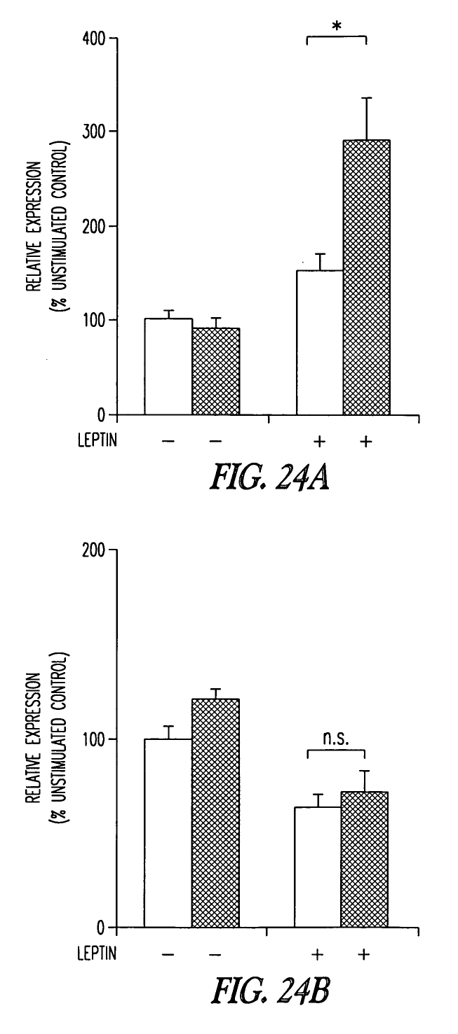

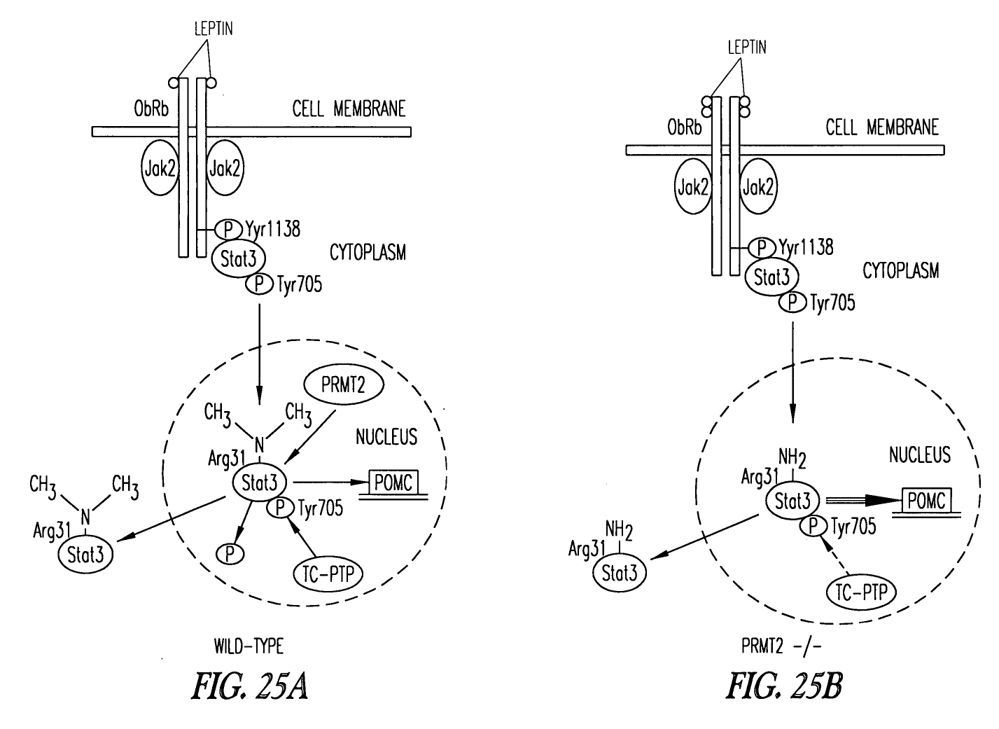

Upon leptin stimulation, Stat3 was rapidly tyrosine-phosphorylated and translocated to the nucleus, where phosphorylated Stat-3 promote the expression of target genes such as anorexigenic POMC and orexigenic NPY (Sahu Endocrinology 145: 2613-20 (2004). To determine whether the deletion of PRMT2 can affect the expression of hypothalamic POMC and NPY, mRNA expression was quantified by real-time PCR was performed. Compared with treatment with vehicle alone, administration of exogenous mouse leptin (0.5 μg/g body weight) was sufficient to induce weight loss in both wild-type (−0.12±0.16 g vs. −0.59±0.29 g; vehicle vs. leptin-treated wild-type mice) and PRMT2^{-/-} mice (−0.03±0.14 g vs. −1.0±0.2 g; vehicle vs. leptin-treated PRMT2^{-/-} mice). There was no statistical difference between the expression of hypothalamic POMC mRNA in untreated wild-type and PRMT2^{-/-} mice. However, while the expression of hypothalamic POMC mRNA in wild-type mice treated with leptin was increased by ˜53% compared with that of untreated mice (FIG. 24A), leptin-treated PRMT2^{-/-} mice showed a significantly higher level (˜90%) of hypothalamic POMC mRNA compared with leptin-treated wild-type mice (FIG. 24A). In contrast, to NPY mRNA expression in leptin-treated PRMT2^{-/-} mice, which was similar with that in wild-type control mice, hypothalamic NPY mRNA expression in PRMT2^{-/-} mice was significantly reduced by treatment with leptin (FIG. 24B).

It has been shown that an Ob-Rb-mediated Stat3 signal is required for the stimulation of POMC expression, whereas Stat3-independent signals triggered by Ob-Rb play a pivotal role on the regulation of NPY expression (Bates et al. Nature 421: 856-59 (2003)). Therefore, the present data suggest that loss of PRMT2 may sensitize Ob-Rb-Stat3-dependent signaling that mediates melanocortin function in the hypothalamus.

Therefore, as illustrated herein, PRMT2^{-/-} mice exhibited significant reductions of weight gain and food intake, and a marked decrease of glycogen storage in the liver. Leanness in PRMT2^{-/-} mice was accompanied by lower concentration of circulating leptin as well as lower concentrations of blood glucose, serum insulin and triglycerides. Resistance to food-dependent obesity in PRMT2^{-/-} mice was also indicated by lesser weight gain and lower accumulation of body fat on a high-fat feeding. After intraperitoneal administration of leptin, PRMT2^{-/-} mice lost weight and reduced food intake more than wild-type littermates did. In situ hybridization revealed that both PRMT2 and signal transducers and activators of transcription 3 (Stat3) mRNA were coexpressed in the hypothalamus including the arcuate, ventromedial hypothalamic and paraventricular hypothalamic nuclei. PRMT2 directly bound Stat3 and methylated arginine31 residue of Stat3 through its AdoMet domain in vivo and in vitro. Absence of PRMT2 resulted in a decrease methylation and a prolonged tyrosine phosphorylation of Stat3. mRNA expression of hypothalamic proopiomelanocortin was significantly increased in leptin-treated PRMT2^{-/-} mice in comparison with leptin treated wild-type controls. These results indicate that PRMT2 has a pivotal role in weight control through modulation of leptin-Stat3-melanocortin signaling. Thus, PRMT2 is a new target in the treatment of several metabolic disorders, such as food-dependent obesity, hyperlipidemia and type2 diabetes mellitus.

### REFERENCES

Chen, D. et al. Regulation of transcription by a protein methyltransferase. Science 284, 2174-2177 (1999).
Koh, S. S., Chen, D., Lee, Y. H. & Stallcup, M. R. Synergistic enhancement of nuclear receptor function by p160 coactivators and two coactivators with protein methyltransferase activities. J. Biol. Chem. 276, 1089-1098 (2001).
Mowen, K. A. et al. Arginine methylation of STAT1 modulates IFN□/□-induced transcription. Cell 104, 731-741 (2001).
4. Wang, H. et al. Methylation of histone H4 at arginine 3 facilitating transcriptional activation by nuclear hormone receptor. Science 293, 853-857 (2001).
5. Xu, W. et al. A transcriptional switch mediated by cofactor methylation. Science 294, 2507-2511 (2001).
6. Gary, J. D. & Clarke, S. RNA and protein interactions modulated by protein arginine methylation. Prog. Nucleic Acid Res. Mol. Biol. 61, 65-131 (1998).
7. Kagan, R. M. & Clarke, S. Widespread occurrence of three sequence motifs in diverse S-adenosylmethionine-dependent methyltransferases suggests a common structure for these enzymes. Arch Biochem. Biophys. 310, 417-427 (1994).
8. Lin, W. J., Gary, J. D., Yang, M. C., Clarke, S. & Herschman, H. R. The mammalian immediate-early TIS21 protein and the leukemia-associated BTG1 protein interact with a protein-arginine N-methyltransferase. J Biol. Chem. 271,15034-15044 (1996).
9. Abramovich, C., Yakobson, B., Chebath, J. & Revel, M. A protein-arginine methyltransferase binds to the intracytoplasmic domain of the IFNAR1 chain in the type I interferon receptor. EMBO J 16, 260-266 (1997).
10. Katsanis, N., Yaspo, M. L. & Fisher, E. M. Identification and mapping of a novel human gene, HRMT1L1, homologous to the rat protein arginine N-methyltransferase 1 (PRMT1) gene. Mammalian Genome 8, 526-529 (1997).
11. Tang, J., Gary, J. D., Clarke, S. & Herschman, H. R. PRMT3, a type-1 protein arginine N-methyltransferase that differs from PRMT1 in its oligomerization, subcellular localization, substrate specificity, and regulation. J Biol. Chem. 273, 16935-16945 (1998).
12. Scott, H. S. et al. Identification and characterization of two putative human arginine methyltransferases (HRMT1L1 and HRMT1L2). Genomics 48, 330-340 (1998).
13. Pollack, B. P. et al. The human homologue of the yeast proteins Skb1 and Hs17p interacts with Jak kinases and contains protein methyltransferase activity. J Biol. Chem. 274, 31531-31542 (1999).
14. Aletta, J. M. Protein methylation: a signal event in post-translational modification. TIBS 23, 89-91 (1998).
15. Bauer, U. M., Daujat, S., Nielsen, S. J., Nightingale, K. & Kouzarides, T. Methylation at arginine 17 of histone H3 is linked to gene activation. EMBO Reports 3, 39-44 (2002).
16. Nabel, G. & Baltimore, D. An inducible transcription factor activates expression of human immunodeficiency virus in T cells. Nature 326, 711-713 (1987).
17. Sen, R. & Baltimore, D. Inducibility of kappa immunoglobulin enhancer-binding protein NF-κB by a posttranslational mechanism. Cell 47, 921-928 (1986).
18. Arenzana-Seisdedos, F. et al. Nuclear localization of I□B-□ promotes active transport of NF-κB from the nucleus to the cytoplasm. J. Cell Sci. 110, 369-378 (1997).
19. Arenzana-Seisdedos, F. et al. Inducible nuclear expression of newly synthesized IkB-□ negatively regulates DNA-binding and transcriptional activity of NF-kB. Mol. Cell. Biol. 15, 2689-2696 (1995).
20. Rodriguez, M. S., Thompson, J., Hay, R. T. & Dargemont, C. Nuclear retention of I□B□ protects it from signal induced degradation and inhibits nuclear factor □B transcriptional activation. J Biol. Chem. 274, 9108-9115 (1999).
21. Tam, W. F., Lee, L. H., Davis, L. & Sen, R. Cytoplasmic sequestration of Rel proteins by I□B□ requires CRM1-dependent nuclear export. Mol. Cell. Biol. 20, 2269-2284 (2000).
22. Wang, C.-Y., Mayo, M. W. & Baldwin, A. S. TNF- and cancer therapy-induced apoptosis: potentiation by inhibition of NF-kB. Science 274, 784-787 (1996).
23. Beg, A. A. & Baltimore, D. An essential role for NF-□B in preventing TNF-□-induced cell death. Science 274, 782-789 (1996).
24. van Antwerp, D. J., Martin, S. J., Kafri, T., Green, D. R. & Verma, I. M. Suppression of TNF-α-induced apoptosis of NF-κB. Science 274, 787-789 (1996).
25. Wang, C.-Y., Cusack, J. C., Liu, R. & Baldwin, A. S. Control of inducible chemoresistance: Enhanced anti-tumor therapy through increased apoptosis by inhibition of NF-□B. Nat. Med. 5, 412-417 (1999).
26. Brown, K., Park, S., Kanno, T., Franzoso, G. & Siebenlist, U. Mutual regulation of the transcription activator NF-kB and its inhibitor IkB-□. Proc. Natl. Acad. Sci. USA 90, 2532-2536 (1993).
27. Sun, S. C., Ganchi, P. A., Ballard, D. W. & Greene, W. C. NF-kappa B controls expression of inhibitor I kappa B alpha: evidence for an inducible autoregulatory pathway. Science 259, 1912-1915 (1993).
28. Chiao, P. J., Miyamoto, S. & Verma, I. M. Autoregulation of IκB-□ activity. Proc. Natl. Acad. Sci. USA 91, 28-32 (1994).
29. Liu, Z.-G., Hsu, H., Goeddel, D. V. & Karin, M. Dissection of TNF receptor 1 effector functions: JNK activation is not linked to apoptosis while NF-kB activation prevents cell death. Cell 87, 565-576 (1996).
30. Karin, M. & Lin, A. NF-κB at the crossroads of life and death. Nat. Immunol. 3, 221-227 (2002).
31. Leung, K. & Nabel, G. J. HTLV-I transactivator induces interleukin-2 receptor expression through an NF-□B-like factor. Nature 333, 776-778 (1988).
32. Markovitz, D. M. et al. Differential regulation of human immunodeficiency viruses: a novel HIV-2 regulatory element responds to stimulation of the T-cell antigen receptor. Proc. Natl. Acad. Sci. USA 87, 9098-9102 (1990).
33. Duckett, C. S. et al. Dimerization of NF-KB2 with RelA(p65) regulates DNA binding, transcriptional activation, and inhibition by an I□B-□ (MAD-3). Mol. Cell. Biol. 13, 1315-1322 (1993).
34. Danthinne, X., Aoki, K., Kurachi, A. L., Nabel, G. J. & Nabel, E. G. Combination gene delivery of the cell-cycle inhibitor p27 with thymidine kinase enhances prodrug cytotoxicity. J. Virol. 72, 9201-9207 (1998).
35. Wu, B., Woffendin, C., MacLachlan, I. & Nabel, G. J. Distinct domains of IkB-□ inhibit human immunodeficiency virus type 1 replication through NF-kB and Rev. J. Virol. 71, 3161-3167 (1997).
36. Dignam, J. D., Lebovitz, R. M. & Roeder, R. G. Accurate transcription initiation by RNA polymerase II in a soluble extract from isolated mammalian nuclei. Nucleic Acids Res. 11, 1475-1489 (1983).
37. Perkins, N. D. et al. Regulation of NF-□B by cyclin-dependent kinases associated with the p300 co-activator. Science 275, 523-527 (1997).
38. Smith, D. B. & Johnson, K. S. Single-step purification of polypeptides expressed in Escherichia coli as fusions with glutathione S-transferase. Gene 67, 31-40 (1988).
39. Abramovich, C., Yakobson, B., Chebath, J. and Revel, M. (1997) A protein-arginine methyltransferase binds to the intracytoplasmic domain of the IFNAR1 chain in the type I interferon receptor. Embo J, 16, 260-6.
Aleta, J. M., Cimato, T. R. and Ettinger, M. J. (1998) Protein methylation: a signal event in post-translational modification. Trends in Biochemical Sciences, 23, 89-91.
Bauer, U. M., Daujat, S., Nielsen, S. J., Nightingale, K. and Kouzarides, T. (2002) Methylation at arginine 17 of histone H3 is linked to gene activation. Embo Reports, 3, 39-44.
Chen, D., Ma, H., Hong, H., Koh, S. S., Huang, S. M., Schurter, B. T., Aswad, D. W. and Stallcup, M. R. (1999) Regulation of transcription by a protein methyltransferase. Science, 284, 2174-7.
Cole, S. E., Wiltshire, T. and Reeves, R. H. (1998) Physical mapping of the evolutionary boundary between human chromosomes 21 and 22 on mouse chromosome 10. Genomics, 50, 109-11.
Dahiya, A., Wong, S., Gonzalo, S., Gavin, M. and Dean, D. C. (2001) Linking the Rb and polycomb pathways. Molecular Cell, 8, 557-568.
Danthinne, X., Aoki, K., Kurachi, A. L., Nabel, G. J. and Nabel, E. G. (1998) Combination gene delivery of the cell cycle inhibitor p27 with thymidine kinase enhances prodrug cytotoxicity. J Virol, 72, 9201-7.
Dick, F. A., Sailhamer, E. and Dyson, N. J. (2000) Mutagenesis of the pRB pocket reveals that cell cycle arrest functions are separable from binding to viral oncoproteins. Molecular and Cellular Biology, 20, 3715-3727.
Dyson, N. (1998) The regulation of E2F by pRB-family proteins. Genes Dev, 12, 2245-62.
Emami, K. H. and Carey, M. (1992) A Synergistic Increase in Potency of a Multimerized Vp16 Transcriptional Activation Domain. Embo Journal, 11, 5005-5012.
Fabbrizio, E., El Messaoudi, S., Polanowska, J., Paul, C., Cook, J. R., Lee, J. H., Negre, V., Rousset, M., Pestka, S., Le Cam, A. and Sardet, C. (2002) Negative regulation of transcription by the type II arginine methyltransferase PRMT5. Embo Reports, 3, 641-645.
Harbour, J. W. and Dean, D. C. (2000) The Rb/E2F pathway: expanding roles and emerging paradigms. Genes & Development, 14, 2393-2409.
Herrera, R. E., Sah, V. P., Williams, B. O., Makela, T. P., Weinberg, R. A. and Tyler, J. (1996) Altered cell cycle kinetics, gene expression, and G(1), restriction point regulation in Rb-deficient fibroblasts. Molecular and Cellular Biology, 16, 2402-2407.
Kagan, R. M. and Clarke, S. (1994) Widespread occurrence of three sequence motifs in diverse S-adenosylmethionine-dependent methyltransferases suggests a common structure for these enzymes [published erratum appears in Arch Biochem Biophys 1995 Jan. 10; 316(1):657]. Arch Biochem Biophys, 310, 417-27.
Katsanis, N., Yaspo, M. L. and Fisher, E. M. (1997) Identification and mapping of a novel human gene, HRMT1L1, homologous to the rat protein arginine N-methyltransferase 1 (PRMT1) gene. Mamm Genome, 8, 526-9.
Koh, S. S., Chen, D. G., Lee, Y. H. and Stallcup, M. R. (2001) Synergistic enhancement of nuclear receptor function by p160 coactivators and two coactivators with protein methyltransferase activities. Journal of Biological Chemistry, 276, 1089-1098.
Le Cam, L., Polanowska, J., Fabbrizio, E., Olivier, M., Philips, A., Eaton, E. N., Classon, M., Geng, Y. and Sardet, C. (1999) Timing of cyclin E gene expression depends on the regulated association of a bipartite repressor element with a novel E2F complex. Embo Journal, 18, 1878-1890.
Lin, W. J., Gary, J. D., Yang, M. C., Clarke, S. and Herschman, H. R. (1996) The mammalian immediate-early TIS21 protein and the leukemia-associated BTG1 protein interact with a protein-arginine N-methyltransferase. J Biol Chem, 271, 15034-44.
Luo, R. X., Postigo, A. A. and Dean, D. C. (1998) Rb interacts with histone deacetylase to repress transcription. Cell, 92, 463-473.
McBride, A. E. and Silver, P. A. (2001) State of the arg. protein methylation at arginine comes of age. Cell, 106, 5-8.
Mowen, K. A., Tang, J., Zhu, W., Schurter, B. T., Shuai, K., Herschman, H. R. and David, M. (2001) Arginine methylation of STAT1 modulates IFN alpha/beta-induced transcription. Cell, 104, 731-741.
Nielsen, S. J., Schneider, R., Bauer, U. M., Bannister, A. J., Morrison, A., O'Carroll, D., Firestein, R., Cleary, M., Jenuwein, T., Herrera, R. E. and Kouzarides, T. (2001) Rb targets histone H3 methylation and HP1 to promoters. Nature, 412, 561-565.
Qi, C., Chang, J., Zhu, Y. W., Yeldandi, A. V., Rao, S. M. and Zhu, Y. J. (2002) Identification of protein arginine methyltransferase 2 as a coactivator for estrogen receptor alpha. Journal of Biological Chemistry, 277, 28624-28630.
Nielsen, S. J., Schneider, R., Bauer, U. M., Bannister, A. J., Morrison, A., O'Carroll, D., Firestein, R., Cleary, M., Jenuwein, T., Herrera, R. E. and Kouzarides, T. (2001) Rb targets histone H3 methylation and HP1 to promoters. Nature, 412, 561-565.
Robertson, K. D., Ait-Si-Ali, S., Yokochi, T., Wade, P. A., Jones, P. L. and Wolffe, A. P. (2000) DNMT1 forms a complex with Rb, E2F1 and HDAC1 and represses transcription from E2F-responsive promoters. Nature Genetics, 25, 338-342.
Scott, H. S., Antonarakis, S. E., Lalioti, M. D., Rossier, C., Silver, P. A. and Henry, M. F. (1998) Identification and characterization of two putative human arginine methyltransferases (HRMT1L1 and HRMT1L2). Genomics, 48, 330-40.
Tang, J., Frankel, A., Cook, R. J., Kim, S., Paik, W. K., Williams, K. R., Clarke, S. and Herschman, H. R. (2000) PRMT1 is the predominant type I protein arginine methyltransferase in mammalian cells. J Biol Chem, 275, 7723-30.
Tang, J., Gary, J. D., Clarke, S. and Herschman, H. R. (1998) PRMT 3, a type I protein arginine N-methyltransferase that differs from PRMT1 in its oligomerization, subcellular localization, substrate specificity, and regulation. J Biol Chem, 273, 16935-45.
Tanner, F. C., Yang, Z. Y., Duckers, E., Gordon, D., Nabel, G. J. and Nabel, E. G. (1998) Expression of cyclin-dependent kinase inhibitors in vascular disease. Circ Res, 82, 396-403.
Trimarchi, J. M. and Lees, J. A. (2002) Sibling rivalry in the E2F family. Nature Reviews Molecular Cell Biology, 3, 11-20.
Vandel, L., Nicolas, E., Vaute, O., Ferreira, R., Ait-Si-Ali, S. and Trouche, D. (2001) Transcriptional repression by the retinoblastoma protein through the recruitment of a histone methyltransferase. Molecular and Cellular Biology, 21, 6484-6494.
Wang, H. B., Huang, Z. Q., Xia, L., Feng, Q., Erdjument-Bromage, H., Strahl, B. D., Briggs, S. D., Allis, C. D., Wong, J. M., Tempst, P. and Zhang, Y. (2001) Methylation of histone H4 at arginine 3 facilitating transcriptional activation by nuclear hormone receptor. Science, 293, 853-857.
Weiss, V. H., McBride, A. E., Soriano, M. A., Filman, D. J., Silver, P. A. and Hogle, J. M. (2000) The structure and oligomerization of the yeast arginine methyltransferase, Hmt1. Nature Structural Biology, 7, 1165-1171.
Xu, W., Chen, H. W., Du, K. Y., Asahara, H., Tini, M., Emerson, B. M., Montminy, M. and Evans, R. M. (2001) A transcriptional switch mediated by cofactor methylation. Science, 294, 2507-2511.
Zhang, H. S., Gavin, M., Dahiya, A., Postigo, A. A., Ma, D., Luo, R. X., Harbour, J. W. and Dean, D. C. (2000a) Exit from G1 and S phase of the cell cycle is regulated by repressor complexes containing HDAC-Rb-hSWI/SNF and Rb-hSWI/SNF. Cell, 101, 79-89.
Zhang, X., Zhou, L. and Cheng, X. (2000b) Crystal structure of the conserved core of protein arginine methyltransferase PRMT3. Embo J, 19, 3509-19.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A method for modulating NFκB, E2F1 or STAT3 activity in a mammalian cell that comprises administering to the mammalian cell a Protein Arginine N-Methyltransferase-2 polypeptide comprising SEQ ID NO:2, 3 or 6 or a Protein Arginine N-Methyltransferase-2 nucleic acid that encodes the Protein Arginine N-Methyltransferase-2 polypeptide.

2. The method of claim 1, wherein the Protein Arginine N-Methyltransferase-2 nucleic acid comprises SEQ ID NO:1.

3. The method of claim 1, wherein the Protein Arginine N-Methyltransferase-2 polypeptide consists of SEQ ID NO:3 or 6

4. The method of claim 1, wherein the mammalian cell is in a mammal.

5. The method of claim 4, wherein NFκB or E2F1 activity is modulated to treat a disease or condition in the mammal.

6. The method of claim 5, wherein the disease or condition is an inflammation, allergy, cancer, HIV infection, adult respiratory distress syndrome, asthma, allograft rejection, vasculitis, or vascular restenosis.

7. The method of claim 5, wherein the disease or condition is a cancer or tumor.

8. The method of claim 7, wherein the cancer or tumor is a bladder carcinoma, breast carcinoma, colon carcinoma, kidney carcinoma, liver carcinoma, lung carcinoma, small cell lung cancer, esophagus carcinoma, gall-bladder carcinoma, ovary carcinoma, pancreas carcinoma, stomach carcinoma, cervix carcinoma, thyroid carcinoma, prostate carcinoma, skin carcinoma, squamous cell carcinoma, hematopoietic tumor, leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma, Burkett's lymphoma, hematopoietic tumor, acute myelogenous leukemia, chronic myelogenous leukemia, myelodysplastic syndrome, promyelocytic leukemia, mesenchymal tumor, fibrosarcoma, Rhabdomyosarcoma, central nervous system tumor, peripheral nervous system tumor, astrocytoma, neuroblastoma, glioma, schwannoma, melanoma, seminoma, teratocarcinoma, osteosarcoma, xeroderma pigmentosum, keratoxanthoma, thyroid follicular cancer or Kaposi's sarcoma.

9. The method of claim 4, wherein NFκB activity is modulated to promote apoptosis of the cell in the mammal.

10. The method of claim 4, which further comprises administering to the mammal a cytokine or cytotoxin.

11. The method of claim 1, wherein NFκB or E2F1 activity consists of inhibiting Protein Arginine N-Methyltransferase-2 activity or expression in the mammalian cell.

12. The method of claim 11, that comprises administering to the mammalian cell an antibody or nucleic acid that can inhibit the activity or expression of Protein Arginine N-Methyltransferase-2

13. The method of claim 12, wherein the nucleic acid that can inhibit the activity or expression is an siRNA, antisense nucleic acid or ribozyme that is selectively hybridizable under physiological conditions to an RNA derived from a DNA comprising SEQ ID NO:1.

14. The method of claim 11, wherein the mammalian cell is in a mammal.

15. The method of claim 14, wherein Protein Arginine N-Methyltransferase-2 expression is modulated to treat a disease.

16. The method of claim 15, wherein the disease is obesity, diabetes, hyperlipidemia, or insulin insensitivity.

17. A method of promoting weight loss in a mammal comprising administering to the mammal an agent that inhibits Protein Arginine N-Methyltransferase-2 expression or activity.

18. The method of claim 17, wherein the agent is an antibody or nucleic acid that can inhibit the activity or expression of Protein Arginine N-Methyltransferase-2

19. The method of claim 18, wherein the nucleic acid that can inhibit the activity or expression is an siRNA, antisense nucleic acid or ribozyme that is selectively hybridizable under physiological conditions to an RNA derived from a DNA comprising SEQ ID NO:1.

20. A method for inhibiting transcription from an HIV-1 LTR in a mammal that comprises administering to the mammal a therapeutically effective amount of a Protein Arginine N-Methyltransferase-2 polypeptide comprising SEQ ID NO:2, 3 or 6.

21. A method for inhibiting transcription from an HIV-1 LTR in a mammalian cell that comprises contacting the mammalian cell with amount of a Protein Arginine N-Methyltransferase-2 polypeptide comprising SEQ ID NO:3 or 6.

22. A method for identifying a test agent that can modulate Protein Arginine N-Methyltransferase-2 expression in a test cell comprising:
(a) contacting the test cell with a test agent; and
(b) observing whether expression of a nucleic acid comprising SEQ ID NO:1 is modulated relative to expression of a nucleic acid comprising SEQ ID NO:1 in a control cell that was not contacted with the test agent;
(c) observing whether Protein Arginine N-Methyltransferase-2 activity is modulated relative to Protein Arginine N-Methyltransferase-2 activity in a control cell that was not contacted with the test agent;
(d) observing whether NFκB activity is modulated relative to NFκB activity in a control cell that was not contacted with the test agent; or
(e) observing whether E2F activity is modulated relative to E2F activity in a control cell that was not contacted with the test agent.

23. The method of claim 22, wherein the test cell is a cancer cell or an immune cell.

24. The method of claim 22, wherein the test cell is a cultured cell that has been exposed to an interleukin or a cytokine to induce an inflammatory response.

25. An isolated Protein Arginine N-Methyltransferase-2 polypeptide comprising amino acid sequence SEQ ID NO:3, 4 or 6.

26. An isolated nucleic acid encoding the polypeptide of claim 25, wherein the polypeptide consists of amino acid sequence SEQ ID NO:3, 4 or 6.

27. An expression vector that comprises the nucleic acid of claim 26.

28. An isolated cell comprising the nucleic acid of claim 26.


SEQUENCE LISTING (47 sequences)

SEQ ID NO: 1 - DNA, 2093 bp, Homo sapiens
cactgcgcttgcgcgggttgagggcggtggctcagtctcctggaaaggaccgtccacccctccgcgctggcggtgtggacgcggaactcagcggagaaacgcgattgagaaatggaaaagaaaatgaaataaatcagcagttatgaggcagagcctaagagaactatggcaacatcaggtgactgtcccagaagtgaatcgcagggagaagagcctgctgagtgcagtgaggcgggtctcctgcaggagggagtacagccagaggagtttgtggccatcgcggactacgctgccaccgatgagacccagctcagttttttgagaggagaaaaaattcttatcctgagacaaaccactgcagattggtggtggggtgagcgtgcgggctgctgtgggtacattccggcaaaccatgtggggaagcacgtggatgagtacgaccccgaggacacgtggcaggatgaagagtacttcggcagctatggaactctgaaactccacttggagatgttggcagaccagccacgaacaactaaataccacagtgtcatcctgcagaataaagaatccctgacggataaagtcatcctggacgtgggctgtgggactgggatcatcagtctcttctgtgcacactatgcgcggcctagagcggtgtacgcggtggaggccagtgagatggcacagcacacggggcagctggtcctgcagaacggctttgctgacatcatcaccgtgtaccagcagaaggtggaggatgtggtgctgcccgagaaggtggacgtgctggtgtctgagtggatggggacctgcctgctgtttgagttcatgatcgagtccatcctgtatgcccgggatgcctggctgaaggaggacggggtcatttggcccaccatggctgcgttgcaccttgtgccctgcagtgctgataggattatcgtagccaaggtgctcttctgggacaacgcgtacgagttcaacctcagcgctctgaaatctttagcagttaaggagtttttttcaaagcccaagtataaccacattttgaaaccagaagactgtctctctgaaccgtgcactatattgcagttggacatgagaaccgtgcaaatttctgatctagagaccctgaggggcgagctgcgcttcgacatcaggaaggcggggaccctgcacggcttcacggcctggtttagcgtccacttccagagcctgcaggaggggcagccgccgcaggtgctcagcacggggcccttccaccccaccacacactggaagcagacgctgttcatgatggacgacccagtccctgtccatacaggagacgtggtcacgggttcagttgtgttgcagagaaacccagtgtggagaaggcacatgtctgtggctctgagctgggctgtcacttccagacaagaccccacatctcaaaaagttggagaaaaagtcttccccatctggagatgacagttgatgctttatttggaaagcagtgtgcatatcttgaggggtgatgaacacaagcaaaccaagttgcacctggcttctgcacactcctgcgaaagtcggtgaacattcactccacattgacccctccctagcctggcaggtgacgtcagggtccttcacagacaaacacgcttgggctcggcaggagctgccgtggccacccccgctgcccagtgtctgccctctagaagtaggctgtgtttccaggtgttcacccgtggtgcccacagtgccgacccgtggctgggtcggagctccatgttcctaagctaggtctaggtctacactcctaggacgcacgcatatcagcccgtgtaccctgtgacagtgactgtccccacctcctgtgttagtggtgcccttactgccgtcgctcatccactcgtgtgggacgtaggattgcacagggctgtgccagtggcgtgtagggaacactgccctggctcagcgtgcgagctaaggtggcgatgtatgcgatgggactctgcatgggatagtacagttgtgtagacgtcttccaaataaattatgtgttggtgccatcgcacatgctcaataaatattttaaatgagtgaaaaaaaaa

SEQ ID NO: 2 - protein, 433 aa, Homo sapiens
MATSGDCPRSESQGEEPAECSEAGLLQEGVQPEEFVAIADYAATDETQLSFLRGEKILILRQTTADWWWGERAGCCGYIPANHVGKHVDEYDPEDTWQDEEYFGSYGTLKLHLEMLADQPRTTKYHSVILQNKESLTDKVILDVGCGTGIISLFCAHYARPRAVYAVEASEMAQHTGQLVLQNGFADIITVYQQKVEDVVLPEKVDVLVSEWMGTCLLFEFMIESILYARDAWLKEDGVIWPTMAALHLVPCSADKDYRSKVLFWDNAYEFNLSALKSLAVKEFFSKPKYNHILKPEDCLSEPCTILQLDMRTVQISDLETLRGELRFDIRKAGTLHGFTAWFSVHFQSLQEGQPPQVLSTGPFHPTTHWKQTLFMMDDPVPVHTGDVVTGSVVLQRNPVWRRHMSVALSWAVTSRQDPTSQKVGEKVFPIWR

SEQ ID NO: 3 - protein, 218 aa, Artificial Sequence
  A synthetic PRMT-2-A mutant polypeptide
MATSGDCPRSESQGEEPAECSEAGLLQEGVQPEEFVAIADYAATDETQLSFLRGEKILILRQTTADWWWGERAGCCGYIPANHVGKHVDEYDPEDTWQDEEYFGSYGTLKLHLEMLADQPRTTKYHSVILQNKESLTDKVILDVGCGTGIISLFCAHYARPRAVYAVEASEMAQHTGQLVLQNGFADIITVYQQKVEDVVLPEKVDVLVSEWMGTCLL

SEQ ID NO: 4 - protein, 95 aa, Artificial Sequence
  A synthetic PRMT-2-N polypeptide
MATSGDCPRSESQGEEPAECSEAGLLQEGVQPEEFVAIADYAATDETQLSFLRGEKILILRQTTADWWWGERAGCCGYIPANHVGKHVDEYDPED

SEQ ID NO: 5 - protein, 4 aa, Homo sapiens
ILDV

SEQ ID NO: 6 - protein, 433 aa, Artificial Sequence
  A synthetic PRMT-2-4A mutant polypeptide
MATSGDCPRSESQGEEPAECSEAGLLQEGVQPEEFVAIADYAATDETQLSFLRGEKILILRQTTADWWWGERAGCCGYIPANHVGKHVDEYDPEDTWQDEEYFGSYGTLKLHLEMLADQPRTTKYHSVILQNKESLTDKVAAAAGCGTGIISLFCAHYARPRAVYAVEASEMAQHTGQLVLQNGFADIITVYQQKVEDVVLPEKVDVLVSEWMGTCLLFEFMIESILYARDAWLKEDGVIWPTMAALHLVPCSADKDYRSKVLFWDNAYEFNLSALKSLAVKEFFSKPKYNHILKPEDCLSEPCTILQLDMRTVQISDLETLRGELRFDIRKAGTLHGFTAWFSVHFQSLQEGQPPQVLSTGPFHPTTHWKQTLFMMDDPVPVHTGDVVTGSVVLQRNPVWRRHMSVALSWAVTSRQDPTSQKVGEKVFPIWR

SEQ ID NO: 7 - protein, 5 aa, Artificial Sequence
  synthetic peptide
GCGTG

SEQ ID NO: 8 - DNA, 32 bp, Artificial Sequence
  A synthetic primer
aagtcgacgccatggcaacatcaggtgactgt

SEQ ID NO: 9 - DNA, 33 bp, Artificial Sequence
  A synthetic primer
aagcggccgcttatctccagatggggaagactt

SEQ ID NO: 10 - DNA, 28 bp, Artificial Sequence
  A synthetic primer
aaggatccgcgaactgcatcatggagaa

SEQ ID NO: 11 - DNA, 28 bp, Artificial Sequence
  A synthetic primer
aaaagcttaaaccgcctaggaacgctca

SEQ ID NO: 12 - DNA, 71 bp, Artificial Sequence
  A synthetic primer
aagatatcgccatggacgagccagaactgtcggacagcggggacgaggccgcctgggaggatgaggacgat

SEQ ID NO: 13 - DNA, 34 bp, Artificial Sequence
  A synthetic primer
aatctagattactggagaccataagtttgagttg

SEQ ID NO: 14 - DNA, 32 bp, Artificial Sequence; identical to SEQ ID NO: 8
  A synthetic primer

SEQ ID NO: 15 - DNA, 33 bp, Artificial Sequence
  A synthetic primer
aatctagattaaaatgaatcacgcacgaccctt

SEQ ID NO: 16 - DNA, 59 bp, Artificial Sequence
  A synthetic primer
ataaagaatccctgacggataaagccgcagccgcggtgggctgtgggactgggatcatc

SEQ ID NO: 17 - DNA, 36 bp, Artificial Sequence
  A synthetic primer
gcgcgcgatatcgccatggcaacatcaggtgactgt

SEQ ID NO: 18 - DNA, 62 bp, Artificial Sequence
  A synthetic primer
gcgcgctctagactaggcatagtcaggcacgtcataaggataggggtcgtactcatccacgt

SEQ ID NO: 19 - DNA, 28 bp, Artificial Sequence
  A synthetic primer
aaggatccgcgaactgcatcatggagaa

SEQ ID NO: 20 - DNA, 28 bp, Artificial Sequence
  A synthetic primer
aaaagcttaaaccgcctaggaacgctca

SEQ ID NO: 21 - DNA, 32 bp, Artificial Sequence; identical to SEQ ID NO: 8
  A synthetic primer

SEQ ID NO: 22 - DNA, 33 bp, Artificial Sequence; identical to SEQ ID NO: 9
  A synthetic primer

SEQ ID NO: 23 - DNA, 71 bp, Artificial Sequence; identical to SEQ ID NO: 12
  A synthetic primer

SEQ ID NO: 24 - DNA, 34 bp, Artificial Sequence; identical to SEQ ID NO: 13
  A synthetic primer

SEQ ID NO: 25 - DNA, 27 bp, Artificial Sequence
  A synthetic primer
aagaattctaagatggcagcggcggca

SEQ ID NO: 26 - DNA, 32 bp, Artificial Sequence
  A synthetic primer
aaaagcttctaactcccatagtgcatggtgtt

SEQ ID NO: 27 - DNA, 30 bp, Artificial Sequence
  A synthetic primer
aaggatccagccccagttatgagacatgat

SEQ ID NO: 28 - DNA, 30 bp, Artificial Sequence
  A synthetic primer
aaaagcttcttctttcactgagatgcatgc

SEQ ID NO: 29 - protein, 15 aa, Artificial Sequence
  A synthetic peptide
CDMRTVQVPDLETMR

SEQ ID NO: 30 - DNA, 21 bp, Artificial Sequence
  A synthetic primer
ctgaggtattaccagcagaca

SEQ ID NO: 31 - DNA, 19 bp, Artificial Sequence
  A synthetic primer
ctctctgatgcaggtctac

SEQ ID NO: 32 - DNA, 20 bp, Artificial Sequence
  A synthetic primer
ccggtggatgtggaatgtgt

SEQ ID NO: 33 - DNA, 21 bp, Artificial Sequence
  A synthetic primer
ctgaggtattaccagcagaca

SEQ ID NO: 34 - DNA, 19 bp, Artificial Sequence
  A synthetic primer
ctctctgatgcaggtctac

SEQ ID NO: 35 - DNA, 20 bp, Artificial Sequence
  A synthetic primer
ccggtggatgtggaatgtgt

SEQ ID NO: 36 - DNA, 30 bp, Artificial Sequence; identical to SEQ ID NO: 27
  A synthetic primer

SEQ ID NO: 37 - DNA, 30 bp, Artificial Sequence; identical to SEQ ID NO: 28
  A synthetic primer

SEQ ID NO: 38 - DNA, 31 bp, Artificial Sequence
  A synthetic primer
ggcgaattcactgcagcaggatggctcagtg

SEQ ID NO: 39 - DNA, 32 bp, Artificial Sequence
  A synthetic primer
gctgtcgacttgtggttggcctggcccccttg

SEQ ID NO: 40 - DNA, 45 bp, Artificial Sequence
  A synthetic primer
gagctggatcagggcgagctctgcacagaattcgtggggaccctg

SEQ ID NO: 41 - DNA, 45 bp, Artificial Sequence
  A synthetic primer
cagggtccccacgaattctgtgcagagctcgccctgatccagctc

SEQ ID NO: 42 - DNA, 24 bp, Mus musculus
ctgcttcagacctccatagatgtg

SEQ ID NO: 43 - DNA, 20 bp, Mus musculus
cagcgagaggtcgagtttgc

SEQ ID NO: 44 - DNA, 22 bp, Mus musculus
caacctgctggcttgcatccgg

SEQ ID NO: 45 - DNA, 25 bp, Mus musculus
tcagacctcttaatgaaggaaagca

SEQ ID NO: 46 - DNA, 24 bp, Mus musculus
gagaacaagtttcatttcccatca

SEQ ID NO: 47 - DNA, 28 bp, Mus musculus
ccagaacaaggcttgaagacccttccat